SEQUENCE LISTING (12 sequences)

SEQ ID NO: 1 - DNA, 21 bp, Artificial Sequence
  Synthetic
tagagggatgatagatggtga

SEQ ID NO: 2 - DNA, 19 bp, Artificial Sequence
  Synthetic
cttcatgagggacttgtac

SEQ ID NO: 3 - DNA, 19 bp, Artificial Sequence
  Synthetic
ggccatagcgctcactgat

SEQ ID NO: 4 - DNA, 21 bp, Artificial Sequence
  Synthetic
ccatagcctttgaagacccag

SEQ ID NO: 5 - DNA, 21 bp, Artificial Sequence
  Synthetic
ccccagcctttgaagacatag

SEQ ID NO: 6 - DNA, 32 bp, Artificial Sequence
  Synthetic
cctcccttgctggctgtgtcccaagcttaggc

SEQ ID NO: 7 - DNA, 31 bp, Artificial Sequence
  Synthetic
cgccccttcctttccgccatcctgcccccag

SEQ ID NO: 8 - DNA, 20 bp, Artificial Sequence
  Synthetic
gcgtggtattcagcaacggg

SEQ ID NO: 9 - DNA, 17 bp, Artificial Sequence
  Synthetic
tcgtgggtgtttccttc

SEQ ID NO: 10 - DNA, 1415 bp, Homo sapiens
gaattccgccctgcacccatgaccgcctcccaccagggccccgccctctgccccttttgggaaaccttctgcagatggatagaagaggcctactcaaatcctttctgaggttccgagagaaatatggggacgtcttcacggtacacctgggacccaggcccgtggtcatgctgtgtggagtagaggccatacgggaggcccttgtggacaaggctgaggccttctctggccggggaaaaatcgccatggtcgacccattcttccggggatatggtgtgatctttgccaatggaaaccgctggaaggtgcttcggcgattctctgtgaccactatgagggacttcgggatgggaaagcggagtgtggaggagcggattcaggaggaggctcagtgtctgatagaggagcttcggaaatccaagggggccctcatggaccccaccttcctcttccagtccattaccgccaacatcatctgctccatcgtctttggaaaacgattccactaccaagatcaagagttcctgaagatgctgaacttgttctaccagactttttcactcatcagctctgtattcggccagctgtttgagctcttctctggcttcttgaaatactttcctggggcacacaggcaagtttacaaaaacctgcaggaaatcaatgcttacattggccacagtgtggagaagcaccgtgaaaccctggaccccagcgcccccaaggacctcatcgacacctacctgctccacatggaaaaagagaaatccaacgcacacagtgaattcagccaccagaacctcaacctcaacacgctctcgctcttctttgctggcactgagaccaccagcaccactctccgctacggcttcctgctcatgctcaaataccctcatgttgcagagagagtctacagggagattgaacaggtgattggcccacatcgccctccagagcttcatgaccgagccaaaatgccatacacagaggcagtcatctatgagattcagagattttccgaccttctccccatgggtgtgccccacattgtcacccaacacaccagcttccgagggtacatcatccccaaggacacagaagtatttctcatcctgagcactgctctccatgacccacactactttgaaaaaccagacgccttcaatcctgaccactttctggatgccaatggggcactgaaaaagactgaagcttttatccccttctccttagggaagcggatttgtcttggtgaaggcatcgcccgagcggaattgttcctcttcttcaccaccatcctccagaacttctccatggccagccccgtggccccagaagacatcgatctgacaccccaggagtgtggtgtgggcaaaatacccccaacataccagatccgcttcctgccccgctga

SEQ ID NO: 11 - protein, 494 aa, Homo sapiens [sequence omitted]

SEQ ID NO: 12 - DNA, 7215 bp, Homo sapiens
aagttcccctgaaatatggctctggtcttcctccccttgccaatgaagaagatggcagtggaggttctatggcagccatcctggcctcactctgaggttccaatgaggattctgggcatcaagagacagctctgggcaaagctaaatcaagtcagcccctggacccagtgctgggctgctgggctttctgggagaacgccgctgggcttgctacacactcctcctcccagaaactccacacccacagccctgggtcttcctagccccgagactttcaagtccatatgcctggaatcccccttcctgagacccttaaccctgcatcctccacaacagaagacccctaaatgcacagccacactttgtcttaccctaataaaacccagacctttggattcctctcccctggaacccccagatccgcacaactttggggtgcattctcactctcagaccccaaatccaaagcccaagtgctcccctatgcaaatattccaaactcctcagttctacagcttatctgttgccccctcctaaatccacagccctgcggcacccctcctgaagtaccacagatttagtctggaggccccctctctgttcagctgccctggggtccccttatcctcccttgctggctgtgtcccaagctaggcaggattcatggtggggcatgtagttgggaggtgaaatgaggtaattatgtaatcagccaaagtccatccctctttttcaggcagtataaaggcaaaccaccccagccgtcaccatctatcatccctctaccaccatgctggcctcagggatgcttctggtggccttgctggcctgcctgactgtgatggtcttgatgtctgtttggcagcagaggaagagcaaggggaagctgcctccgggacccaccccattgcccttcattggaaactacctgcagctgaacacagagcagatgtacaactccctcatgaaggtgtcccaagacagggagatgggtgtctcggggtgggggctgcctagttggctggggctttgtggcagggggttgaccagtgtggaccagagtcttaggaaatggagttttggagtttcagcatcagaaagacaggatcttgggatgtccagctccctgactgtgagaacctgggtgcgaagcatcccagcacatgacatctcggtgctgggccccattcagagtggagggttctccctctaaccactcccacccacctccatcagatcagtgagcgctatggccccgtgttcaccattcacttggggccccggcgggtcgtggtgctgtgtggacatgatgccgtcagggaggctctggtggaccaggctgaggagttcagcgggcgaggcgagcaagccaccttcgactgggtcttcaaaggctatggtgcccaagagggggaaggtgggcaggtggacacgaaggtctcagtgttcccagccttctccctgactctcctgacaactggaggataagggagagtccccagtctggtcttccctccccatctccctacattggggcctctccatgtgtatccctcacctgtctccagcggccctgtcctgattcctccctgcctctctctgccccacctccttattctctctcactggagtctcctctttcccctctctctccatctctaaggacatcctgggtttctgtttaccagccctgggtctctgtctacatgagtctttgaggccctcttagcttctgggcttctctgggtttctcatctctccggatccctttctcaattcttcctctgtcttaggatgccagggttattcctacttccacatcttcaggctccatctcctggtaacagtctctcttccttccagaccctctctgtttctatctcaatattaaactctctgctccagctcagcttaagaatctcacaccaagagaggatgtcctccacccagatctccccatatctcactaccccaccctccatcctctgcctccatcactctctttctctccccactgccctgcggacgcgatccaatggagtgtggagctaatgccgtgaagctatgtgcatctctctgtctggccgtacctgggtaataacctgatcgactaggcgtggtattcagcaacggggagcgcgccaagcagctcctgcgctttgccatcgccaccctgagggacttcggggtgggcaagcgaggcatcgaggagcgcatccaggaggagtcgggcttcctcatcgaggccatccggagcacgcacggtgagcaggggaccccgagtgcgggggcaggagaaggaaaacacccaggacgaggaacccgcgcgcgttctgcctggggatggggactaggtggggaaaggcgcccgcacttccagccctggagtctggcgctgggaatttggctcaacaaggccctgcctcctggaattctgactctcctcagacctctgagttgactctctccccaacccccttctcccgacatacccggaggcgccaatatcgatcccaccttcttcctgagccgcacagtctccaatgtcatcagctccattgtctttggggaccgctttgactataaggacaaagagttcctgtcactgttgcgcatgatgctaggaatcttccagttcacgtcaacctccacggggcaggtaatggttgcagcccggcccgtgaaggcccttaccaaaaccggcaaattgttcccctaccgggggaagggggccccaaattcccaccgccccccggacagtgtcccctcaaaatcagtccccgatttgggcaaattggcagagtggaaccagacccgggttggttgtccaatcccctgctctccagggacaccgggatagcacaacagatgctccccaaaacagagcctgctggcaggatgcataccctcagctcagctctctcaccctgggcacgtgttcccatccccaacttaccggtaatttctaacagatgctccctacccaggtcttcttgaatattttaacacccggaaaccctgggtacctaaccttccctgtaaactttagagattagttcctatccggcccctctgaaatacctaaccaccggagaccagatgcctttaactcagttccttccttgctatgaaacaaatcccattcccatcagctcctgccccgtgacagctgtccttcccttcccatcctctctctgcaaccccagctctatgagatgttctcttcggtgatgaaacacctgccaggaccgcagcaacaggcctttcagttgctgcaagggctggaggacttcatagccaagaaggtggagcacaaccagcgcacgctggatcccaattccccacgggacttcattgactcctttctcatccgcatgcaggaggtacaccccagcagccactgcggggagatgcaaagccaggcagagggaaatcagtctgggagtggggcaggcagatgacacaggcccattcaaattaaccctcatcataataatcctcacaattggctgggtgccgtggctaacagcctgtaatcccagcactttgggaggccgaggcaggtggatcacctgaggtcaggagttcgagaccagcctggccaacatggtcaaaccccgtctctactaaaaatccaaaaattagttgggcatggtggcgcgaaggggggcagaggttgcaatgagccaagatcacggcattgcactccagtctgggtgacagaatgaggccctgtgtcaaaaaaaattaatcacttgtttaaaaagtaagtgagcctgcatggtcatgcgcatgtgcagctccagctactcaggaggctgaggctggaggattgcttgagctcaggagttggcgtccggcctgtgcaacttagcaagaccaagtcagtataagaaaaaaaaaaaacaaaaaaaaagctgacagctaagttgataattgacggacagatggtcagcaaggtaacgaaggtgagaaggaagagcattgggggcaacgccaggagtcagggcaagggctggttcctagagcgagtctggtaggatctagggcccctcttctccaccctgcggtcttgccccaaagagaggtcgagggtgctgggattgcgctagactcgagtctgtgtagatcttggggtcccctcttgacccccattggtctgaacctaagagtggaagatccatggggtgaacccctagatggtgccctgaggtcaagcaggagtgaggttgtcctaaagccccctctcccttcaggaggagaagaaccccaacacggagttctacttgaagaacctgatgatgagcacgttgaacctcttcattgcaggcaccgagacggtcagcaccaccctgcactatggcttcttactgctcatgaagcacccagaggtggagggtaaggctggagggggacggaagtggagggccccagaccctcaaaattccccttcgactggtgcaatgtccccacctgtcccagatcccgggaccctgagacgtgacttgctgtccagagacagggcaacattcagctggtaggcatcagctgagtctcattagatattaaaatattgaaaatgtctgcactgattggtcagtcacttctgtcccaagcccactgagtgcccactgcccgttccaccgggtcatcccctaagttcctccctgtgcctcccctgtgattctggcacaacctggttaacaggatcctactccaacaatgcgaatgggtgatgtctgttctgttatgaatgctctacttccgtctcataggcggaggcatttcatccaccccattttgcctatccggactatcatttcctgctctgagacccctagatacctaaacacattccccctcctcccccagccaaggtccatgaggagattgacagagtgatcggcaagaaccggcagcccaagtttgaggaccgggccaagatgccctacatggaggcagtgatccacgagatccaaagatttggagacgtgatccccatgagtttggcccgcagagtcaaaaaggacaccaagtttcgggatttcttcctccctaaggtgctatccgcccccaccccccagactacggggactccagcccctctctgtgtccccagcatcccacccacattagaagctttctagaccctgtcccactccctcaatcagtcaaaaaagacttccccaaccaccacatccgttccacctttccacttagacactcctgagtcctgcatctctccagactctttgtgtcaggagaatcaaacacatgttcccaaacttcctatcttaagaaacagaagccccctttccattcggccttttgtcatagggacagaaatctcaggtcccccaaactcctgcctagaaggacatggaccccatgtctcccaaacttcctgtttcagagatgtgaaccttctatcccccaaggtcctccctcagaggtccccaattcccatgcctgccacttcccctcaccggggcaccctagttccccctccagcccctgtgtactctcaacaatcccccaacccgcctcatcacatacaccttcctcctccctcccagggcatagaagtgttccctatgttgggctccgtgctgagagacctcaggttcttctccaacccccgggacttcaatccccagcacttcctgggtgagaaggggcagtttaagaagcgtgatgcttttgtgcccttctccatcagtaagagaccactgtttggtgccaggcttactactcacaccagcaggggcctcccttacccagttcccctctctgccgtgtagcctagtatttccccagcttggcaagttcctgttagcaatctaccgtcgagccaccaggtgatactcccttaactaccaagcacccagtacctgtgcccaggcaaaaggaaaggaaacatcatacccctttcagaggcgggggaaaaccaaaggccagagagaatcagagatttatttccctagggtcacacaggagattcttcagcatccctaaaaaggagatgacggcacagcaggtcatatttgggagttcttatctgggggaagggggatcttaaacctcccattgtggacacctggcatcgatcaaccccatcttttggtcatcttttgggtcactcaaggaaactgaggtcaaggagggtcaagaggctccctcttaaagtctctcagggccatatattccacccttcctccctgggagagccgcagctggaggtcggtactggggcgaggctgcactgagagtgggcttcacctccacccctcccgcctctcctcctcaggaaagcggaactgtttcggagaaggcctggccagaatggagctctttctcttcttcaccaccgtcatgcagaacttccgcctcaagtcctcccagtcacctaaggacattgacgtgtcccccaaacacgtgggctttgccacgatcccacgaaactacaccatgagcttcctgccccgctgagcgagggctgtgccggtgaaggtctggtgggcggggccagggaaagggcagggccaagaccgggcttgggagaggggcgcagctaagactgggggcaggatggcggaaaggaaggggcgtggtggctagagggaagagaagaaacagaagcggctcagttcaccttgataaggtgcttccgagctgggatgagaggaaggaaacccttacattatgctatgaagagtagtaataatagcagctcttatttcctgagcacgtacccccgtgtcacctttgttcaaaaaccattgcacgctcacctaatttgccacaaaacccccttcgaaggggcgttcatgcccattttacacgtgacaaaactgaggcttagaaagttgtctctgatgtctcacaaaacataagtgcccagaaaatctgcgaacacagatctgtgcccatagccttctagacagattcttaaaaagcacctattcctcacgcaaaacagtttagtatagaatcacatggcctgaacatccctgtccgggggagttccccagagacctggggggtggttgccctgccttcactgcacacatgcccacactctcacctactcaacatgctgtgactacccgggtgtaatctgtgcttgctaccagataaggccactgtagcccattcagagtcagcccagggacacaacgagacatgactggacatacagggtcagtccattaacaa

(19) United States
(12) Patent Application Publication
(10) Pub. No.: US 2009/0285797 A1
(43) Pub. Date: Nov. 19, 2009

(54) USE OF INHIBITORS OF CYP2A6 FOR REGULATING NICOTINE METABOLISM

(75) Inventors: Edward M. Sellers, Toronto (CA); Rachel F. Tyndale, Toronto (CA)

Correspondence Address: BERESKIN AND PARR LLP/S.E.N.C.R.L., s.r.l., 40 KING STREET WEST, BOX 401, TORONTO, ON, M5H 3Y2, CA

(73) Assignee: Nicogen Inc., St. Laurent (CA)

(21) Appl. No.: 12/237,713

(22) Filed: Sep. 25, 2008

Related U.S. Application Data
(63) Continuation of application No. 10/715,548, filed on Nov. 19, 2003, now abandoned.
Application No. 10/715,548 is a division of application No. 09/214,851, filed on Sep. 9, 1999, now abandoned.
(60) Provisional application No. 60/021,940, filed on Jul. 17, 1996.

(51) Int. Cl. A61K 31/366; A61K 39/395; A61K 31/7048; A61K 31/4174; A61K 31/4164

(52) U.S. Cl. 424/130.1; 514/457; 514/455; 514/456; 514/27; 514/342; 514/399; 514/396; 514/470

(57) ABSTRACT

The invention relates to methods and compositions for regulating nicotine metabolism. Also provided are methods for screening and assessing substances for regulating nicotine metabolism. Methods are provided for assessing nicotine metabolism.

### FIELD OF THE INVENTION

The invention relates to methods for regulating nicotine metabolism in an individual; compositions for regulating nicotine metabolism in an individual; methods of treating conditions requiring regulation of nicotine metabolism in an individual; methods of screening for a substance that regulates nicotine metabolism in an individual; and methods of assessing nicotine metabolism in individuals.

### BACKGROUND ART

Nicotine is the primary alkaloid present in tobacco playing a crucial role in establishing and maintaining tobacco dependence. Several studies have shown that smokers adjust their smoking behaviour to try and maintain constant nicotine blood levels, and hence brain nicotine levels. Studies using different nicotine yield cigarettes (Finnegan et al., 1945), nicotine replacement therapy [Lucchesi et al. (intravenous infusion), 1967; Jarvik et al., 1970 (ingestion); Kaslowski et al., 1975; Russell et al., 1976; Ebert et al., 1984 (nicotine chewing gum); Levin et al., 1994 (nicotine patches)], nicotine blockade (Stolerman et al., 1973, Nemeth-Coslett et al., 1986; Rose et al., 1994), and alteration of urinary pH (Benowitz et al., 1983; 1985; Rosenburg et al., 1980), showed that nicotine intake can be regulated to avoid exceeding the blood nicotine concentration of typical smoking levels. The studies provide clear evidence that smoking behaviour is modified in smokers to regulate nicotine blood levels. Therefore, changes in nicotine clearance from the body, such as metabolic changes, can have a significant impact on smoking behaviour.

Nicotine and its metabolites have been extensively studied over the past few decades. Nicotine is, for the most part, metabolized in the liver (80%), and to a smaller extent in the lungs and kidneys (Schielvelbein, 1982; Turner, 1975). The major metabolite of nicotine is cotinine (Benowitz et al., 1994). Nicotine is primarily metabolized to cotinine through a two step process (FIG. 1). The first step in the process produces the intermediate, nicotine-Δ-^{1′(5′)} iminium ion (Peterson and Castagnoli, 1988, Williams et al., 1990), which is then further oxidized through a cytosolic aldehyde oxidase reaction in the presence of liver microsomes, O₂ and NADPH (Hill et al., 1972; Peterson et al., 1987; Brandage et al., 1979; Gorrod et al, 1982).

The cytochrome P450 (CYP) system has been implicated in the metabolism of nicotine. Evidence for CYP involvement in nicotine metabolism has come from rat liver studies in which reconstituted purified CYPs, and specific antibodies were shown to inhibit nicotine metabolism. In particular, rat studies have shown that phenobarbital inducible CYPs (i.e., the CYPs; −2B1, −2B2, −2C6, and −3A2) are involved in nicotine metabolism (Nakayama et al., 1982; Hibberd and Gorrod 1985; Foth et al., 1990; Seaton et al., 1991 and 1993). Of 12 human CYPs forms tested, CYP2B6 showed the highest nicotine oxidase activity while CYP2E1 and CYP2C9 showed intermediate levels (Flammang et al., 1992). McCracken et al., (1992), have shown that human CYP2B6 and CYP2D6 displayed high rates of nicotine to cotinine metabolism, whereas the catalytic activity of CYP2E1 towards nicotine is not detectable. The results concerning CYP2E1 and CYP2D6 are in disagreement with the findings of Flammang et al., (1992). Thus, there remains some ambiguity concerning the affinities of CYPs for nicotine.

The CYP2B proteins are expressed at low amounts in the liver (less than 5% of the total hepatic CYP content) in all animals and humans, but their levels can be highly induced by exposure to a number of diverse chemicals, including the prototypic CYP2B inducer phenobarbital (Ryan et al., 1990; Guengerich et al., 1982b). The human CYP2B6 enzyme is expressed at variable levels among different individuals. CYP2B6 has poor oxidation activity towards benzopyrene, 7-ethoxycoumarin, coumarin, ethoxyresorufin, pentoxyresorufin, ethylmorphine, benphetamine, and aniline (Mimura et al., 1993). Orphenadrine, an anti-parkinsonian agent, was found to be a specific inhibitor of CYP2B6 (Reidy et al., 1992; Chang et al., 1993).

cDNA studies have implicated CYP2B6, CYP2C9, CYP2D6 and CYP2E1 and have provided a possible role for CYP2A6 in nicotine metabolism in isolated expression systems (Flammang et al., 1992; McCracken et al., 1992). CYP2A6 also displays a genetic polymorphism whereby certain individuals contain an inactive enzyme (Daly et al., 1994; Fernandez-Salguero et al., 1995). CYP2A6 is the predominant, if not the only, coumarin 7-hydroxylase in humans (Pearce et al., 1992). CYP2A6 catalyzes the hydroxylation of coumarin a naturally occurring compound in plants and essential oils (Pelkonan et al., 1985; Raunio et al., 1988; Yamano et al., 1990; Pearce et al., 1992). In primates, such as humans and baboons, coumarin is metabolized to 7-hydroxycoumarin (˜80%) (Cholerton et al., 1992; Shilling et al., 1969; Moran et al., 1987). However, in rodent species such as the rat, mouse and hamster, 3-hydroxycoumarin is the major metabolite (Shilling et al., 1969; Egan et al., 1990). Early experiments on coumarin 7-hydroxylase activity, in human liver microsomes, demonstrated marked inter-individual differences in the expression levels of CYP2A6 (Kapitulnik et al., 1977; Pelkonen et al., 1985). Variability was also found in levels of expression of CYP2A6 mRNA in human livers (Miles et al., 1990; Yamano et al., 1990; Yun et al., 1991). In particular, CYP2A6 protein levels in human liver microsomes varied by over 100 fold (Yun et al., 1991). CYP2A6 also has been found to metabolize several procarcinogens such as NNK (Crespi et al., 1991), aflaxtoxin B1 (Yun et al., 1991); hexamethylphosphoramide (Ding et al., 1988), and nitrosodimethylamine (Davies et al., 1989; Fernandez et al., 1995).

### DISCLOSURE OF THE INVENTION

The present inventors have found that variation in nicotine metabolism among individuals is due to variable expression of CYP2A6, and not CYP2D6. CYP2A6 has been shown to be the major nicotine metabolizing enzyme in human livers. Coumarin, a specific CYP2A6 substrate, was found to specifically and selectively inhibit nicotine metabolism to cotinine by 84%±11% in test livers, and addition of orphenadrine (a CYP2B6 inhibitor) enhanced the inhibition. Methoxsalen (a.k.a. 9-Methoxy-7H-furol[3,2-g][1]benzopyran-7-one; 6-hydroxy-7-methoxy-5-benzofuranacrylic acid δ-lactone; 9-methoxypsoralen; 8-methoxy-4′,5′:6,7-furo-coumarin; 8-methoxy[furano-3′,2′:6,7-coumarin]; ammoidin; xanthotoxin; 8-methoxypsoralen; 8-MOP; 8-MP; Meladinine; Meloxine; Oxsoralen) has also been found to be a potent inhibitor of CYP2A6 and thus of nicotine to cotinine metabolism. A monoclonal antibody raised against CYP2A6 also inhibited cotinine formation; however, antibodies to other CYPs did not significantly inhibit cotinine formation. The amount of CYP2A6, as determined by Western blots, was highly correlated to Vₘₐₓ (r=0.83, p<0.001), and to inhibition by coumarin (r=0.80, p<0.001). The data indicate that variability in CYP2A6 expression results in inter-individual variation in nicotine metabolism, which in turn, can have behavioural consequences such as smoking more or less cigarettes. Therefore, selective and specific inhibitors of CYP2A6 can be used to regulate nicotine metabolism, and in particular substantially decrease nicotine metabolism, thereby affecting tobacco use.

As used throughout this specification, the terms “inhibitor” and “inhibition”, in the context of the present invention, are intended to have a broad meaning and encompass substances which directly or indirectly (e.g., via reactive intermediates, metabolites and the like) act on CYP2A6 to inhibit or otherwise regulate the ability of CYP2A6 to catalyze metabolism of nicotine. Other substances which act indirectly on CYP2A6 include those substances which inhibit transcription and/or translation of the gene encoding CYP2A6.

Broadly stated, in one of its aspects, the present invention relates to a method of regulating nicotine metabolism in an individual comprising selectively inhibiting CYP2A6. Inhibition of CYP2A6 may be achieved using one or more of the following: (i) substances which inhibit CYP2A6 activity; or (ii) substances which inhibit transcription and/or translation of the gene encoding CYP2A6. CYP2A6 may also be selectively inhibited by interfering with the transcription or translation of the gene encoding CYP2A6 using gene transfer methods.

The present invention also provides a method of screening for a substance that regulates nicotine metabolism to cotinine in an individual comprising assaying for a substance which selectively (i) inhibits CYP2A6 activity; or (ii) inhibits transcription and/or translation of the gene encoding CYP2A6.

The invention further provides a pharmaceutical composition for use in treating a condition requiring regulation of nicotine metabolism to cotinine comprising an effective amount of a substance which selectively inhibits CYP2A6, and/or a pharmaceutically acceptable carrier, diluent, or excipient. A method is also provided for treating a condition requiring regulation of nicotine metabolism to cotinine in an individual comprising administering to the individual an effective amount of a substance which selectively inhibits CYP2A6.

The present invention also provides the use of a substance which selectively inhibits CYP2A6 for the preparation of a medicant for regulation of nicotine metabolism to cotinine in an individual.

CYP2B6 inhibitors may also be used in combination with inhibitors of CYP2A6 to provide an enhanced inhibitory effect. Therefore, the present invention provides a method for enhancing inhibition of nicotine metabolism by a CYP2A6 inhibitor in an individual comprising administering to the individual an effective amount of a substance which selectively inhibits CYP2A6, and an effective amount of an inhibitor of CYP2B6. A pharmaceutical composition for use in treating a condition requiring regulation of nicotine metabolism to cotinine is also provided comprising an effective amount of a substance which selectively inhibits CYP2A6, an effective amount of an inhibitor of CYP2B6, and/or a pharmaceutically acceptable carrier, diluent, or excipient. Further, a method for treating a condition requiring regulation of nicotine metabolism to cotinine in an individual is provided comprising administering to the individual an effective amount of a substance which selectively inhibits CYP2A6, and an effective amount of an inhibitor of CYP2B6.

The pharmaceutical compositions and methods may be used to diminish a subjects desire for nicotine and thereby can be used to alter tobacco use.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood with reference to the drawings in which:

FIG. 1 shows the two step conversion of nicotine to cotinine;

FIG. 2A shows the amino acid and nucleotide sequence for cytochrome CYP2A6;

FIG. 2B shows the mRNA sequence for cytochrome CYP2B6;

FIG. 3 shows the protein-time curves of cotinine production from 100 μM nicotine in the presence of 20 μl rat cytosol by K20 human microsomes;

FIG. 4 shows sample Michaelis-Menten curves, with inset Eddie-Hofstee plots, of nicotine to cotinine metabolism where (A) is a graph displaying single enzyme kinetics for the L29 human liver and (13) displays multiple enzyme site kinetics for the L30 human liver;

FIG. 5 is a bar graph showing apparent Kₘ values of nicotine to cotinine metabolism by 30 human liver microsomes;

FIG. 6 is a bar graph showing apparent Vₘₐₓ values of nicotine to cotinine metabolism by 30 human liver microsomes;

FIG. 7 shows antibody activity (data from Gentest Corp.); where (A) is a graph of inhibition of coumarin oxidation by MAB-2A6 antibody and (B) is a graph of inhibition of testosterone 16β-hydroxylation (CYP2B1), and lidocaine methyl-hydroxylation (CYP2B2) by anti-CYP2B1 antibody;

FIG. 8 shows Western blots of increasing concentrations of L64 microsomal protein (above) with respective densities plotted to show linearity of analysis (below);

FIG. 9 is a bar graph showing coumarin (150 μM) inhibition of cotinine formation by 30 human liver microsomes;

FIG. 10 is a bar graph showing percent inhibition of nicotine (100 μM) to cotinine metabolism by 150 μM coumarin by 30 human liver microsomes;

FIG. 11 is a Dixon plot of coumarin inhibition of cotinine formation in K27 liver microsomes;

FIG. 12 is a bar graph showing the use of antisense oligodeoxynucleotides (ASO) for the reduction of the CYP2A6 enzyme.

FIG. 13 is a graph showing the effects of MAB-2A6 on nicotine (100 μM) to cotinine metabolism by K27 human liver microsomes;

FIG. 14 shows Western blots of 30 human liver microsomes; each blot is accompanied by a 15, 30, 75 and 100 μg lanes of L64 microsomal protein such that individual blots can be compared;

FIG. 15 is a graph showing the correlation between nicotine to cotinine Vₘₐₓ values and the amount of immunoreactive CYP2A6 by 30 human livers;

FIG. 16 is a graph showing the correlation between CYP2A6 mediated cotinine formation and the amount of immunoreactive CYP2A6 by 30 human livers;

FIG. 17 is a graph showing the correlation between nicotine to cotinine Vₘₐₓ/Kₘ values and the amount of immunoreactive CYP2A6 by 30 human livers;

FIG. 18 is a bar graph showing orphenadrine (150 μM) inhibition of cotinine formation by 30 human liver microsomes;

FIG. 19 is a bar graph showing the percent inhibition of cotinine formation by orphenadrine (150 μM) using 30 human liver microsomes;

FIG. 20 is a graph showing the effects of anti-rat CYP2B1 on cotinine formation by K27 human liver microsomes;

FIG. 21 is a bar graph showing coumarin and orphenadrine (150 μM each) inhibition on nicotine metabolism by 30 human liver microsomes; and

FIG. 22 is a bar graph showing troleandomycin (150 μM) inhibition of cotinine formation by 30 human liver microsomes;

FIG. 23 shows chemical structures of some representative CYP2A6 inhibitors;

FIG. 24 is a table showing statistical results from clinical studies using methoxsalen to inhibit the activity of CYP2A6 on nicotine to cotinine metabolism;

FIG. 25 is a graph showing the effects over time of methoxsalen on the nicotine plasma concentrations of seven subjects;

FIG. 26 is a graph showing the effects over time of methoxsalen on the cotinine plasma concentrations of seven subjects;

FIG. 27 is a bar graph summarizing significant subjective effect changes associated with methoxsalen increased plasma nicotine concentrations in seven subjects;

FIG. 28A is a graph showing a subjective rating of current nausea in seven subjects involved in the clinical study of the effect of methoxsalen on plasma nicotine concentrations;

FIG. 28B is a graph showing a subjective rating of current desire for a cigarette in seven subjects involved in the clinical study of the effect of methoxsalen on plasma nicotine concentrations;

FIG. 28C is a graph showing a subjective rating of current pleasantness of a cigarette in seven subjects involved in the clinical study of the effect of methoxsalen on plasma nicotine concentrations;

FIG. 29 is a graph showing the inhibitory effect on cotinine formation from nicotine of various antibodies including the CYP2A6 antibody;

FIG. 30A is a table showing the inhibition of the nicotine to cotinine metabolism by various chemical compounds;

FIG. 30B is a table of Kᵢ values for the inhibition of the CYP2A6 substrate coumarin to 7-hydroxycoumarin metabolism by various compounds;

FIG. 30C is a table showing the percent inhibition of various compounds on cotinine formation from nicotine;

FIG. 30D is a bar graph showing the percent inhibition of the nicotine to cotinine metabolism by various chemical compounds;

FIG. 31 is a Dixon plot of 7-methoxycoumarin inhibition of nicotine to cotinine formation in K28 human liver microsomes;

FIG. 32 is a Dixon plot of methoxsalen inhibition of nicotine to cotinine formation with 10 minute preincubation in K28 human liver microsomes;

FIG. 33 is a Cornish-Bowden plot of methoxsalen inhibition of nicotine to cotinine formation with 10 minute preincubation in K28 human liver microsomes;

FIG. 34 is a graph showing the effect of pre-incubation time of methoxsalen (100 nM) on the inhibition of nicotine (30 μM) to cotinine formation in K26 human liver microsomes;

FIG. 35 is a Dixon plot of naringenin inhibition of nicotine formation with 10 minute preincubation in K26 human liver microsomes; and

FIG. 36 is a Dixon plot of diethyldithiocarbamic acid inhibition of nicotine to cotinine formation with 10 minute preincubation in K26 human liver microsomes.

FIG. 37 is a graph illustrating a correlation between fasted morning and non-fasted afternoon coumarin (C) testing sessions.

FIG. 38 is a graph showing the metabolism of nicotine over one hour in seven subjects.

FIG. 39 is a graph showing a time course of total 7-hydroxycoumarin concentration detected in the plasma of subjects given 100 mg of coumarin.

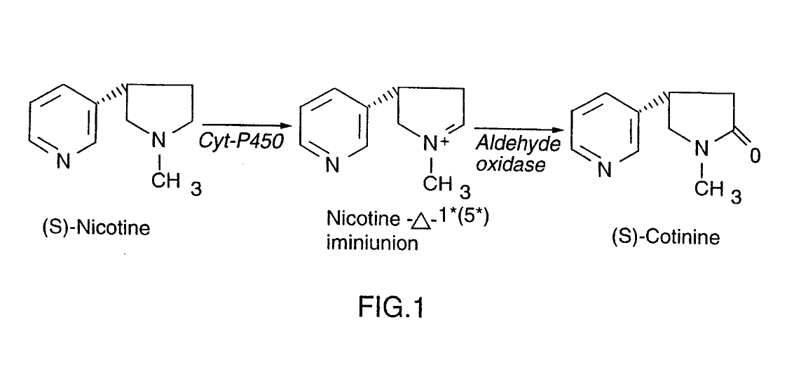

### BEST MODE FOR CARRYING OUT THE INVENTION

### 1. Method of Regulating Nicotine Metabolism in a Subject

As hereinbefore mentioned, in one of its aspects, the present invention relates to a method of regulating nicotine metabolism to cotinine in an individual comprising selectively inhibiting CYP2A6. Inhibition of CYP2A6 may be achieved using one or more of the following (i) substances which inhibit CYP2A6 activity; or (ii) substances which inhibit transcription and/or translation of the gene encoding CYP2A6.

Substances which inhibit CYP2A6 activity include substances which specifically bind to CYP2A6 and thereby inhibit its activity. Examples of such substances include antibodies which are specific for CYP2A6 including for example, the monoclonal antibody described by Pearce, R., et al, 1992., and commercially available antibodies such as MAB2A6 and monoclonal CYP2A6, sold by Gentest Corporation, Woburn, Mass., U.S.A.; XenoTech 2A6 sold by XenoTech LLC, Kansas City, Kans., U.S.A and polyclonal CYP2A6 sold by Research Diagnostics, Inc, Flanders, N.J., U.S.A.

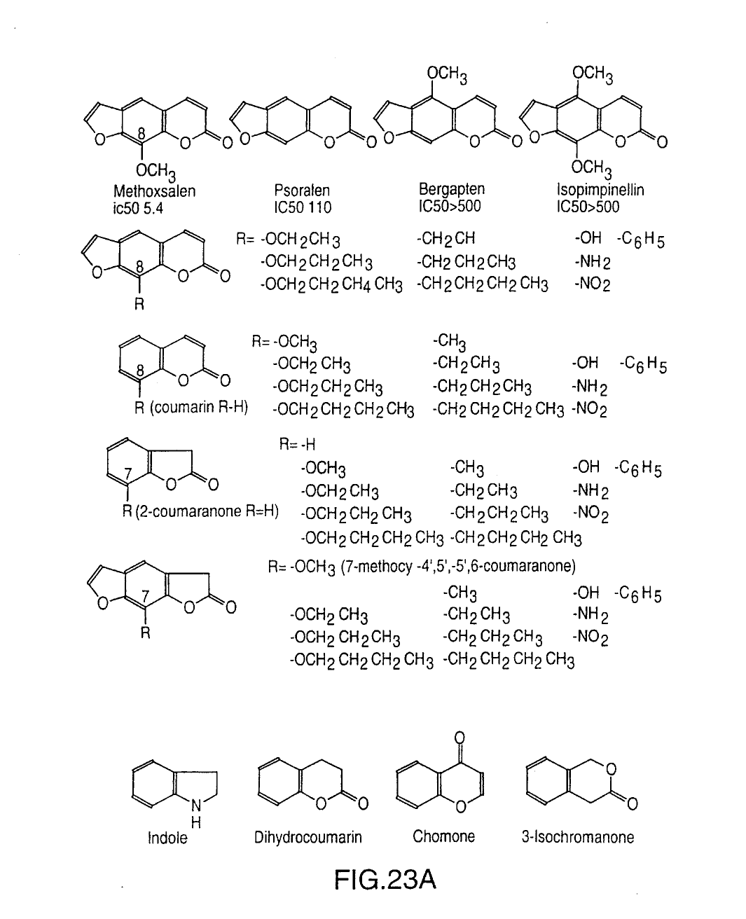

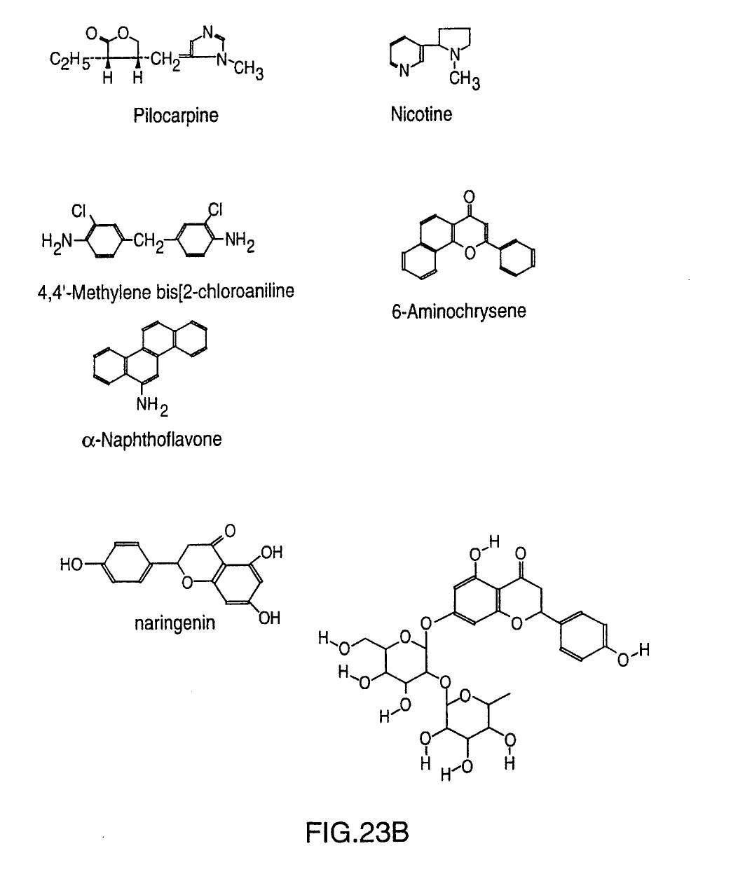

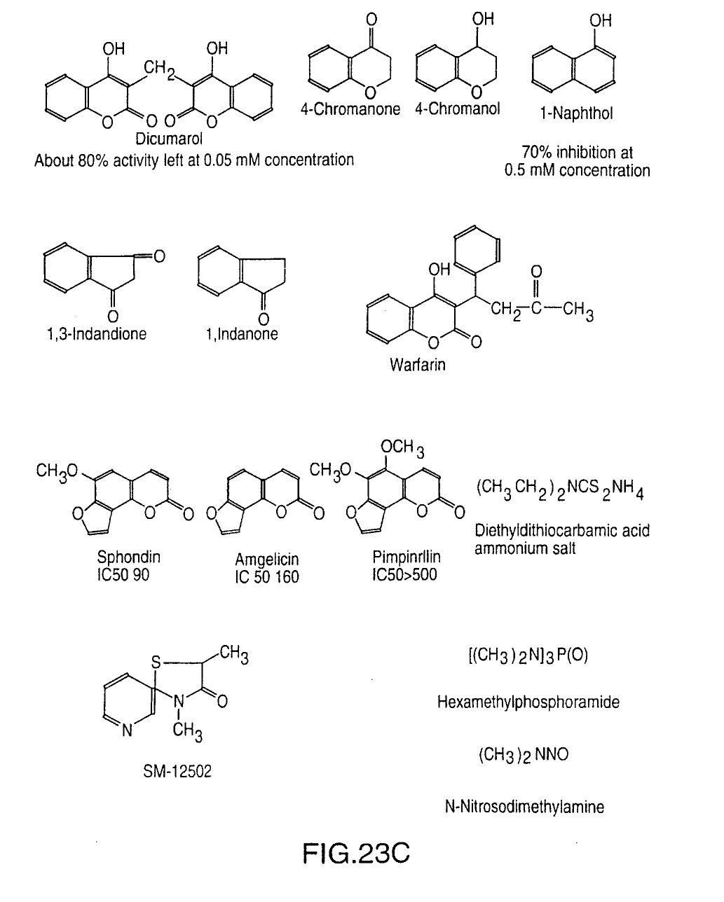

Substances which inhibit CYP2A6 activity also include substances having a lactone structure with a carbonyl oxygen. Non-limiting examples of such substances include coumarin (The Merck Index, Eleventh Edition Budavari, S., ed. Merck & Co. Inc., 1989, No. 2563), furanocoumarin, methoxsalen (The Merck Index, No. 5911), imperatorin (The Merck Index, No. 4839), psoralen (The Merck Index, No. 7944), α-naphthoflavone, isopimpinellin, (β-naphthoflavone, bergapten (The Merck Index, No. 1173), sphondin, coumatetralyl (racumin), and (+)-cis-3,5-dimethyl-2-(3-pyridyl)-thiazolidim-4-one (SM-12502) (Nunoya, et al., JPET 277:768-774, 1996). Other substances which inhibit CYP2A6 and can be used in the methods and compositions of the invention include naringenin and related flavones, diethyldithiocarbamate, nicotine (useful primarily in the screening methods of the invention), N-nitrosodialkylamine (e.g. N-nitrosodiethylamine (The Merck Index, No. 6557), N-nitrosodimethylamine (The Merck Index, No. 6558)), nitropyrene, menadione (The Merck Index, No. 5714), imidazole antimycotics, miconazole (The Merck Index, No. 6101), clotrimazole (The Merck Index, No. 2412), pilocarpine (The Merck Index, No. 7395), hexamethylphosphoramide, 4-methylnitrosamine-3-pyridyl-1-butanol, aflatoxin B (The Merck Index, No. 168). See FIGS. 23A to 23C for the chemical structures of these and other non-limiting representative inhibitors.

Derivatives and analogs of these substances may also be used in the methods and compositions of the invention. By way of example, derivatives of coumarin and methoxsalen include pharmaceutically acceptable salts, esters and complexes of coumarin and methoxsalen including potassium and sodium salts, and amino acid, carbohydrate and fatty acid complexes. In one embodiment, suitable analogs of coumarin may be selected based upon their functional similarity to coumarin, including the ability to inhibit the metabolism of nicotine to cotinine by CYP2A6. Examples of functional analogs of coumarin include 7-methoxycoumarin, 7-methylcoumarin, and 7-ethoxycoumarin and all structures shown in FIGS. 23A, 23B, 23C. Analogs of coumarin may also be selected based upon their three dimensional structural similarity to coumarin—i.e., the lactone/carbonyl structure.

The above lists of substances which inhibit CYP2A6 are provided by way of example only and should not be seen as limiting the scope of this invention. Additional substances which inhibit CYP2A6 activity may be identified using the screening methods described herein.

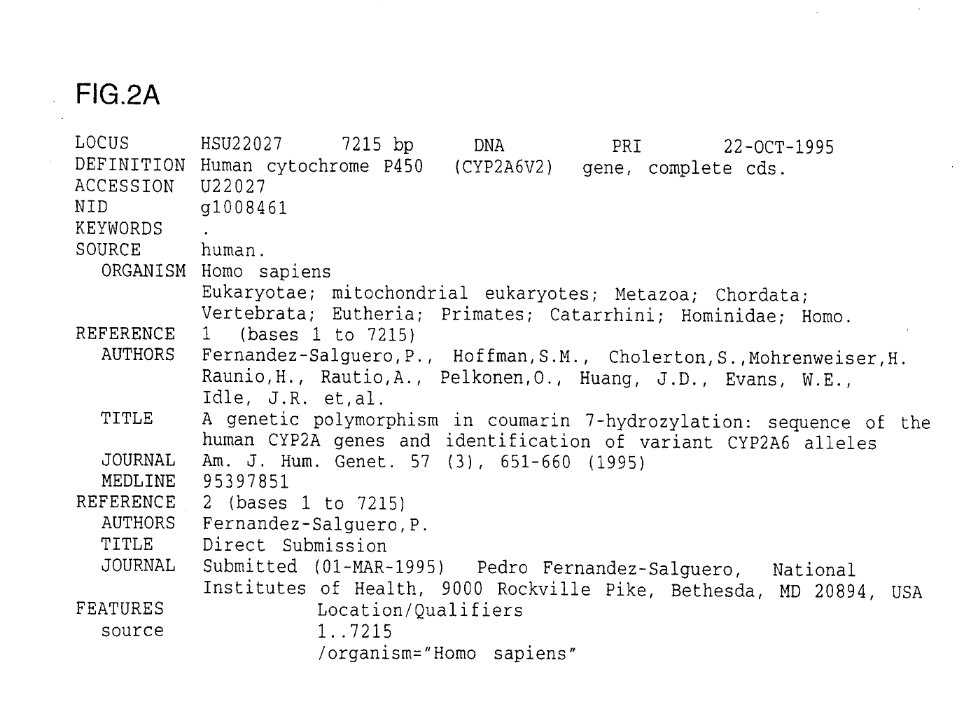

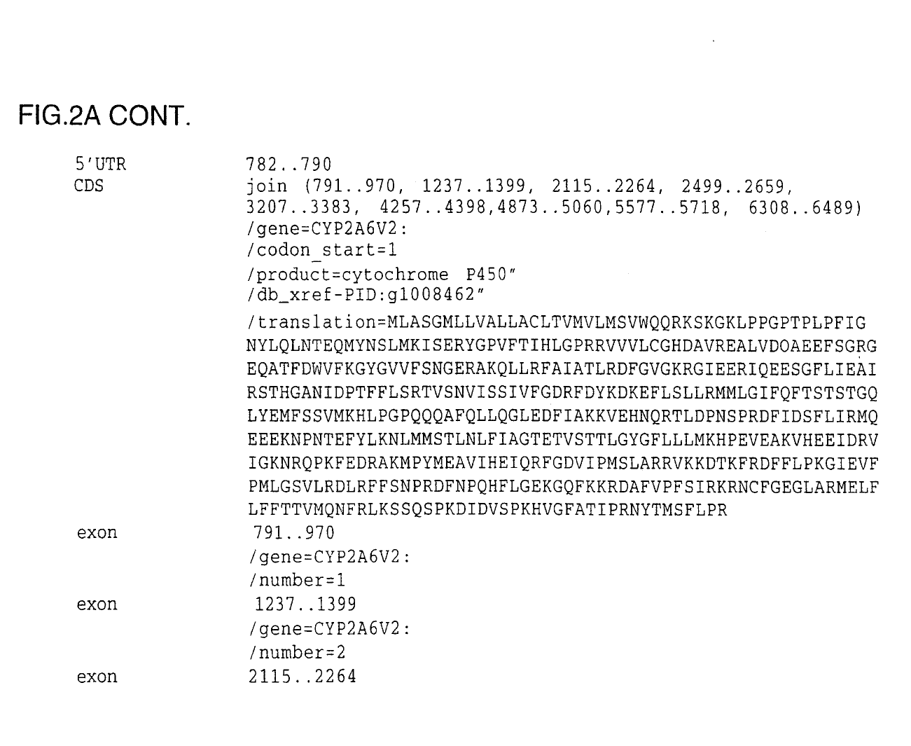

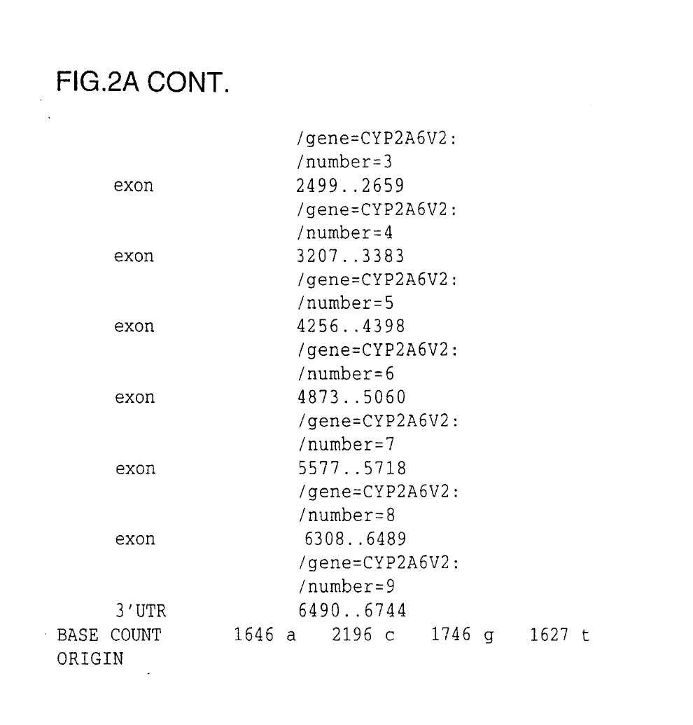

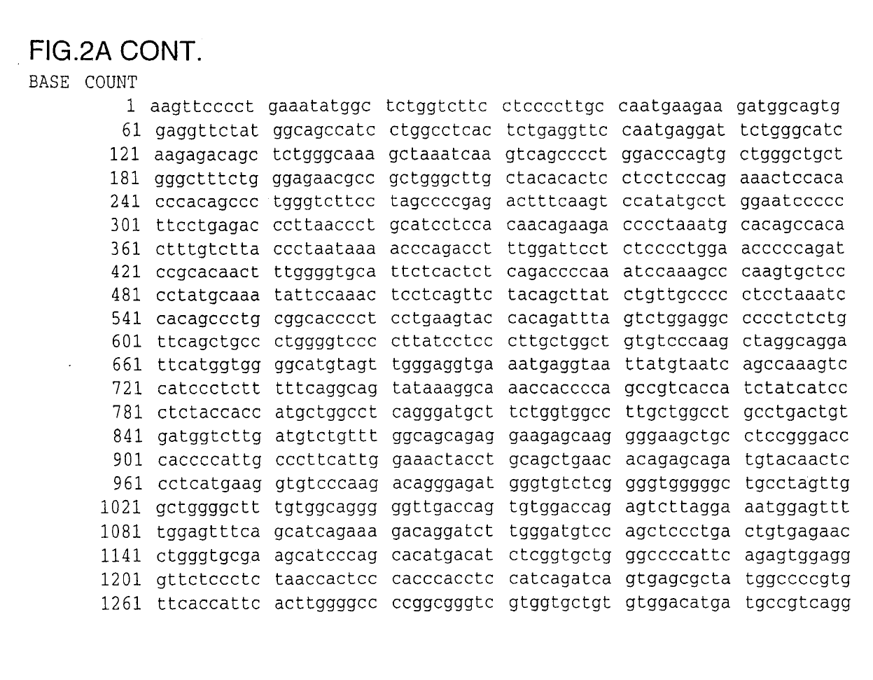

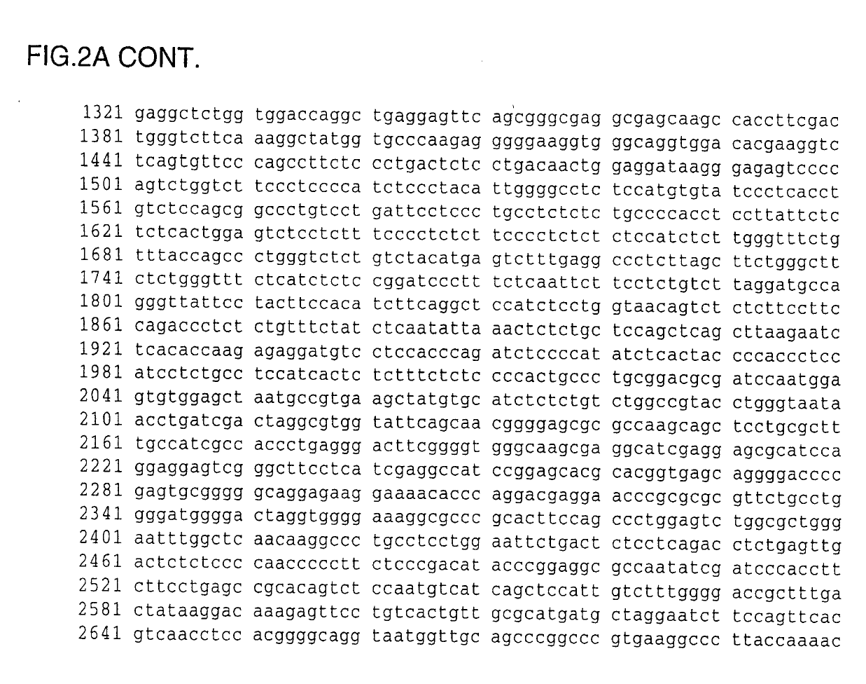

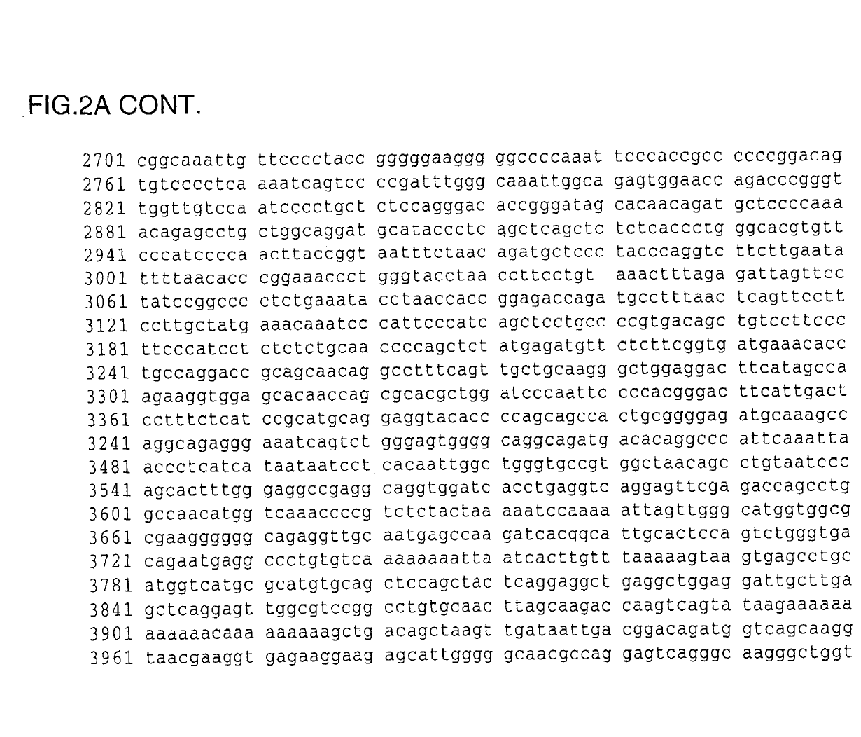

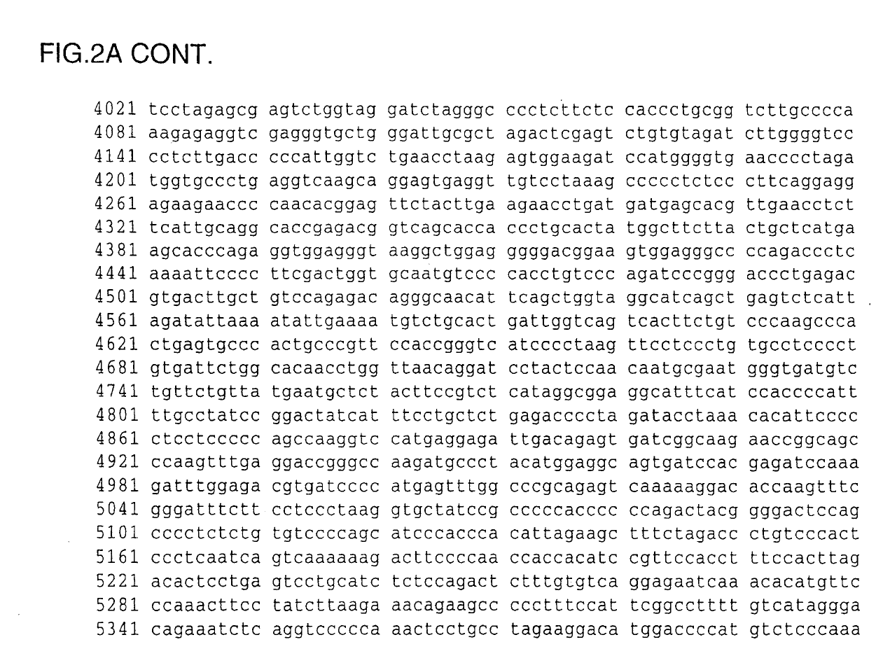

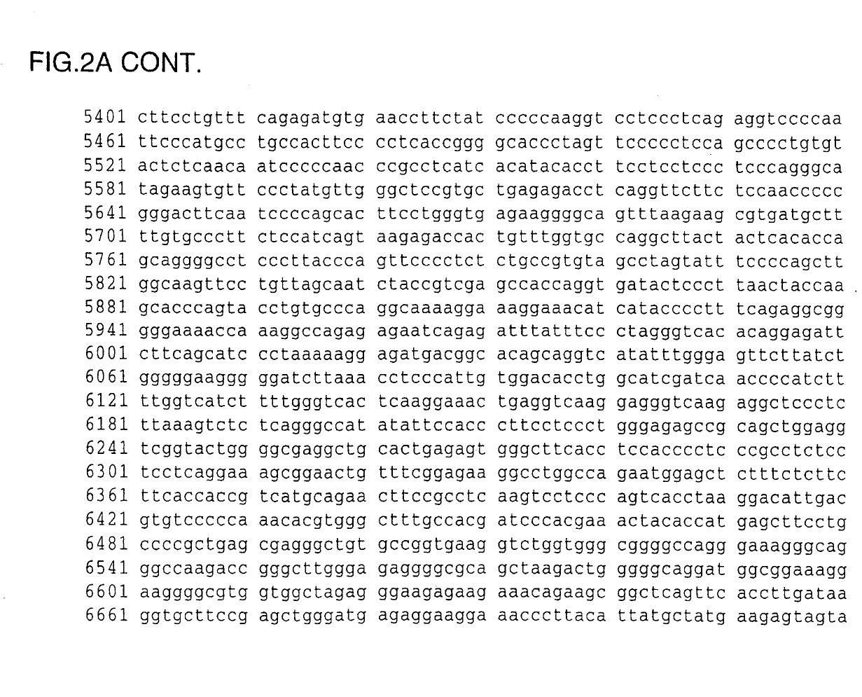

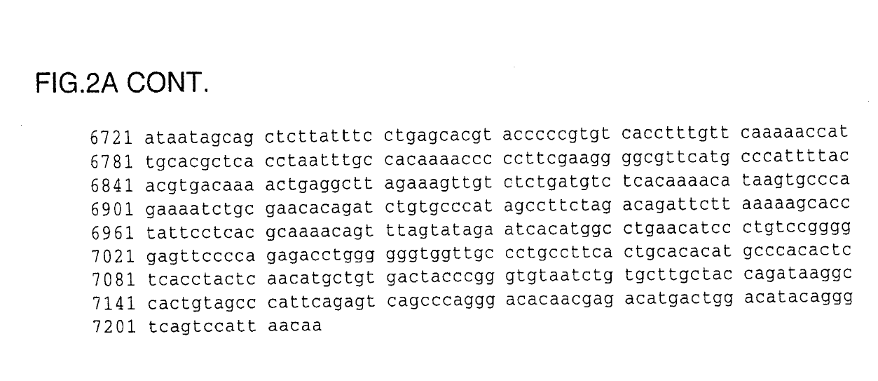

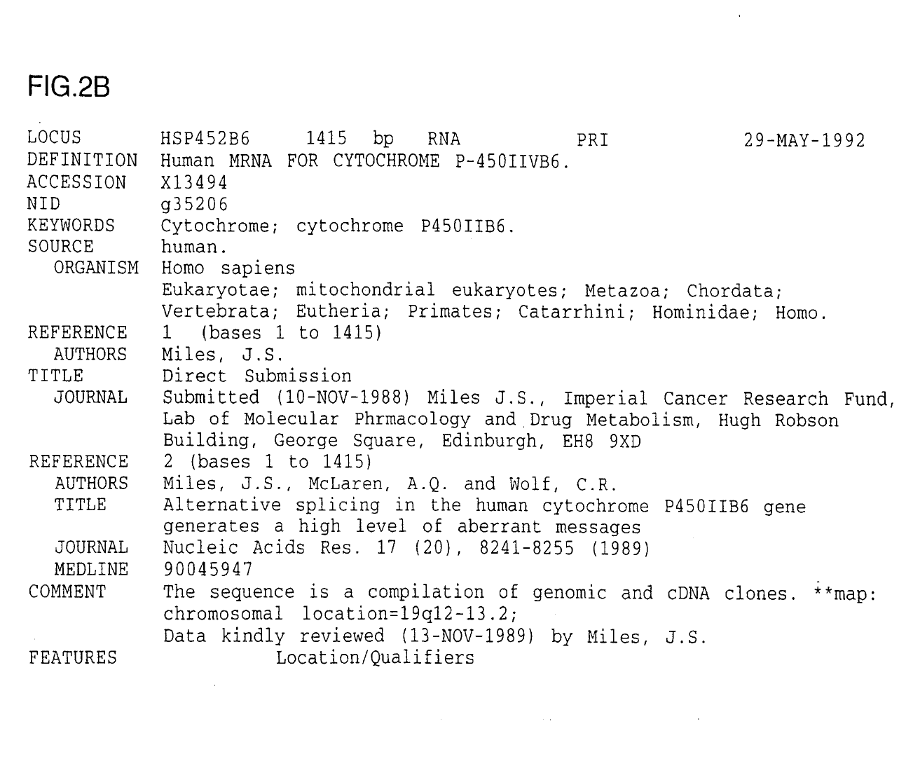

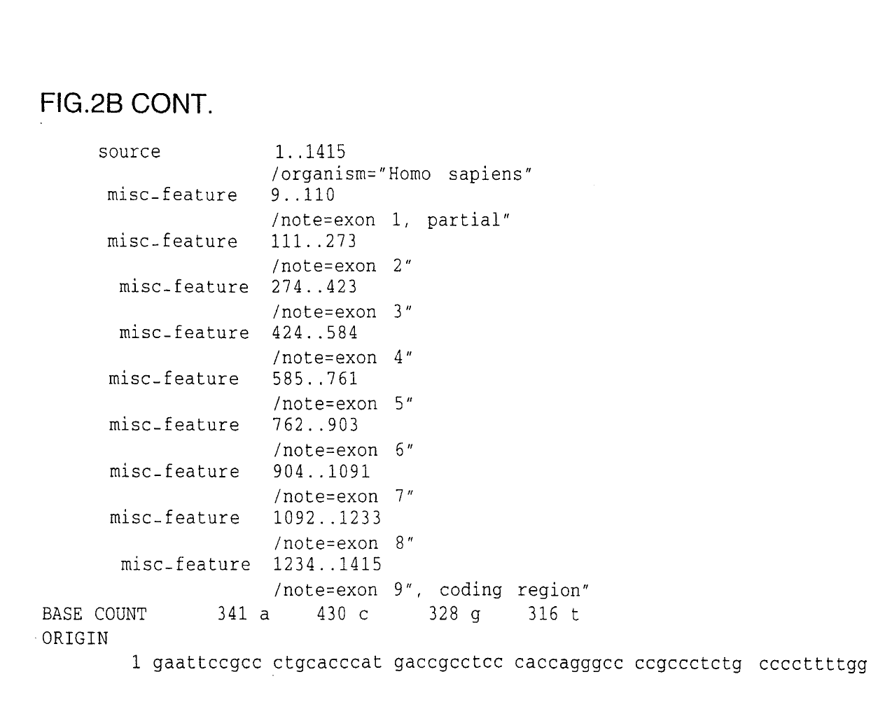

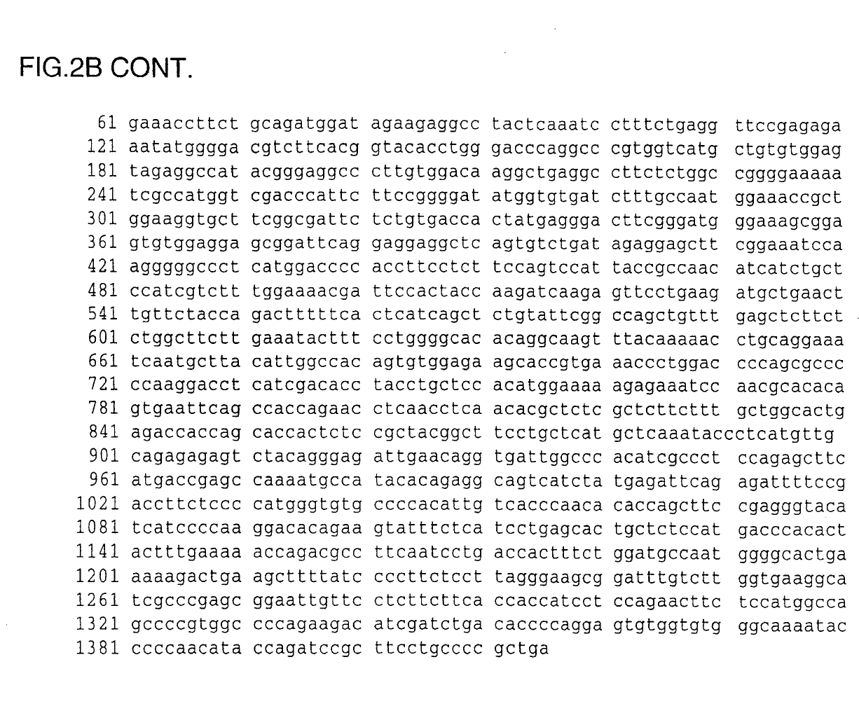

Substances which inhibit transcription and/or translation of the gene encoding CYP2A6 include a nucleic acid sequence encoding the CYP2A6 gene (see FIG. 2A, GenBank Accession No. HSU22027) or parts thereof (e.g., the region which is about 20 nucleotides on either side of nucleotide 790 (ATG), and the splice sites 1237, 2115, 2499, 3207, 4257, 4873, 5577 and 6308), inverted relative to their normal orientation for transcription—i.e., antisense CYP2A6 nucleic acid molecules. Such antisense nucleic acid molecules may be chemically synthesized using naturally occurring nucleotides or variously modified nucleotides designed to increase the biological stability of the molecules or to increase the physical stability of the duplex formed with CYP2A6 mRNA or the CYP2A6 gene. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

A nucleic acid molecule containing the antisense sequences may be introduced into cells in a subject using conventional techniques, such as transformation, transfection, infection, and physical techniques such as electroporation or microinjection. Chemical methods such as coprecipitation and incorporation of DNA into liposomes may also be used to deliver antisense sequences. The molecules may also be delivered in the form of an aerosol or by lavage. Suitable vectors or cloning vehicles for transferring the nucleic acid molecules are known in the art. Examples of suitable vectors include retroviral vectors, adenoviral vectors, and DNA virus vectors.

The ability of a substance to selectively inhibit CYP2A6 and thus regulate nicotine metabolism to cotinine may be confirmed using the methods described herein for screening for an inhibitor.

In one embodiment of the invention, the CYP2A6 inhibitor is at least one member selected from the group comprising coumarin, methoxsalen, derivatives thereof and analogs thereof (see FIG. 23A). Initial in vitro screening and clinical studies have identified that methoxsalen is a potent inhibitor of CYP2A6.

CYP2A6 may also be selectively inhibited in the method of the invention by interfering with the transcription of the gene encoding CYP2A6 using gene transfer methods such as targeted gene mutagenesis using allelic replacement, insertional inactivation, or deletion formation. For example, allelic gene exchange using nonreplicating or conditionally-replicating plasmids has been used widely for the mutagenesis of eukaryotes. Allelic exchange can be used to create a deletion of the CYP2A6 gene. Exemplary methods of making the alterations set forth above are disclosed by Sambrook et al (Molecular Cloning: A Laboratory Manual, 2d Ed., Cold Spring Harbor Laboratory Press, 1989).

CYP2B6 inhibitors may also be used in combination with inhibitors of CYP2A6 to provide an enhanced inhibitory effect. Inhibitors of CYP2B6 include one or more of the following (i) substances which inhibit CYP2B6 activity; or (ii) substances which inhibit transcription and/or translation of the gene encoding CYP2B6. CYP2B6 inhibitors may also be used alone to inhibit nicotine metabolism in an individual.

Substances which inhibit CYP2B6 activity include substances which specifically bind to CYP2B6 and thereby inhibit its activity. Examples of such substances include antibodies which are specific for CYP2B6 including for example, commercially available antibodies such as anti-CYP2B6 sold by Gentest Corporation, Woburn, Mass., U.S.A.

Substances which inhibit CYP2B6 activity also include substances selected from phenylethyl amines, diphenylbarbiturates, diethyl substituted barbiturates and hydantoins. In particular, diphenhydramine and its derivatives, including orphenadrine (The Merck Index, No. 6831), and derivatives or analogs of orphenadrine, and other antihistamines, anticholinergic substances such as cholines and analogs and derivatives thereof may be used as CYP2B6 inhibitors in various embodiments of the methods and compositions of the invention. Antibodies, such as polyclonal CYP2B1/2, polyclonal CYP2B1 and polyclonal CYP2B6 sold by Gentest Corporation, Woburn, Mass., U.S.A., also bind specifically to CYP2B6 such that they also inhibit the activity of CYP2B6.

Derivatives of orphenadrine which may be used in the methods and compositions of the invention include pharmaceutically acceptable salts, esters and complexes of orphenadrine including potassium and sodium salts, and amino acid, carbohydrate and fatty acid complexes. In one embodiment, suitable analogs of orphenadrine may be selected based upon their functional similarity to orphenadrine, including the ability to inhibit CYP2B6. Analogs of orphenadrine may also be selected based upon their three dimensional structural similarity to orphenadrine.

Substances which inhibit transcription and/or translation of the gene encoding CYP2B6 include a nucleic acid sequence encoding the CYP2B6 gene (see FIG. 2B, GenBank Accession No. HSP452B6 for the mRNA sequence of CYP2B6), or parts thereof (e.g., the region which is on either side of nucleotide 9 (ATG), and the sites 111, 274, 424, 585, 762, 904, 1092, and 1234 nt), inverted relative to their normal orientation for transcription—i.e., antisense CYP2B6 nucleic acid molecules. Such antisense nucleic acid molecules may be produced and introduced into cells using conventional procedures as described herein.

CYP2B6 may also be selectively inhibited in a method of the invention by interfering with the transcription of the gene encoding CYP2B6 using conventional gene transfer methods as discussed herein.

In preferred embodiments of the invention the CYP2B6 inhibitor employed is orphenadrine and derivatives or analogs of orphenadrine.

An inhibitor of CYP2A6 or CYP2B6 may be targeted to the enzyme using antibodies specific to an epitope of the enzyme. For example, bispecific antibodies may be used to target an inhibitor. The bispecific antibodies contain a variable region of an antibody specific for at least one epitope of CYP2A6 or CYP2B6, and a variable region of a second antibody which is capable of binding to an inhibitor. The bispecific antibodies may be prepared by forming hybrid hybridomas, using procedures known in the art such as those disclosed in Staerz & Bevan, (1986, PNAS (USA) 83: 1453) and Staerz & Bevan, (1986, Immunology Today, 7:241). Bispecific antibodies may also be constructed by chemical means using conventional procedures such as those described by Staerz et al., (1985, Nature, 314:628) and Perez et al., (1985 Nature 316:354), or by expression of recombinant immunoglobulin gene constructs.

The inhibitory activity of a particular substance identified herein or an analog or derivative thereof may be confirmed in experimental model systems, and in clinical studies, for example, the studies as outlined in the Examples herein below.

### 2. Screening Methods

As hereinbefore mentioned, the present invention provides a method of screening for a substance that regulates nicotine metabolism to cotinine in an individual comprising assaying for a substance which selectively (i) inhibits CYP2A6 activity, or (ii) inhibits transcription and/or translation of the gene encoding CYP2A6.

In an embodiment of the method of the invention, the method comprises:

(a) reacting a substrate of CYP2A6, in the presence of a test substance, under conditions such that CYP2A6 is capable of converting the substrate into a reaction product;

(b) assaying for reaction product, unreacted substrate or unreacted CYP2A6;

(c) comparing to controls to determine if the test substance selectively inhibits CYP2A6 and thereby is capable of regulating nicotine metabolism in an individual.

Substrates of CYP2A6 which may be used in the screening method of the invention for example include nicotine, coumarin, analogs thereof and derivatives thereof. The corresponding reaction products for nicotine and coumarin are cotinine, and 7-hydroxycoumarin, respectively.

CYP2A6 used in the method of the invention may be obtained from natural, recombinant, or commercial sources. For example CYP2A6 may be obtained by recombinant methods such as those described by Nesnow, S. et al., Mutation Research 1994; 324:93-102. Cells or liver microsomes expressing CYP2A6 may also be used in the method.

Conditions which permit the formation of a reaction product may be selected having regard to factors such as the nature and amounts of the test substance and the substrate.

The reaction product, unreacted substrate, or unreacted CYP2A6; may be isolated by conventional isolation techniques, for example, salting out, chromatography, electrophoresis, gel filtration, fractionation, absorption, polyacrylamide gel electrophoresis, agglutination, or combinations thereof.

To facilitate the assay of the reaction product, unreacted substrate, or unreacted CYP2A6; antibody against the reaction product or the substance, or a labelled CYP2A6 or substrate, or a labelled substance may be utilized. Antibodies, CYP2A6, substrate, or the substance may be labelled with a detectable marker such as a radioactive label, antigens that are recognized by a specific labelled antibody, fluorescent compounds, enzymes, antibodies specific for a labelled antigen, and chemiluminescent compounds.

The substrate used in the method of the invention may be insolubilized. For example, it may be bound to a suitable carrier. Examples of suitable carriers are agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc.

The insolubilized CYP2A6, substrate, or substance may be prepared by reacting the material with a suitable insoluble carrier using known chemical or physical methods, for example, cyanogen bromide coupling.

In another embodiment of the invention, a method is provided for screening for a substance that regulates nicotine metabolism to cotinine in an individual by inhibiting transcription and/or translation of the gene encoding CYP2A6 comprising the steps of:

(a) culturing a host cell comprising a nucleic acid molecule containing a nucleic acid sequence encoding CYP2A6 and the necessary elements for the transcription or translation of the nucleic acid sequence, and optionally a reporter gene, in the presence of a test substance; and

(b) comparing the level of expression of CYP2A6, or the expression of the protein encoded by the reporter gene with a control cell transfected with a nucleic acid molecule in the absence of the test substance.

A host cell for use in the method of the invention may be prepared by transfecting a suitable host with a nucleic acid molecule comprising a nucleic acid sequence encoding CYP2A6. A nucleic acid sequence encoding CYP2A6 may be constructed having regard to the sequence of the CYP2A6 gene (FIG. 2A) following procedures known in the art. Suitable transcription and translation elements may be derived from a variety of sources, including bacterial, fungal, viral, mammalian, or insect genes. Selection of appropriate transcription and translation elements is dependent on the host cell chosen, and may be readily accomplished by one of ordinary skill in the art. Examples of such elements include: a transcriptional promoter and enhancer or RNA polymerase binding sequence, a ribosomal binding sequence, including a translation initiation signal. Additionally, depending on the host cell chosen and the vector employed, other genetic elements, such as an origin of replication, additional DNA restriction sites, enhancers, and sequences conferring inducibility of transcription may be incorporated into the expression vector. It will also be appreciated that the necessary transcription and translation elements may be supplied by the native CYP2A6 gene and/or its flanking sequences.

Examples of reporter genes are genes encoding a protein such as galactosidase, chloramphenicol acetyltransferase, firefly luciferase, or an immunoglobulin or portion thereof such as the Fc portion of an immunoglobulin, preferably IgG. Transcription of the reporter gene is monitored by changes in the concentration of the reporter protein such as β-galactosidase, chloramphenicol acetyltransferase, or firefly luciferase. This makes it possible to visualize and assay for expression of CYP2A6 and in particular to determine the effect of a substance on expression of CYP2A6.

Suitable host cells include a wide variety of prokaryotic and eukaryotic host cells, including bacterial, mammalian, yeast or other fungi, viral, plant, or insect cells.

Protocols for the transfection of host cells are well known in the art (see, Sambrook et al. Molecular Cloning A Laboratory Manual, 2nd edition, Cold Sprinhg Harbor Laboratory Press, 1989). By way of example, Nanji M., et al., (1994) describe the expression of a cDNA encoding human CYP2A6 in a baculovirus system; Nesnow, S., et al. (1994) and Tiano H. F. et al, (1993) describe the expression of CYP2A6 from a retroviral vector in transformable C3H/10T1/2 mouse embryo fibroblasts; and Salonpaa, P., et al, (1993) describe the preparation of amphotropic recombinant retroviruses containing CYP2A6 using LXSN vector and PA317 packaging cells.

Host cells which are commercially available may also be used in the method of the invention. For example, the h2A3 (now known as h2A6) and h2B6 cell lines available from Gentest Corporation are suitable for the screening methods of the invention.

The above mentioned methods of the invention may be used to identify negative regulators of nicotine metabolism to cotinine in brain and liver thereby affecting conditions requiring regulation of nicotine metabolism. Identification and isolation of such regulators will permit studies of the role of the regulators in the regulation of nicotine metabolism to cotinine and permit the development of substances which affect this role, such as functional or non-functional analogs of the regulators. It will be appreciated that such substances will be useful as pharmaceuticals to modulate nicotine metabolism to cotinine as discussed herein.

The inhibitory activity of the substances identified by the methods of the invention may be confirmed in experimental model systems, and in clinical studies, for example the studies as outlined in the Examples herein below.

### 3. Compositions

Substances which inhibit nicotine metabolism to cotinine described in detail herein or substances identified using the methods of the invention which selectively inhibit CYP2A6 may be incorporated into pharmaceutical compositions. Therefore the invention provides a pharmaceutical composition for use in treating a condition requiring regulation of nicotine metabolism to cotinine comprising an effective amount of one or more substances which selectively inhibit CYP2A6, and/or a pharmaceutically acceptable carrier, diluent, or excipient. A method is also provided for treating a condition requiring regulation of nicotine metabolism to cotinine in a subject comprising administering to the subject an effective amount of one or more substances which selectively inhibit CYP2A6.

Conditions requiring regulation of nicotine metabolism to cotinine include nicotine use disorders—i.e., dependent and non-dependent tobacco use, and nicotine induced disorders—i.e., withdrawal. The conditions may develop with the use of all forms of tobacco (e.g., cigarettes, chewing tobacco, snuff, pipes, and cigars) and with prescription medications (e.g. nicotine gum, nicotine patch, spray, pulmonary inhalation or other forms). In particular, the pharmaceutical compositions and treatment methods of the invention may be used to diminish a subjects desire to smoke and thereby alter smoking behaviour. The pharmaceutical compositions and treatment methods of the invention may also be used together with other centrally active pharmaceutical compositions that modify smoking behaviour (e.g. bupropion (a.k.a. Wellbutrin®) in its various formulations), to decrease the dose of the centrally active composition or to increase its effectiveness in the treatment of tobacco dependence.

The compositions and treatment methods of the present invention by regulating nicotine metabolism in an individual are highly effective. The methods and compositions maintain the behavioural components of smoking and modify them by reducing nicotine metabolism to cotinine. An individual with reduced nicotine metabolism following administration of a composition of the present invention, will alter smoking behaviour and smoke exposure because of modification of nicotine requirements. The methods and compositions of the invention show patterns of reduction, more sustained abstinence, and lower tobacco smoke exposure than obtained with prior art methods in particular those using nicotine deprivation.

The behavioural component of smoking is particularly important in some groups of individuals, and thus the methods and compositions of the invention in modifying and maintaining behavioural components may be particularly useful in reducing smoking in those individuals. For example, it has been found that behavioural components are significant in tobacco use by women. The present invention permits the development of behavioural learning on an individual/or group basis.

The compositions and treatment methods of the invention are also particularly suited to regulate nicotine metabolism in individuals or populations having high levels of CYP2A6. For example, Caucasians in North America have high levels of CYP2A6. An individual or population having a high level of CYP2A6 can be identified using our methods for measuring CYP2A6.

The compositions and methods of the invention also have the advantage of individualization and flexibility in treatment duration. The compositions and treatment methods are particularly suitable for severely dependent individuals, previous treatment failures, individuals unable to accept the current approach of complete cessation, treatment/prevention of relapse, or concurrent treatment with other methods such as the nicotine patch. It is expected that the compositions and treatments of the invention will decrease the doses of nicotine patch and all other forms of nicotine replacement therapies that are needed and will prolong the duration of action of the therapy and/or enforce their effectiveness in the treatment of tobacco dependence.

The methods and compositions of the invention in treating individuals with nicotine use disorders and nicotine-induced disorders are also useful in the treatment and prophylaxis of diseases or conditions, including nicotine-related disorders such as opioid related disorders; proliferative diseases; cognitive, neurological or mental disorders; and other drug dependencies in the individuals. Examples of such underlying diseases or conditions include malignant disease, psychosis, schizophrenia, Parkinson's disease, anxiety, depression, alcoholism, and opiate dependence.

The methods and compositions of the invention may also be used in the prophylaxis and treatment of individuals having a condition which requires a reduction in CYP2A6 or CYP2B6. For example, CYP2A6 is known to metabolize several procarcinogens such as NNK (Crespi et al., 1991), aflaxtoxin B1 (Yun et al., 1991); hexamethylphosphoramide (Ding et al., 1988), and nitrosodimethylamine (Davies et al., 1989; Fernandez et al., 1995). Therefore, inhibitors of CYP2A6 may be useful in the prophylaxis and treatment of malignant diseases.

The pharmaceutical compositions of the invention contain substances which selectively inhibit CYP2A6 described in detail herein or substances identified using the methods of the invention. The active substances can be administered alone, but are generally administered with a pharmaceutical carrier etc. (see below), selected on the basis of the chosen route of administration and standard pharmaceutical practice.

The dosage administered will vary depending on the use and known factors such as the pharmacodynamic characteristics of the particular substance, and its mode and route of administration; age, health, and weight of the individual recipient; nature and extent of symptoms, kind of concurrent treatment, frequency of treatment, and the effect desired.

In some instances, instead of increasing the dosage of a compound, the kinetics of inhibition created by certain chemical compounds can be altered or enhanced by adding to the treatment protocol a second inhibitor to a substance (e.g., enzyme) that is capable of inhibiting the metabolism of the CYP2A6 inhibitor. By adding such a second inhibitor, the quantity of the CYP2A6 inhibitor will be maintained thus prolonging the beneficial effect of maintaining an elevated plasma concentration of nicotine. The use of such a second inhibitor is very beneficial since it facilitates treatment of individuals by maintaining substantially constant nicotine levels and acting locally on the kinetics of the CYP2A6 inhibitor. By using this approach, large dosages of centrally active compounds can be avoided.

Similarly, preexposure of an individual to an inhibitory substance sometimes can result in an inhibitory effect that will outlast the presence of the drug in the plasma or that will have a persistent effect in the individual despite the inhibitor's half life in the plasma. This phenomenon caused by preincubation or preexposure of an inhibitory substance can help increase the dose interval at which a dosage of the substance must be administered, decrease the chronic dose or enhance CYP2A6 inhibition. Furthermore, preexposure of an individual to one inhibitory substance can subsequently decrease the needed dose of a second inhibitor.

The appropriate dosage of a substance which selectively inhibits CYP2A6 is dependent upon the amount of CYP2A6 that is present in an individual's body. This amount is in turn dependent upon whether the individual contains two mutant alleles, one mutant allele or no mutant alleles at the CYP2A6 gene locus. In Example 7, we confirmed that such variations can exist in the genetic material of a population. It is, therefore, an aspect of this invention to provide a method for determining the CYP2A6 activity in an individual containing two mutant alleles, one mutant allele or no mutant alleles at a gene locus for the CYP2A6 gene, the method comprising the steps of:

(a) assaying a bodily sample containing deoxyribonucleic acid (i.e. a “DNA-containing bodily sample”) from the individual to determine whether the individual contains two mutant alleles, one mutant allele or no mutant alleles at the CYP2A6 gene locus;

(b) determining the amount of CYP2A6 present in the individual; and

(c) correlating the results of assaying in step (a) and the amount of CYP2A6 in step (b) to determine an appropriate dosage for that individual of a substance which (i) selectively inhibits CYP2A6 activity, or (ii) selectively inhibits transcription and/or translation of the gene encoding CYP2A6.

The individual recipient may be any type of mammal, but is preferably a human. Generally, the recipient is an individual having a CYP2A6 genotype associated with an active form of the enzyme. The CYP2A6 genotype of an individual and the existence of an active CYP2A6 enzyme in an individual may be determined using procedures described herein. For example, coumarin 7-hydroxylation has been used to measure CYP2A6 activity (Cholerton et al., 1992; and Rautio et al., 1992). As discussed above, the methods and compositions of the invention may be preferably used in individuals or populations having high levels of CYP2A6, or in individuals where the behavioural components of smoking are significant.

For use in the treatment of conditions requiring regulation of nicotine metabolism to cotinine, by way of general guidance, a daily oral dosage of an active ingredient such as coumarin or methoxsalen can be about 0.1 to 80 mg/kg of body weight, preferably 0.1 to 20, more preferably 0.2 to 3 mg/kg of body weight. Ordinarily a dose of 0.5 to 50 mg/kg of coumarin or methoxsalen per day in divided doses one to multiple times a day, preferably up to four times per day, or in sustained release form is effective to obtain the desired results. In accordance with a particular regimen, coumarin or methoxsalen is administered twice daily for one to four days. While standard interval dose administration may be used the compositions of the invention may be administered intermittently prior to high risk smoking times, e.g., early in the day and before the end of a working day.

The substances for the present invention can be administered for oral, topical, rectal, parenteral, local, inhalant or intracerebral use. In an embodiment of the invention, the substances are administered in intranasal form via topical use of suitable intranasal vehicles, or via transdermal routes, using forms of transdermal skin patches known to those of ordinary skill in that art. To be administered in the form of a transdermal delivery system, the dosage administration will be continuous rather than intermittent throughout the dosage regimen. The substances can also be administered by way of controlled or slow release capsule system and other drug delivery technologies.

In the methods of the present invention, the substances described in detail herein and identified using the method of the invention form the active ingredient, and are typically administered in admixture with suitable pharmaceutical diluents, excipients, or carriers suitably selected with respect to the intended form of administration, that is, oral tablets, capsules, elixirs, syrups and the like, consistent with conventional pharmaceutical practices.

For example, for oral administration in the form of a tablet or capsule, the active substances can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral active substances can be combined with any oral, non-toxic, pharmaceutically acceptable inert carrier such as ethanol, glycerol, water, and the like. Suitable binders, lubricants, disintegrating agents, and colouring agents can also be incorporated into the dosage form if desired or necessary. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Suitable lubricants used in these dosage forms include sodium oleate, sodium stearate, magnesium stearate, sodium benzoate, sodium acetate, sodium chloride, and the like. Examples of disintegrators include starch, methyl cellulose, agar, bentonite, xanthan gum, and the like.

Gelatin capsules may contain the active substance and powdered carriers, such as lactose, starch, cellulose derivatives, magnesium stearate, stearic acid, and the like. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract. Liquid dosage forms for oral administration may contain colouring and flavouring agents to increase patient acceptance.

Water, a suitable oil, saline, aqueous dextrose, and related sugar solutions and glycols such as propylene glycol or polyethylene glycols, may be used as carriers for parenteral solutions. Such solutions also preferably contain a water soluble salt of the active ingredient, suitable stabilizing agents, and if necessary, buffer substances. Suitable stabilizing agents include antioxidizing agents such as sodium bisulfate, sodium sulfite, or ascorbic acid, either alone or combined, citric acid and its salts and sodium EDTA. Parenteral solutions may also contain preservatives, such as benzalkonium chloride, methyl- or propyl-paraben, and chlorobutanol.

The substances described in detail herein and identified using the methods of the invention can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Substances described in detail herein and identified using the methods of the invention may also be coupled with soluble polymers which are targetable drug carriers. Examples of such polymers include polyvinylpyrrolidone, pyran copolymer, polyhydroxypropylmethacrylamide-phenol, polyhydroxyethylaspartamidephenol, or polyethyleneoxide-polylysine substituted with palmitoyl residues. The substances may also be coupled to biodegradable polymers useful in achieving controlled release of a drug. Suitable polymers include polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels. The substances can also be affixed to rigid polymers and other structures such as fullerenes or Buckeyballs.

Pharmaceutical compositions suitable for administration contain about 1 milligram to 1500 milligrams of active substance per unit. In these pharmaceutical compositions, the active ingredient will ordinarily be present in an amount of about 0.5-95% by weight based on the total weight of the composition.

Suitable pharmaceutical carriers and methods of preparing pharmaceutical dosage forms are described in Remington's Pharmaceutical Sciences, Mack Publishing Company, a standard reference text in this field.

More than one substance described in detail herein or identified using the methods of the invention may be used to regulate metabolism of nicotine to cotinine. In such cases the substances can be administered by any conventional means available for the use in conjunction with pharmaceuticals, either as individual separate dosage units administered simultaneously or concurrently, or in a physical combination of each component therapeutic agent in a single or combined dosage unit. The active agents can be administered alone, but are generally administered with a pharmaceutical carrier selected on the basis of the chosen route of administration and standard pharmaceutical practice as described herein.

The combination of an CYP2A6 inhibitor (e.g., coumarin, methoxsalen), and a CYP2B6 inhibitor (e.g., orphenadrine) enhances inhibition of nicotine metabolism to cotinine. Thus, a preferred embodiment of the invention provides a method for treating conditions requiring regulating nicotine metabolism to cotinine comprising administering an effective amount of a CYP2A6 inhibitor and an effective amount of a CYP2B6 inhibitor to selectively inhibit nicotine metabolism to cotinine. In a preferred embodiment of the invention, the CYP2A6 inhibitor is methoxsalen or an analog or derivative thereof, and the CYP2B6 inhibitor is orphenadrine, or an analog or derivative thereof. The inhibitors may be administered concurrently, separately or sequentially. The doses of the CYP2A6 inhibitor and the CYP2B6 inhibitor are each selected so that each inhibitor alone would not show a full effect. The effective doses are those which are approximately the minimum doses adequate for enhanced inhibition of nicotine metabolism to cotinine. Pharmaceutical compositions containing combinations of CYP2A6 and CYP2B6 inhibitors may be prepared, and administered as described herein for the compositions containing CYP2A6 inhibitors. The pharmaceutical compositions preferably contain methoxsalen or an analog or derivative thereof, and orphenadrine, or an analog or derivative thereof, in concentrations of 1 to 1500 mg, and 25 to 400 mg, respectively.

The recognition by the present inventors that CYP2A6 is the major nicotine metabolizing enzyme in human livers suggests that the enzyme can be assayed in an individual to determine the individual's risk of developing tobacco dependence. Determination of CYP2A6 levels may also be used to select and monitor in an individual appropriate conventional nicotine replacement therapies such as the nicotine patch and nicotine gum. It is unlikely that conventional nicotine replacement therapies (e.g. nicotine gum, nicotine patch, spray, pulmonary inhalation or other forms) will have a high success outcome if an individual has high levels of CYP2A6. Conversely, if an individual has very low levels of CYP2A6, administering nicotine at high dosages will likely result in increased toxicity, and side effects.

The following non-limiting examples are illustrative of the present invention:

### EXAMPLE 1

### Role of CYP2D6 in Nicotine Metabolism

The following materials and methods were utilized in the investigations outlined in Example 1:

### Materials and Methods:

### Biological Samples

Human livers. The characteristics and sources of the K series livers used in this study were described previously (Campbell et al., 1987; Tyndale et al., 1989). The K series livers were obtained from Dr. T. Inaba, University of Toronto. The L series livers were obtained from Dr. E. Roberts from the Hospital for Sick Children (Toronto, ON, Canada), and consisted of partial livers obtained from liver donors.

CYP2D6 yeast. Microsomal preparations of CYP2D6 expressed in yeast (aH22/peltl cells) and control yeast (AH22/pMA91 cells) were provided by Dr. M. S. Lennard, University of Sheffield, U.K. Inunochemical and catalytic assays have indicated that cytochrome P450 was undetectable in microsomes prepared from the control yeast, and that the enzyme activity in microsomes prepared from CYP2D6 expressing yeast was predominantly due to CYP2D6 (Ellis et al., 1992). Microsomal protein concentrations were determined by the BSA assay kit (Pierce Chemical Co., Rockford, Ill., USA).

Drugs and Chemicals. Dextromethorphan hydrobromide, (S)-nicotine, (S)-cotinine, quinidine, ketamine, cumene hydroperoxide, and NADPH were obtained from Sigma Co. (St. Louis, Mo., USA). Dextrorphan, methoxymorphinan, and hydroxymorphinan were provided by Hoffmann-La Roche Inc., Nutley, (N.J., USA). Budipine was obtained from Byk Gulden Pharmazeutika, Konstanz, Germany.

Microsome Preparation. The partial livers (˜2 grams) from 30 humans were thawed on ice, then minced in two volumes of cold 1.15% KCl. The samples were homogenized by applying ten strokes of a Teflon pestle powered by a Black and Decker electric drill. Each liver homogenate was then subjected to a centrifugation of 9000 g for 20 min. at 4° C. in a Sorvall RC2-B. The supernatant, which contains cytosol and microsomes, was decanted and centrifuged at 100,000 g for 60 min. at 4° C. in a Sorvall Combi Plus Ultraspeed Centrifuge. The resulting microsomal pellet was resuspended in 1.15% KCl and centrifuged again at 100,000 g for 60 min. at 4° C. for further purification. The microsomal pellet was resuspended in a 2:1 vol/wt solution of 1.15% KCl and stored in a Form a Scientific freezer at −70° C.

Protein Determination. Protein concentration of the microsomal samples were determined with a Pierce BCA protein assay kit (Pierce Chemical Co., Rockford, Ill., USA), using the bovine serum albumin standard (BSA) solution provided. Samples, in duplicate, were diluted with H₂0 to a concentration in the range of the BSA standards. 100 μl of each sample, or BSA standard, was added to 2 mls of Pierce Working Reagent. This reagent solution contained 50 parts Reagent A to 1 part Reagent B. Samples were vortexed and then incubated in a shaking water bath for 30 min. at 37° C. Absorbence of each sample was then measured at 562 nm against a blank vial.

Analytical Methods for Dextromethorphan assay. Incubation conditions of this assay were adapted from those of Otton et al., (1983).

Dextromethorphan to dextrorphan kinetics: Dextromethorphan to dextrorphan kinetics was measured as a function of protein concentration and time. Dextromethorphan at a concentration of 5 μM was incubated with 0.025, 0.05, 0.1, 0.2, 0.3, 0.4, and 0.5 mg protein/ml, with 0.8 mM NADPH in 0.2 M phosphate buffer (pH 7.4). The incubation mixture was comprised of 125 μl phosphate buffer (pH 7.4), 50 μl microsomal protein, 50 μl dextromethorphan, and 25 μl NADPH for a total volume of 250 μl. Incubations were carried out at 37° C. for 30 min. in a shaking water bath, and were terminated with the addition of 10 μl perchloric acid. Budipine was used as an internal standard. Samples were then centrifuged at 3000 rpm for 5 min., and 30 μl of the supernatant was analyzed by HPLC. Results revealed that dextrorphan production was linear from 0.025 to 0.5 mg protein/ml throughout a 30 min. incubation. Apparent Kₘ, and Vₘₐₓ values were determined by incubating dextromethorphan at 1, 2.5, 5, 10, 50, and 75 μM concentrations in duplicates, with a microsomal protein concentration of 0.125 mg/ml, for 30 minutes at 37° C.

HPLC. The HPLC system (Hewlett Packard) consists of a 1050 series pump and autosampler, connected to a HP 3396 series II integrator. A CSC-Spherisorb-Phenyl (5 um, 4.6 mm×25 cm) column and a mobile phase consisting of 10 mM potassium phosphate buffer containing 1 mM Heptanesulfonic acid, pH 3.8, and acetonitrile (80:20 v/v) was used, with a flow rate set at 1.7 ml/min. Dextromethorphan and the various metabolites in the incubation samples were measured as described by Broley et al. (1989), except that the excitation and emission wavelengths were set at 195 nm and 280 nm, respectively for a higher sensitivity. Dextrorphan calibration curves were linear from 0 to 120 pmoles, with the lowest detectable level of 5 pmoles for dextrorphan. The coefficient of variation within-day was 2.7 and 2.0% (n=5), for 0.25 and 0.5 pmoles/ml injections of dextrorphan respectively. The coefficient of variation between-day was 6.5 and 9.6% (n=6), for 0.125 and 0.5 nmoles/ml concentrations of dextrorphan respectively.

### Analytical Methods for the Nicotine Assay

Incubation: Microsomes were removed from a −70° C. freezer and thawed on ice. Incubation mixtures generally contained 100 μl (S)-nicotine, 100 μl NADPH (1 mM final), 200 μl human liver microsomes (0.5 mg/ml), 20 μl Wistar rat liver cytosol, 200 μl potassium phosphate buffer (pH 7.4, 40 μM final), diluted to a 1 ml final volume with 1.15% KCl. The reaction was initiated with the addition of NADPH and placing the samples at 37° C. The mixtures were incubated in a polypropylene conical 10 ml tubes then placed in a Precision Scientific Shaker Bath (Model 50) at 37° C. for 45 minutes. The reaction was stopped by adding 100 μl of 20% Na₂CO₃ (pH 11.4). Nicotine to cotinine kinetic studies were performed by incubating 1, 5, 10, 50, 100, and 200 μM (S)-nicotine with 0.5 mg/ml microsomal protein for 45 min, in the presence of 20 μl rat liver cytosol, 1 mM NADPH, in 40 mM phosphate buffer (pH 7.4). The reaction was started by adding NADPH. The incubation mixture comprised of kinetic parameters were calculated by use of computer program Enzfitter (Robin J. Leatherbarrow, 1987). The data were fit by one-site Michaelis-Menten rate equation.

Extraction: After basification with Na2CO3, 10 μl of ketamine (the internal standard) was added to each sample. Ethyl acetate (3 mls) was added for extraction purposes. The samples were vortexed vigorously for 5 min., followed by centrifugation for 5 min. at 3000 rpm in an GLC-2B centrifuge. The organic layer (top) was pipetted to a separate 10 ml conical tube, which contained 400 μl of 0.01 N HCl. The samples were vortexed again for 5 min. and centrifuged at 3000 rpm for 5 min. The organic layer was then discarded, and the remaining aqueous layer was dried under nitrogen at 37° C. for 30 min. to remove any remaining organic solvent. 30 μl of each sample was then subjected to High Performance Liquid Chromatography (HPLC) analysis.

HPLC: Separation of nicotine and metabolites was achieved by using a CSC Spherisorb-Hexyl column (15×0.46 cm) and a mobile phase consisting of 20% acetonitrile and 80% 20 mM potassium phosphate, pH 4.6, containing 1 mM octanesulfonic acid was used. The separation was performed with isocratic elution at a flow rate of 1 ml/min. The retention times for cotinine, nicotine and ketamine were 3.5, 4.2, and 7.0 minutes respectively. The minimum detectable limit in the system was 300 pmoles of cotinine per ml of incubation mixture. Within and between day variations were found to be below 10% (n=6). The HPLC system consisted of a Hewlett Packard 1090 solvent delivery system linked to a 1050 series UV detector. The UV detector was set at 210 nm to optimize for cotinine detection.

Data Analysis. The UV absorbence data was transferred to a Hewlett-Packard Chemstation. The three peaks of interest were cotinine, nicotine, and ketamine (internal standard). The heights of the respective peaks were used to determine peak height ratios. Specifically, the cotinine peak height ratio was determined by measuring the ratio between the height of the cotinine peak to the ketamine peak height.

$$Peak Height Ratio ( PHR ) = \frac{cotinine peak height}{ketamine peak height}$$

The peak height ratios were used to analyze the relative amount of cotinine production, and to determine the specific concentration of cotinine present in each sample incubation. This was achieved by producing standard curves during each session of data collection.

Standard curves were produced by injecting various concentrations of cotinine into the HPLC. Cotinine amounts typically included 1.25, 2.5, 5.0, and 10.0 nmole concentrations. 10 μl of ketamine, at a concentration of 0.25 mg/ml, was added to each sample. The standard curve enabled any given PHR obtained from a given sample to be converted to its respective concentration of cotinine in nmoles.

Within-day and Between-day variations. The within-day variation and between day variation of the assay were calculated for two concentrations of cotinine. Standard solutions contained 2.5 nmoles and 5.0 nmoles of cotinine per ml of incubation mixture. Samples contained 40 mM phosphate buffer, and 1.15% KCl. They were subjected to similar extraction and evaporation procedures as mentioned above. For cotinine concentrations of 2.5 nmoles and 5.0 moles/ml the within day coefficients of variations were 3.1% and 2.3% respectively. The between-day variation were 7.2% and 8.4%. A coefficient of variation (CV) less than 10% was deemed acceptable.

Cytosol assay. Cytosolic fractions from livers of 4 male Wistar rats were used as the source of aldehyde oxidase. Since the metabolism of nicotine to cotinine is a two step reaction involving an imminium ion intermediate, it was necessary to make the cytochrome P450 oxidation of nicotine the rate determining step. This was performed by adding excess aldehyde oxidase. Cotinine production increases and then plateaus with the addition of increasing amounts of rat cytosol. It was determined that 20 μl of cytosol would be used as the source of aldehyde oxidase. The rat cytosol had no intrinsic nicotine oxidase activity.

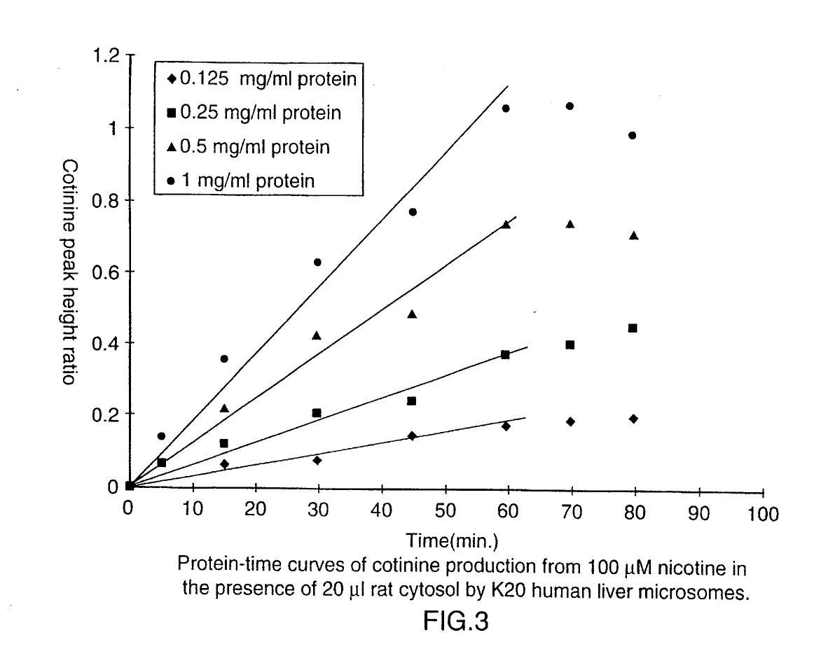

Protein-time assay. Protein concentrations of 0.125, 0.25, 0.5, and 1 mg protein/ml from the K20 liver microsome sample were incubated at 37° C., with 20 μl rat liver cytosol, 1 mM NADPH, in 40 mM phosphate buffer (pH 7.4) across several time intervals. Results are presented in FIG. 3. These results show that cotinine formation is linear at a protein concentration of 0.125 to 0.5 mg/ml for a 45 min. incubation. Cotinine formation was also dependent on NADPH concentrations. A NADPH concentration of 1 mM was determined to be optimal for the above experimental conditions.

Quinidine and coumarin inhibition of cotinine formation. Nicotine was incubated with 0.5 mg/ml microsomal protein from K20 human liver. Incubations included 1 mM NADPH, 20 μl rat liver cytosol, 40 mM phosphate buffer (pH 7.4), and were carried out for 45 minutes at 37° C. Inhibition studies included adding 100 μM quinidine, 100 μM coumarin, 100 μM of quinidine and coumarin, with 60 μM (S)-nicotine.

Incubation of nicotine in yeast expressing CYP2D6. Incubation conditions using CYP2D6 expressed in yeast supported by cumene hydroperoxide (CuOOH), were essentially those of Zanger et al., (1988) and Wu (1993). Basically, CuOOH (80% in cumerol, Sigma) was first diluted to a concentration of 40 mM in 50% methanol in H₂O (v/v) and then to 375 μM in 0.3 M potassium phosphate buffer, pH 7.4. 200 μl of this solution was added to 100 μl nicotine (100 μM final) and 20 μl of rat liver cytosol, in a final volume of 1 ml (final CuOOH concentration of 75 μl). The incubation was initiated by the addition of 200 μl of yeast protein (0.3 mg/ml final) and was for 20 min. at room temperature. Incubations were carried out in a shaking water bath, at 37° C., for 120 min. All reactions were stopped with the addition of 100 μl 20% Na₂CO₃ (pH 11.4).

### Results

Dextromethorphan to dextrorphan metabolism in human liver microsomes. The kinetics of dextrorphan formation were determined using a non-linear least squares algorithm in which the data were weighted by the reciprocal of the rate of metabolism and were fit by one or two site Michaelis-Menten kinetic models. The L11 liver sample displays a high affinity enzyme kinetics, while the L3 liver sample displays both high and low affinity enzyme kinetics. Low affinity enzyme sites were observed in 2 out of the 11 livers, while the remaining 9 livers had only a high affinity enzyme site with Kₘ and Vₘₐₓ values (mean±SD, n=9) of 5.79±2.01 μM and 10.03±6.53 nmoles/mg protein/hr, respectively. Since the Kₘ value was approximately 5 μM, a 5 μM dose of dextromethorphan was used in incubations with 30 human liver microsomes. The rate of dextrorphan formation was used as a measure of CYP2D6 activity. PCR studies revealed two poor metabolizer genotypes (b/b) for CYP2D6 mediated reactions (L18, and L19), four heterozygote extensive metabolizers (wt/b: L26, L27, L61 and L63), with the remaining livers displaying the extensive metabolizer genotype (wt/wt).

### Nicotine to Cotinine Kinetics

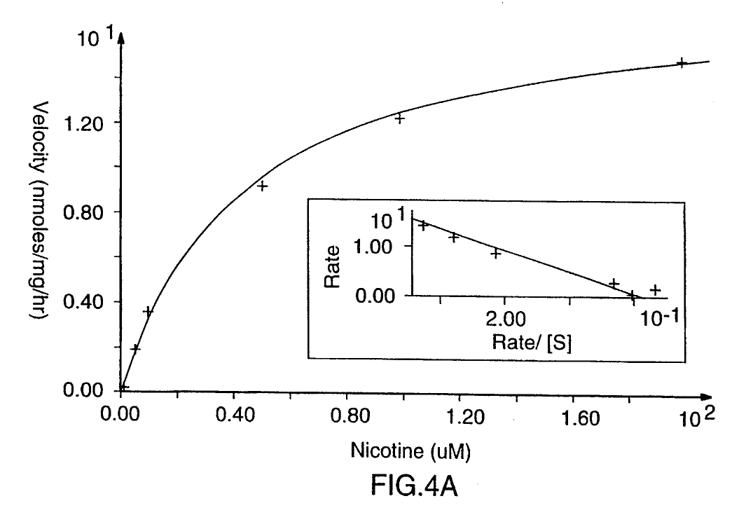

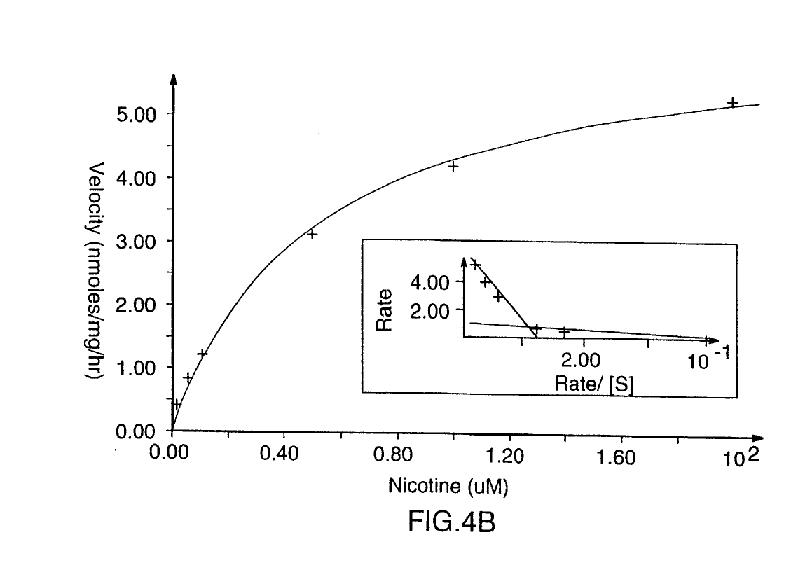

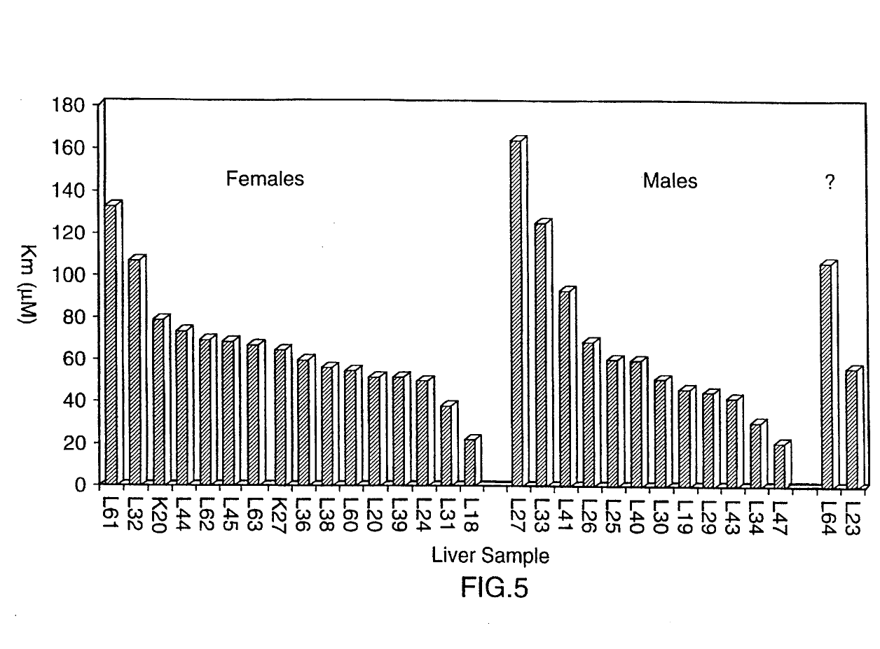

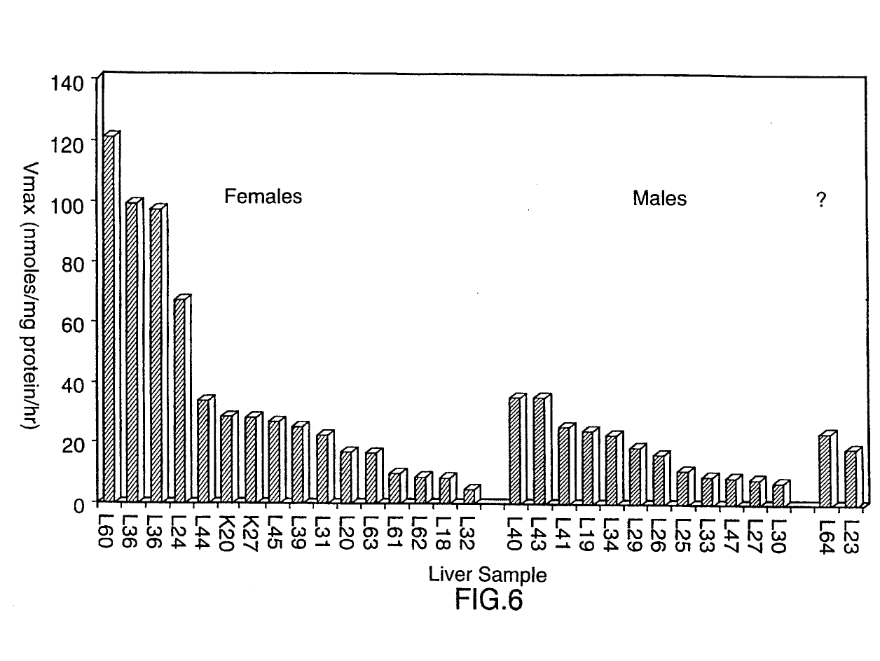

Kₘ and Vₘₐₓ values for nicotine to cotinine kinetics were calculated for all 30 human liver microsomes. Sample Michaelis-Menten curves for nicotine to cotinine kinetics are shown in FIG. 4. These graphs show livers which display one site or multiple site enzyme kinetics. FIG. 5 compares the respective Kₘ values across all 30 samples. These figures were segregated into male and female liver donors so that sex differences could be examined. The mean Kₘ value for all 30 livers is 66.6±31.8 mM (mean±SD). Vₘₐₓ results revealed marked inter-individual variations in cotinine formation (FIG. 6). Four human livers appeared to have very high rates of cotinine formation. The Vₘₐₓ values between males and females was significantly different (p=0.07), but not when the four high female Vₘₐₓ values were removed (p=0.78), as determined by the students t-test. There is approximately a 30 fold difference in the Vₘₐₓ values of cotinine formation between the L32 and L60 liver microsome samples. The mean Vₘₐₓ value for all 30 livers is 28.9±28.9 nmoles/mg protein/hr (mean±SD).

### Correlation Between CYP2D6 Activity and Cotinine Formation

CYP2D6 as measured by dextromethorphan metabolism to dextrorphan was compared to nicotine to cotinine Vₘₐₓ values across the 30 human livers. Specifically, the rate of dextrorphan formation was used as a measure of CYP2D6 activity. Results revealed no correlation (r=0.21, p=0.27), between CYP2D6 activity and cotinine formation.

### Inhibition of Cotinine Formation

Quinidine, which is a specific inhibitor of the CYP2D6 enzyme, had some inhibitory effect on cotinine formation. Quinidine at 100 μM (1000 times greater than its Kᵢ value for inhibiting dextromethorphan to dextrorphan metabolism by CYP2D6; Kerry et al., 1994) inhibited cotinine formation by approximately 20%. In the presence of 100 μM coumarin, cotinine formation was inhibited by over 80%, with little additional inhibition when quinidine was added in combination with coumarin.

Nicotine Metabolism with Yeast Expressing CYP2D6

Nicotine incubations with yeast expressing CYP2D6 and yeast controls showed no difference in cotinine peak height ratios. The possibility of inactive CYP2D6-expressing yeast was investigated; p-methoxy-amphetamine, methamphetamine, and dextromethorphan, substrates for CYP2D6, were all oxidized by the CYP2D6-expressing yeast. Nicotine incubations in yeast were performed simultaneously with para-methoxyamphetamine incubations.

### Discussion:

The metabolism of dextromethorphan and nicotine was studied using a bank of 30 human liver microsomes. Dextromethorphan metabolism to dextrorphan revealed typical Michaelis-Menten kinetics with some livers displaying a low affinity site. The apparent high affinity Kₘ value was 5.79±2.01 (mean±SD). Thus, 5 μM of dextromethorphan was incubated with 30 human liver microsomes to access CYP2D6 activity in each liver. Nicotine to cotinine metabolism was also investigated in the same 30 human livers. This required the development of a novel assay which is described above. Since the metabolism of nicotine to cotinine is a two step reaction, excess amounts of aldehyde oxidase was added to each incubation, such that the CYP oxidation step was rate determining. The formation of cotinine followed Michaelis-Menten kinetics with apparent Kₘ and Vₘₐₓ values (mean±SD) of 66.7±31.8 μM and 28.9±28.9 nmoles/mg protein/hr, respectively.

Because of recent studies (Cholerton et al., 1994) which implicated a role for the CYP2D6 poor metabolizer phenotype of debrisoquine to the poor metabolism of nicotine, CYP2D6's role was addressed. The first set of experiments were aimed at drawing a potential correlation between CYP2D6 activity and nicotine oxidation to cotinine among 30 human liver microsomes. Dextromethorphan was used as the probe drug for CYP2D6 activity. Results revealed that liver microsomes which displayed low rates of dextrorphan formation was closely related to CYP2D6 genotyping studies. Dextrorphan rates of formation were plotted against nicotine to cotinine Vₘₐₓ values. No correlation was found between CYP2D6 activity and nicotine to cotinine metabolism (r=0.21, n=30) suggesting that CYP2D6 is not a major enzyme involved in nicotine metabolism.

Correlation studies are not a conclusive form of evidence thus supplementary studies were performed. Quinidine, a specific CYP2D6 inhibitor, was tested at 0.1, 1.0, 10 and 100 μM. At 0.1 and 1.0 μM, concentrations specific for CYP2D6 inhibition, no inhibition of cotinine formation was observed. At 10 and 100 μM, 100-1000 times above the Kᵢ value for CYP2D6 (Kᵢ˜100 nM; Kerry et al., 1994), 8 and 25% inhibition of cotinine formation occurred, respectively. In contrast coumarin, a specific CYP2A6 substrate, inhibited cotinine formation by over 80% at the same concentration. Stronger evidence excluding CYP2D6 from nicotine metabolism comes from the cDNA-expression work. Nicotine incubations with lysed lymphoblast cells expressing CYP2D6 or with yeast microsomes expressing CYP2D6 cDNA, both failed to metabolize nicotine to cotinine, but were able to metabolize the CYP2D6 substrate dextromethorphan (5 μM). Further supporting evidence comes from Wu, 1993 who showed that nicotine did not inhibit dextromethorphan metabolism by CYP2D6 in human liver microsomes.

### EXAMPLE 2

### Role of CYP2A6 and CYP2B6 in Nicotine Metabolism

Since the CYP2D6 enzyme appears not to be involved in nicotine to cotinine metabolism, an investigation of the role of other cytochromes P450 was undertaken. In particular the importance of CYP2A6 in contributing to inter-individual differences in nicotine metabolism was accessed in vitro. CYP2A6, heterogously expressed in human lymphoblastoid cells, has one of the highest activities in the conversion of nicotine to cotinine, second only to CYP2B6 (McCracken et al, 1992).

The following materials and methods were utilized in the investigations outlined in Example 2:

### Materials and Methods:

Human liver microsomes. The same 30 human liver samples were used in this study as were used in Example 1.

Drugs and chemicals. (S)-Nicotine, (S)-cotinine, NADPH, Tris-HCl, octanesulfonic acid, troleandomycin, orphenadrine, and ketamine were obtained from Sigma Chemical Co. (St. Louis, Mo., USA). 7-methoxycoumarin, 7-methylcoumarin, and 7-ethoxycoumarin were purchased from Aldrich (St. Louis, Mo., USA). Coumarin and ethyl acetate were obtained from Calcdon (Georgetown, ON, Canada). Potassium phosphate was purchased from Mallinchrodt (Mississauga, ON, Canada). Antibodies were purchased from Gentest Corp. (Woburn, Mass., USA).

Chemical inhibition studies. Extensive chemical inhibition studies consisted of incubating 100 μM (S)-nicotine with 150 μM concentrations of coumarin, orphenadrine, troleandomycin, and coumarin with orphenadrine in combination, with all 30 human liver microsomes, in duplicate. To maximize inhibition, and reduce the loss due to its own metabolism, a concentration of 150 μM was chosen for each specific inhibitor. From the kinetic studies this inhibitor concentration is approximately 2 times the mean Kₘ value for nicotine to cotinine metabolism. The nicotine concentration was set at 100 μM which was the concentration that was approaching the Vₘₐₓ for cotinine formation. This concentration was chosen to maximize the contributions of each cytochrome P450 involved in nicotine metabolism, since each enzyme would likely have varying affinities for nicotine. Incubation conditions were similar to that mentioned in the previous example. Once coumarin was shown to inhibit nicotine metabolism, coumarin analogs were incubated with nicotine. High and low concentrations (10 and 100 AM) of coumarin, 7-methylcoumarin, 7-methoxycoumarin, and 7-ethoxycoumarin were incubated with 50 μM nicotine in K27 human liver microsomes.

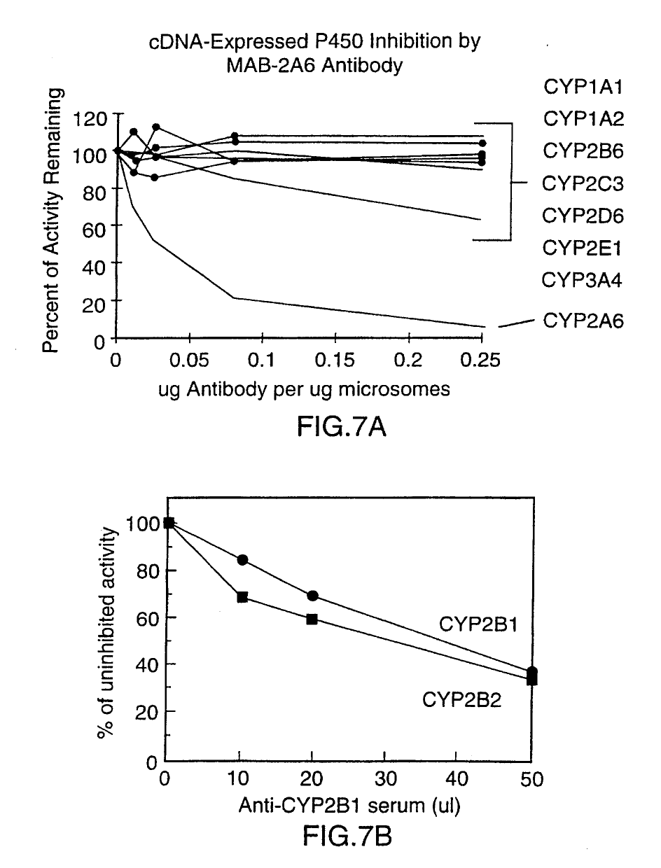

Immunochemical inhibition studies. Immunoinhibition experiments consisted of incubating 0.5 mg/ml K27 liver microsomes with a CYP2A6 monoclonal antibody (MAB-2A6) and a CYP2B1 (anti-rat CYP2B1) polyclonal antibody. Antibodies and microsomes were preincubated on ice for 30 minutes followed by the addition of 100 μM nicotine, 1 mM NADPH, and 20 μl rat cytosol in 25 mM Tris-HCl buffer. Antibody concentrations were chosen based on immunoinhibition information provided by Gentest Corp. FIG. 7 shows the potency and specificity of the MAB-2A6 and anti-rat CYP2B1 for their respective enzymes. Gentest Corp. stated that MAB-2A6 inhibited over 95% 2A6 activity, at 0.25 mg antibody/μg microsomal protein. They used coumarin hydroxylation as a measure of 2A6 activity. They also showed that anti-rat 2B1 cross-reacts with the human CYP2B6 enzyme to inhibit its activity.

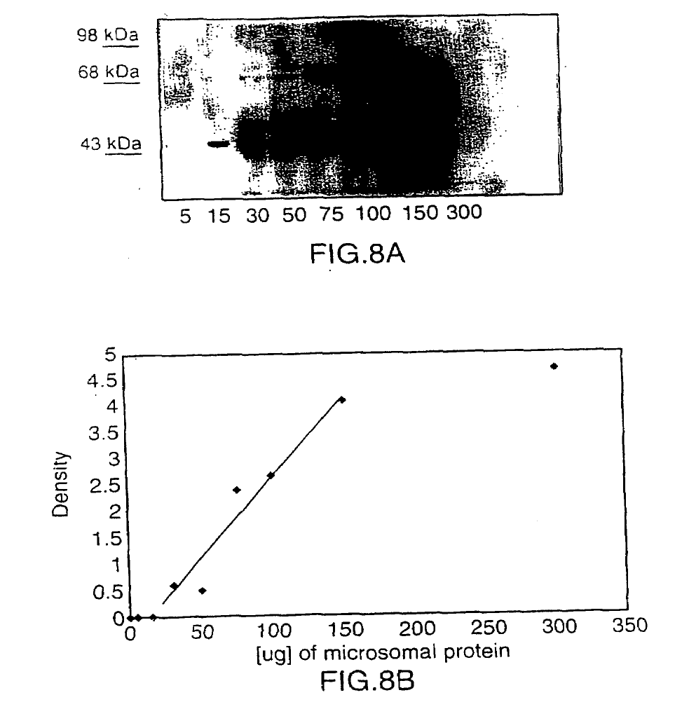

Western Blot Analysis. Liver microsomal proteins (30 μg) were resolved on 10% SDS-PAGE gels, and transferred to nitrocellulose (120 volts, 18 hrs, at room temperature) by electroblotting (Western blotting) (Guengerich et al., 1982a). Blots were blocked for 1 hr at room temperature with 2% (wt/v) BSA dissolved in 150 mM NaCl, 50 mM Tris-HCl, pH 7.4, and 0.05% Tween 20 (TBST). Incubations with primary and secondary antibodies were performed for 1 hr in TBST. The primary, and secondary antibodies consisted of the monoclonal CYP2A6 antibody (1/2000 dilution of 5 mg/ml stock; Gentest Corporation), and an anti-mouse IgG horseradish peroxidase conjugate (1/2000 dilution; Amersham Corporation, Arlington Heights, Ill.), respectively. After each incubation with primary and secondary antibodies, blots were washed three times with TBST for 10 minutes each. Blots were visualized using the chemiluminescent ECL reagent (Amersham Corporation). The densities of the visualized bands were quantified using a MCID imaging system (Imaging Co.). After determining the linearity of detection of CYP2A6 bands (FIG. 8), a concentration of 30 μg of microsomal protein was used for each liver for comparisons.

### Results:

### CYP2A6 in Nicotine Metabolism

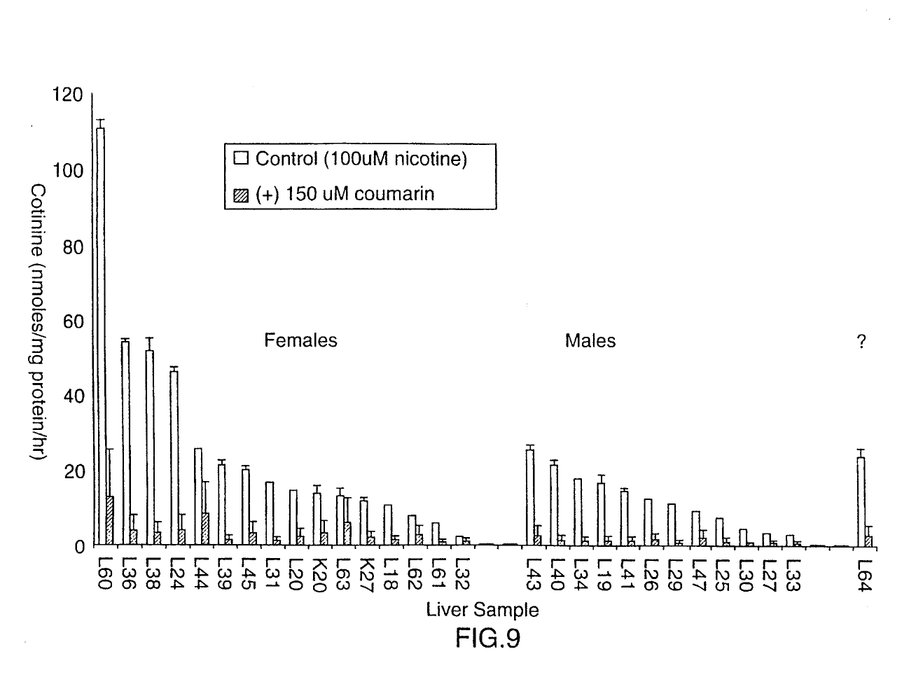

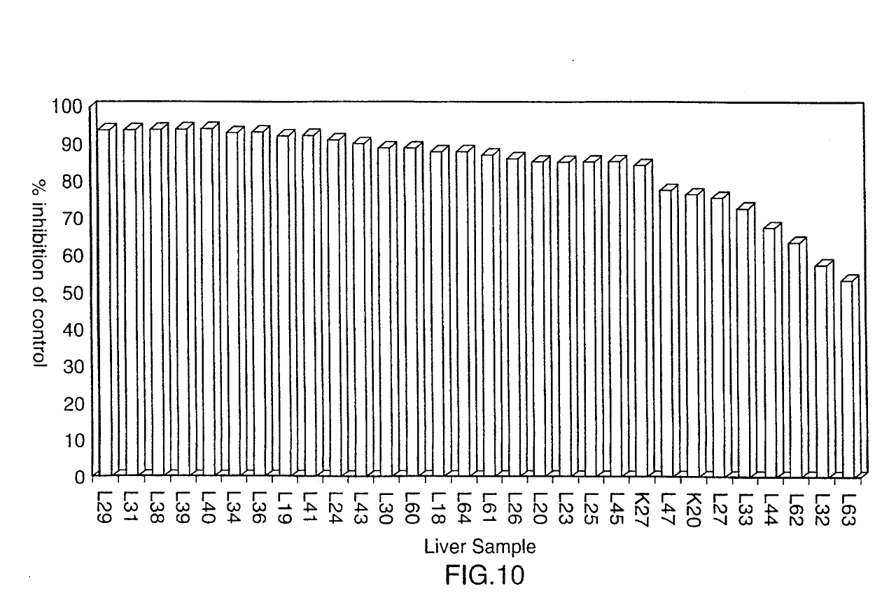

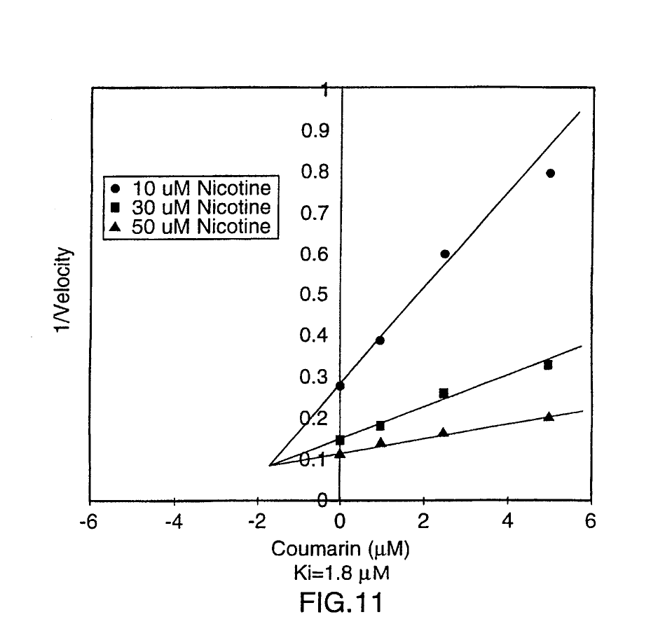

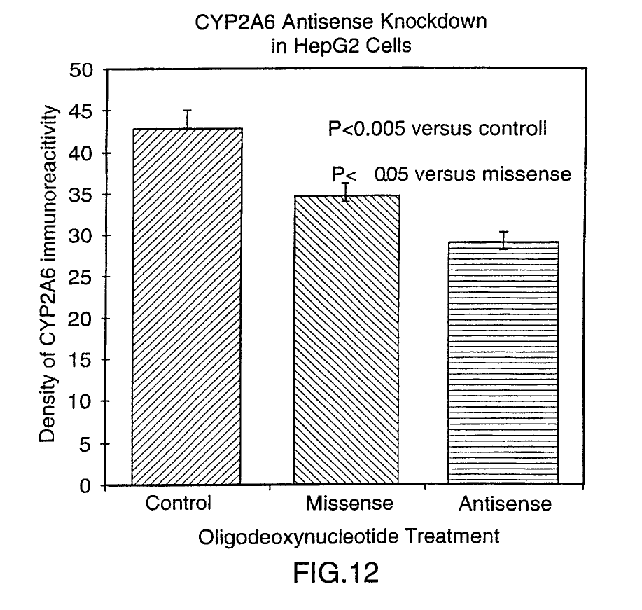

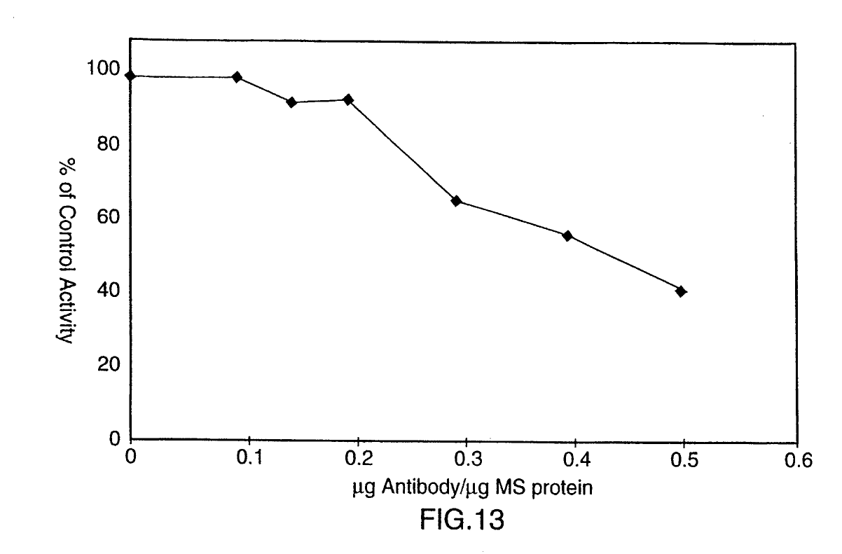

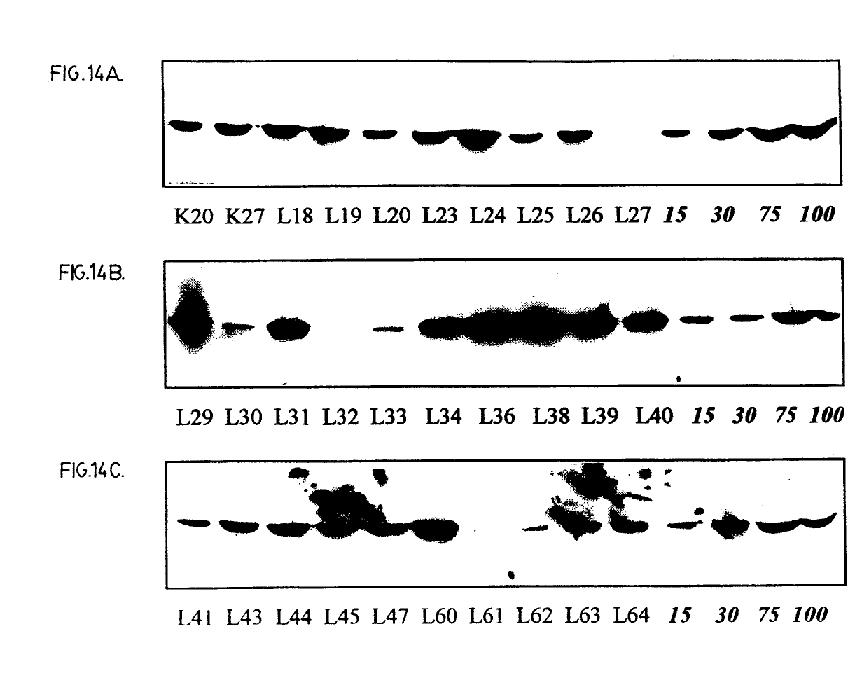

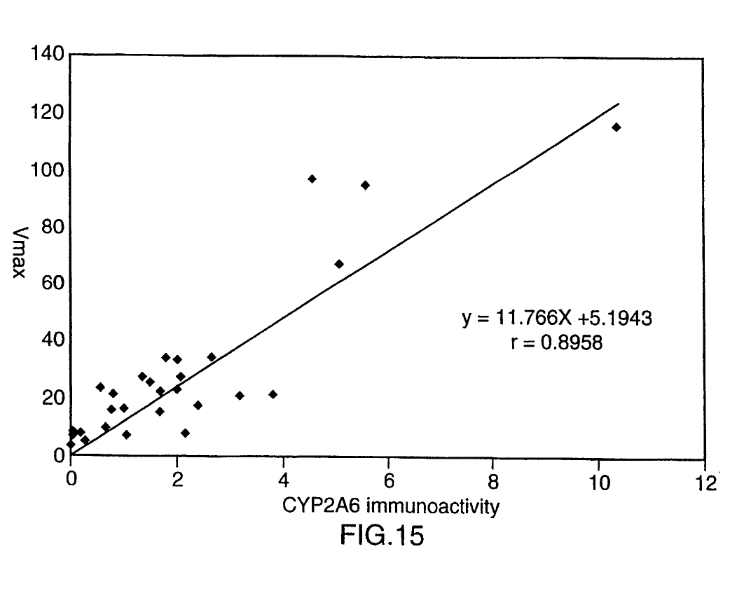

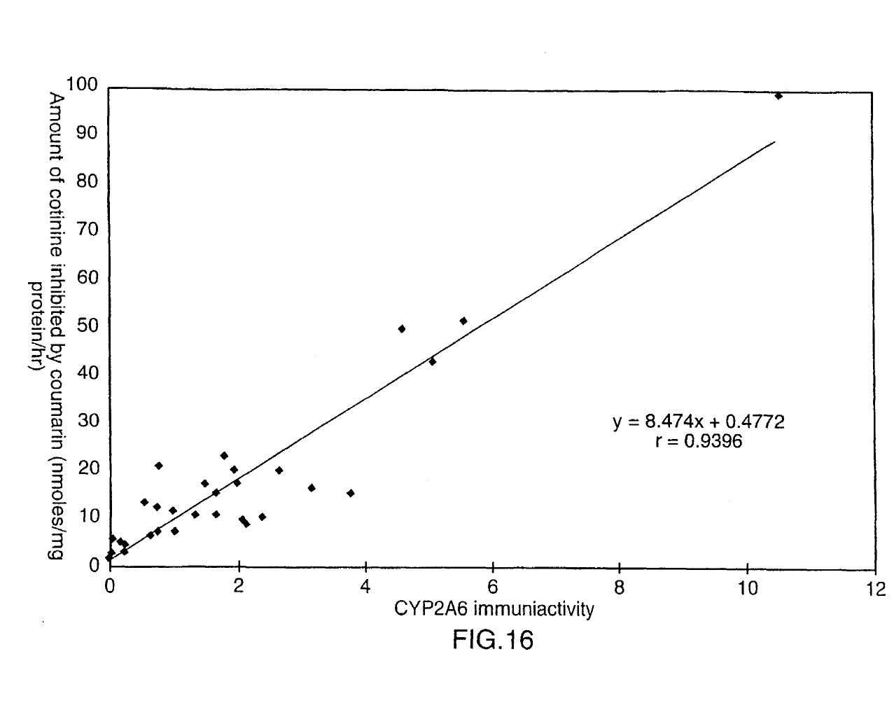

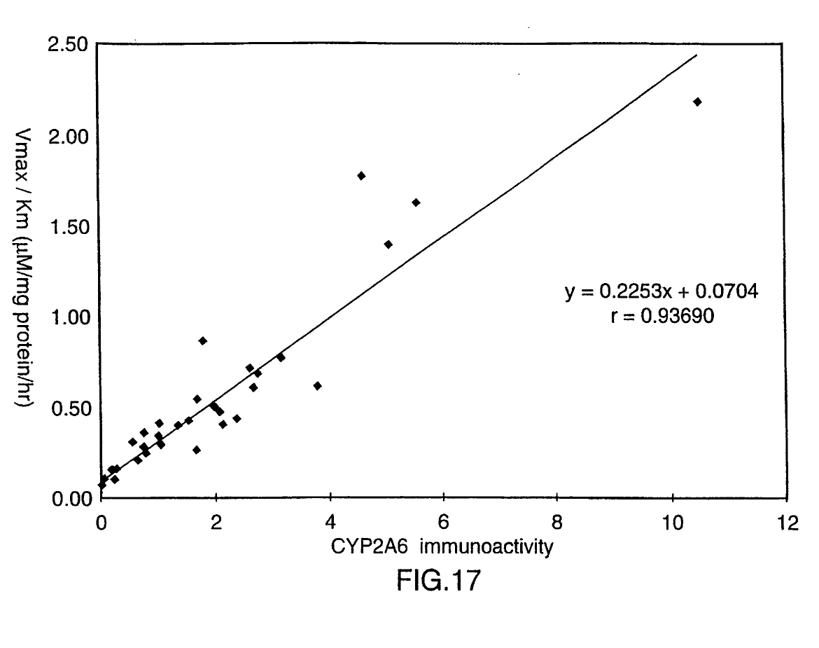

Coumarin, a specific and selective CYP2A6 substrate significantly inhibited cotinine formation with a mean inhibition of 84±11% (mean±SD) (FIGS. 9 and 10). An apparent Kᵢ value of ˜2.0 μM (n=3) was determined using K27 human liver 15 microsomes. A sample Dixon plot is shown in FIG. 11. The competitive nature of this interaction was confirmed by performing Cornish-Bowden plots (Cornish-Bowden, 1974). Coumarin, along with three analogs of coumarin, were incubated with 50 μM nicotine in K27 liver microsomes. The inhibition results and the rank order of potency for coumarin, 7-methylcoumarin, 7-methoxycoumarin, and 7-ethoxycoumarin are 20 shown in FIG. 12. Of the four compounds, coumarin had the strongest inhibitory effect on cotinine formation. Immunoinhibition experiments were carried out using a specific monoclonal antibody raised against 2A6. Results showed an over 50% inhibition of cotinine formation when 0.5 μg MAB-2A6/μg microsomes was incubated with 100 μM nicotine (FIG. 13). Immunoreactive CYP2A6 was measured in each of the 30 human liver microsomes. The densities of each band were used to compare the relative amounts of CYP2A6 between livers (FIG. 14). Band densities were standardized by dividing them by the 30 μg L64 band density of their respective blots. This was done so that individual band densities can be compared between blots. Western blot analysis was repeated at 3 and 10 μg amounts for livers that were outside the linear range, as determined by the L64 standard curves. A summary table of the values used in CYP2A6 and nicotine correlation studies is presented in Table 1. Using the band densities obtained from the Western blots, a strong correlation (r=0.90, n=30, p<0.001) was seen between CYP2A6 levels and Vₘₐₓ values of cotinine formation (FIG. 15). This r value decreases to 0.60 when the four high Vₘₐₓ livers were removed from the correlation. A stronger correlation was seen when CYP2A6 immunoactivity was plotted against the amount of cotinine inhibited in the presence of 150 μM concentrations of coumarin (r=0.94, n=30, p<0.001) (FIG. 16). This r value decreases to 0.64 when the four high Vₘₐₓ livers were removed. The Vₘₐₓ/Kₘ values shown in FIG. 17 provide an excellent measure for the efficiency of individual livers for metabolizing nicotine to cotinine (i.e. the higher the value, the more efficient the liver). These Vₘₐₓ/Kₘ values were plotted against CYP2A6 immunoactivity which resulted in a strong correlation (r=0.94, n=30, p<0.001) (FIG. 17). This correlation remained strong even when the four high Vₘₐₓ livers were removed (r=0.84).

### CYP2B6 in Nicotine Metabolism.

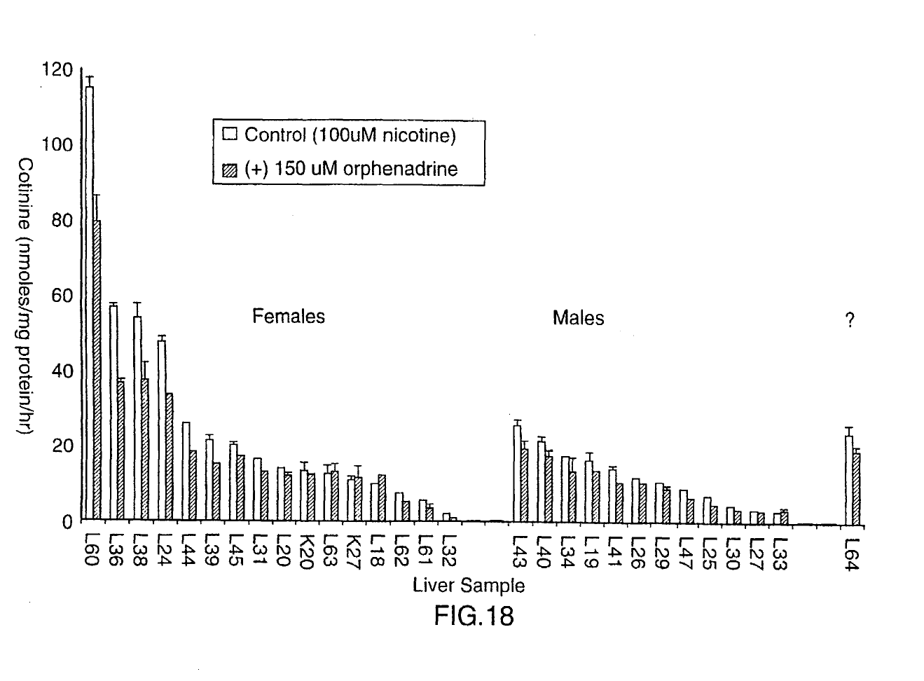

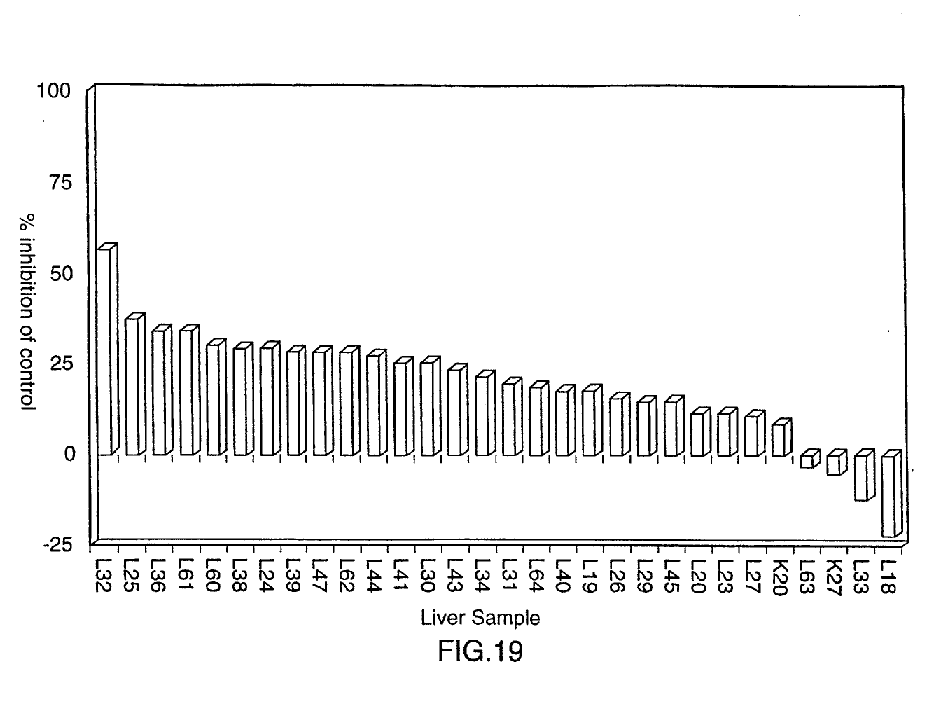

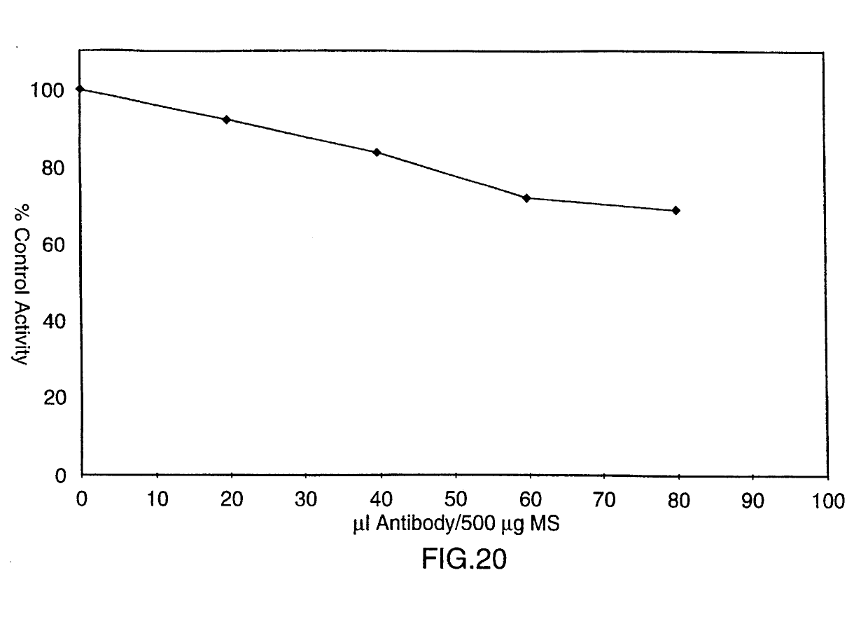

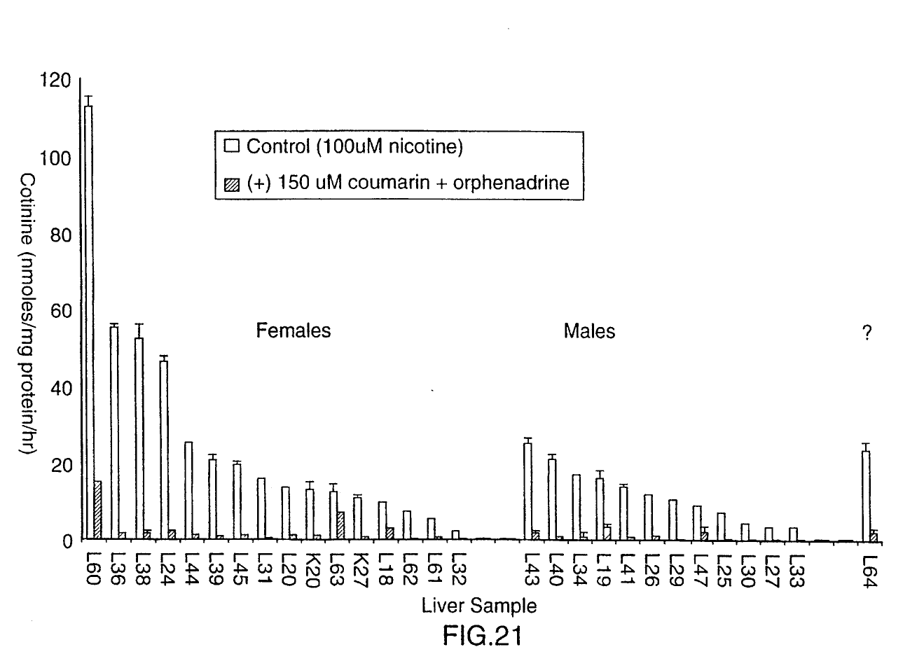

Orphenadrine, which is a CYP2B6 inhibitor, had some slight inhibition which was approximately 20±16% (mean±SD) (FIGS. 18 and 19). When antibodies raised against the rat CYP2B1 were included, a 30% inhibition of cotinine formation was seen in the K27 human liver microsomes (FIG. 20). Coumarin and orphenadrine were also used in combination and had an overall, 92±11% (mean±SD) inhibition of cotinine formation (FIG. 21).

### CYP3A4 in Nicotine Metabolism

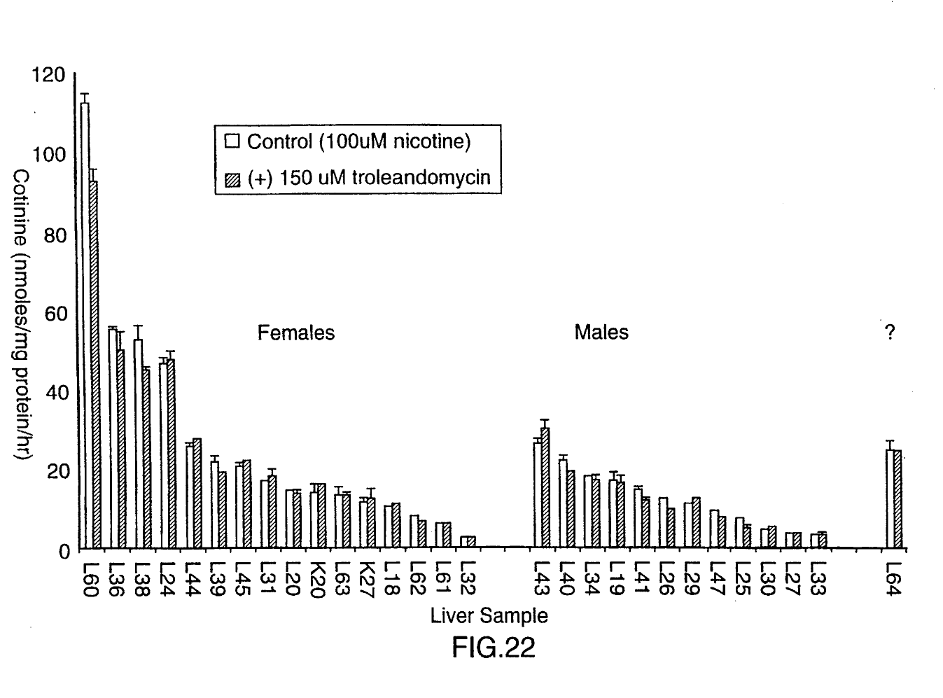

Troleandomycin, a specific CYP3A inhibitor, did not show any overall inhibition of cotinine formation. The mean inhibition was 3% of control cotinine formation, with a standard deviation of 11% (FIG. 22).

### Discussion:

Since the metabolism of nicotine to cotinine by CYP2D6 was concluded to be of minor importance the role of other cytochromes P450 was investigated. In particular, the importance of CYP2A6 and CYP2B6 was addressed since both these enzymes are variably expressed in humans, and have been shown to contain some nicotine oxidase activity (Flammang et al., 1992; McCracken et al., 1992).

In the chemical inhibition studies cotinine production was significantly inhibited after the addition of coumarin, a specific CYP2A6 substrate (Pearce et al., 1992; Yamano et al., 1990; Waxman et al., 1985). Coumarin was incubated in the presence of nicotine, across the 30 human livers. Results indicated a universal inhibition of cotinine formation. Nicotine incubations in the presence of coumarin alone inhibited cotinine formation by over 80%, and when orphenadrine was added this value increased to 91%. In particular, when coumarin and orphenadrine were used in combination, 23 of the 30 livers showed a greater than 90% inhibition of cotinine formation. In these experiments, coumarin inhibition of nicotine metabolism was found to be competitive and quite potent with a Kᵢ of ˜2.0 μM. FIG. 12 summarizes the effect of coumarin along with three analogs in inhibiting cotinine formation. The rank order of potency was coumarin >7-methoxycoumarin >7-methylcoumarin >7-ethoxycoumarin. It is interesting to note that 7-ethoxycoumarin had a lesser effect of inhibiting nicotine metabolism than coumarin, since 7-ethoxycoumarin is a well known substrate for many human cytochrome P450 enzymes (i.e., CYPs 1A1, 1A2, 2A6, 2B6, 2C8, 2C9, 2E1, and 3A4) (Waxman et al., 1991). This suggests that nicotine and coumarin metabolisms are closely related. Immunochemical inhibition studies revealed that a monoclonal antibody raised against the human CYP2A6 inhibited cotinine formation by 60%. CYP2A6 immunoactivity was also quite variable with a greater than 300 fold difference between L27 and L60 human livers. It is interesting to note that no detectable amount of this enzyme was found in the L32 liver sample, under these experimental conditions. Perhaps this individual carries variant mutant alleles for the CYP2A6 polymorphism. Western blot analysis revealed that nicotine metabolism was highly correlated to CYP2A6 levels. Nicotine to cotinine Vₘₐₓ values correlated with CYP2A6 levels (r=0.90, p<0.001) across the 30 human livers. Using the coumarin inhibition results, which can be used as a measure of relative CYP2A6 activity, an even stronger correlation was seen with immunoreactive CYP2A6 (r=0.94, p<0.001). Also, CYP2A6 levels strongly correlated with Vₘₐₓ/Kₘ values (r=0.94, n=30, p<0.001).

As exhibited in FIGS. 5 and 6, nicotine metabolism by the 30 human liver microsomes displayed large inter-individual variations. In particular, a 30 fold variation was seen between the lowest and highest metabolic rates. It is interesting to note that the four livers with exceptionally high cotinine formation were all females. In vivo studies, however, show that nicotine metabolism is more rapid in men than women (Beckett et al., 1971; Benowitz et al., 1984). There was no correlation with respect to cotinine formation and age. The differences in cotinine formation may be explained by variable expression of the CYP2A6 enzyme. The same four individuals who had exceptionally high nicotine oxidase activity also had large amounts of the CYP2A6 enzyme. One possible explanation is that the four livers showing high rates of nicotine metabolism were exposed to environmental inducers (i.e. phenobarbital) which would increase levels of CYP2A6. In summary this study indicates that CYP2A6 is very important in the human liver metabolism of nicotine.

With respect to CYP2B6, previous literature has shown that it is not constitutively expressed in human livers, and is likely induced by exposure to phenobarbital. One particular study has shown that detectable levels of this enzyme, as measured by Western blots, only occurred in 12 out of 50 livers (Mimura et al., 1993). In the present study orphenadrine and anti-rat 2B1 were used to investigate the importance of CYP2B6 in nicotine metabolism. Orphenadrine is an anti-parkinsonian agent which has been shown to form an inhibitory intermediate complex, in hepatic microsomes, only in phenobarbital induced microsomes (Reidy et al., 1989). Using 150 μM orphenadrine, in the chemical inhibition studies, resulted in an overall net inhibition of 20±16% (mean±SD). Antibodies raised against rat B1 has been shown to have affinity for the human CYP2B6 enzyme by Gentest Corp. They used similar concentrations of antibody to inhibit specific 2B6 mediated reactions. Previous cDNA work have shown that this enzyme has the highest known rate of nicotine oxidation (Flammang et al., 1992; McCracken et al., 1992). Therefore in certain individuals, who are exposed to environmental inducers such as phenobarbital, CYP2B6 may play an important role in nicotine metabolism.

Troleandomycin, a substrate for several CYP3A enzymes, was used to study CYP3As role in nicotine metabolism. It was important to answer the CYP3A question since it is the most abundant CYP found in human liver (Shimada et al., 1994). CYP3A expression is also induced by exposure to phenobarbital, hence it is potentially a great source of variability in nicotine metabolism. Thus, the use of troleandomycin in the chemical inhibition studies behaved as a negative control. The results agree with previous studies that showed no involvement of the CYP3A subfamily in nicotine metabolism.

CYP2A6 has been shown to play an important role in nicotine metabolism, thus variations in CYP2A6 expression may be directly responsible for the high interindividual variation seen in cotinine formation. Genetic variation in CYP2A6, and variable CYP2B6 expression may contribute to the 3-fold variation observed in nicotine metabolism in human subjects (Benowitz et al., 1982). Exposure to phenobarbital has been shown to have an inductive effect on nicotine metabolism by primarily increasing the expression of cytochrome P450 enzymes (Nakayama et al., 1982; Hibberd et al., 1985; Foth et al., 1990; Seaton et al., 1991; Seaton et al., 1993). Rat perfused livers, pretreated with phenobarbital, showed a 14-fold increase in nicotine elimination compared to saline treated controls (Rudell et al., 1987). Human hepatocyte studies, in which individuals were pretreated with phenobarbital, showed higher than normal nicotine oxidation rates (Williams et al., 1990). One particular study showed that the primate CYP2A mediated activity increases with exposure to phenobarbital (Pearce et al., 1992). The CYP2A and CYP2B gene subfamilies are closely linked on chromosome 19. Thus common factors may influence the expression of genes within these subfamilies in the human liver (Miles et al., 1989, 1990; Forrester et al., 1992). This implies that, along with CYP2B6, human CYP2A6 expression may be affected by exposure to phenobarbital. This, in turn, could affect the overall metabolism of nicotine from the body. Several studies have indicated that smokers adjust their smoking behaviour to try and regulate or maintain nicotine blood levels (McMorrow et al., 1983; Russel et al., 1987). Therefore rapid metabolizers of nicotine may smoke more cigarettes to maintain nicotine levels, and hence are exposed to more potentially toxic compounds. This can occur if individuals are exposed to phenobarbital, which has been shown to increase CYPs 2A6 and 2B6 mediated reactions. Conversely, slower metabolizers of nicotine may smoke less cigarettes so that toxic doses of nicotine are not achieved, or else they might be at a higher risk for nicotine-related adverse effects. This might occur in individuals who contain the variant/inactive forms of CYP2A6.

These studies have confirmed that CYP2A6 is important in nicotine metabolism, and that nicotine metabolism is quite variable among individual human liver microsomes. This variability may be a consequence of previous drug use, and in the case of CYP2A6 the presence of variant CYP2A6 alleles.

### EXAMPLE 3

### Chemical Inhibition Studies with Various Chemical Compounds

Coumarin, 7-methylcoumarin, 7-methoxycoumarin and 7-ethoxycoumarin, at 10 and 100 μM concentrations, were incubated with 50 μM nicotine in K27 human liver microsomes. Furthermore, nicotine at 10, 30 and 50 μM concentrations was incubated using K27 liver microsomes in the presence of 0, 1, 2 and 5 μM concentrations of coumarin. Other compounds, such as methoxsalen, naringenin and diethyldithiocarbamic acid were also tested for their inhibitory effect on CYP2A6.

### Materials and Methods:

Microsomes were removed from −70° C. freezer and thawed on ice. Incubation mixtures generally contained 100 μl (S)-nicotine, 100 μl NADPH (1 mM final), 200 μl human liver microsomes (0.5 mg/ml), 20 μl Wistar rat liver cytosol, 200 μl potassium phosphate buffer (pH 7.4, 40 μM final), diluted to a 1 ml final volume with 1.15% KCl. The reaction was initiated with the addition of NADPH and placing the samples at 37° C. for 45 minutes. The reaction was stopped by adding 100 μl to 20% Na₂CO₃ (pH 11.4).

Extraction: After basification with Na₂CO₃, 10 μl of ketamine (the internal standard) and 3 ml ethyl acetate was added to each sample. The samples were vortexed vigorously for 5 min., followed by centrifugation for 5 min. at 3000 rpm. The organic layer (top) was pipetted to a separate 10 ml conical tube, which contained 400 μl of 0.01 N HCl. The samples were vortexed again for 5 min. and centrifuged at 3000 rpm for 5 min. The organic layer was then discarded, and the remaining aqueous layer was dried under nitrogen at 37° C. for 30 min. to remove any remaining organic solvent. 30 μl of each sample was then subjected to High Performance Liquid Chromatography (HPLC) analysis.

HPLC: Separation of nicotine and metabolites was achieved by using a CSC-Spherisorb-Hexyl column (15×0.46 cm) and a mobile phase consisting of 20% acetonitrile and 80% 20 mM potassium phosphate, pH 4.6, containing 1 mM octanesulfonic acid was used. The separation was performed with isocratic elution at a flow rate of 1 ml/min. The retention times for cotinine, nicotine and ketamine were 3.5, 4.2, and 7.0 minutes respectively.

### Results:

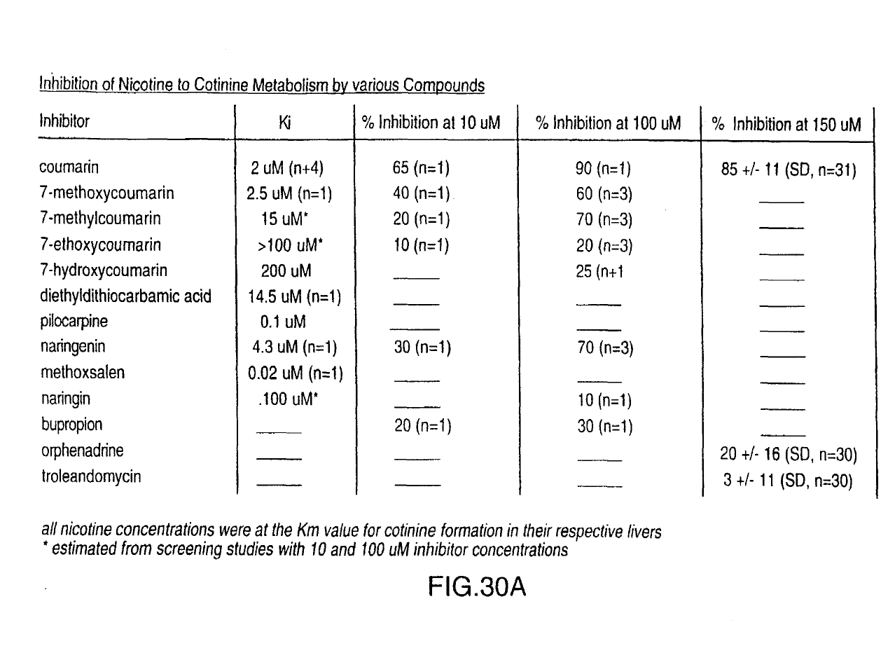

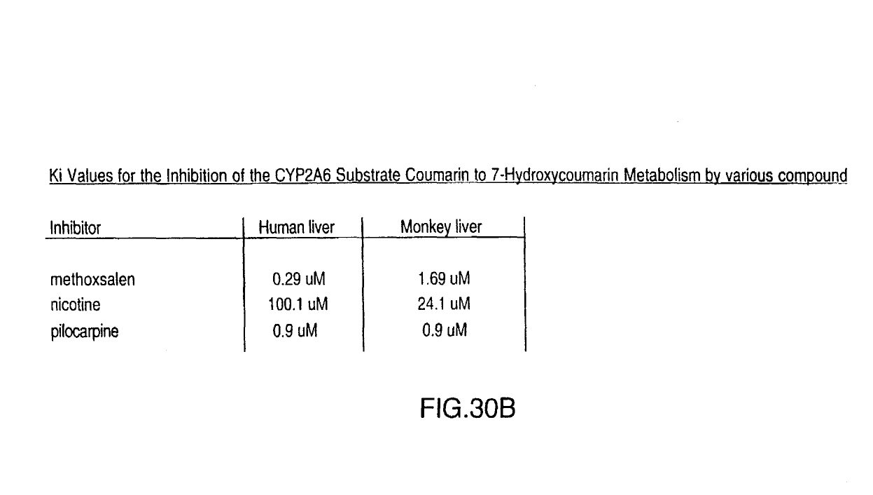

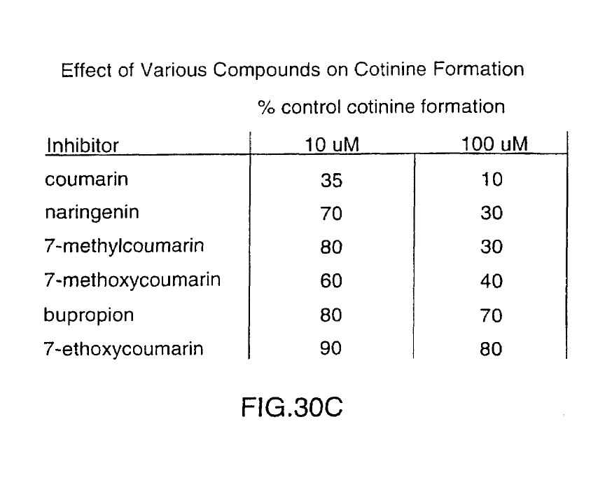

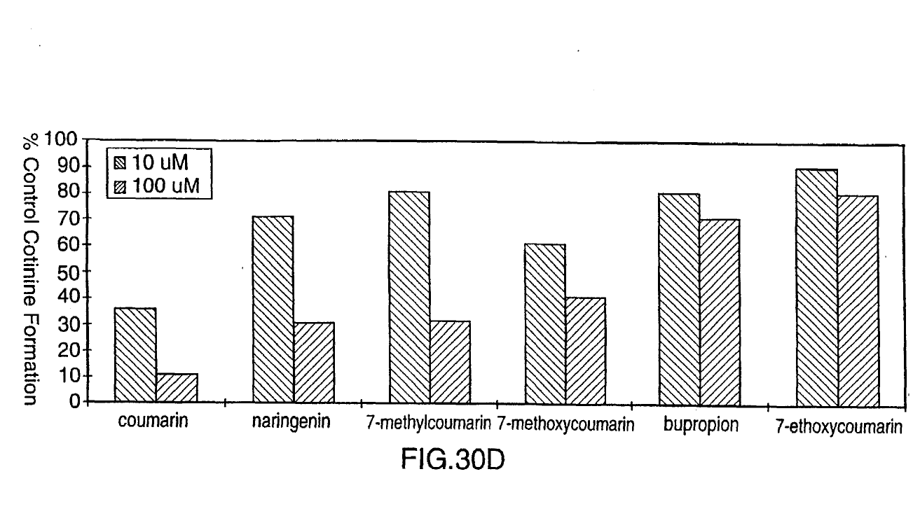

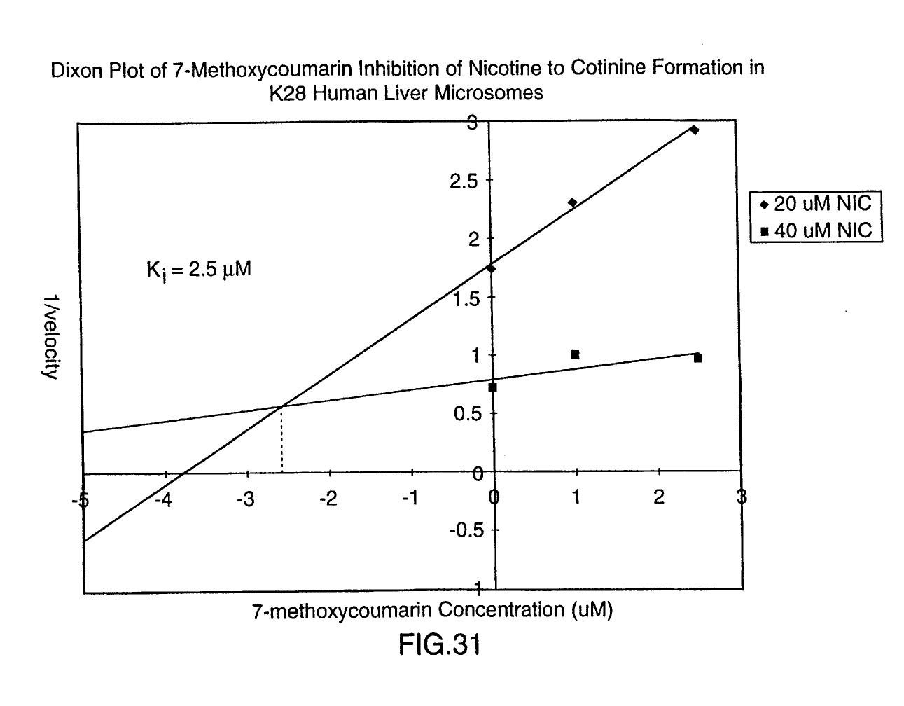

Coumarin and coumarin analogs can inhibit CYP2A6 metabolism of nicotine and several (e.g. coumarin, 7-methyl and 7-methoxy) have Kᵢ (inhibitor constants) which indicate they are potent inhibitors. See FIGS. 11, 30A, 30C, 30D and 31 in this regard. FIG. 11 shows a Dixon plot for coumarin inhibition of cotinine formation where nicotine at 10, 30 and 50 μM concentrations was incubated using K27 liver microsomes in the presence of 0, 1, 2.5 and 5 μM concentrations of coumarin. FIG. 31 shows a Dixon plot of 7-methoxycoumarin inhibition of nicotine to cotinine formation in K28 human liver microsomes.

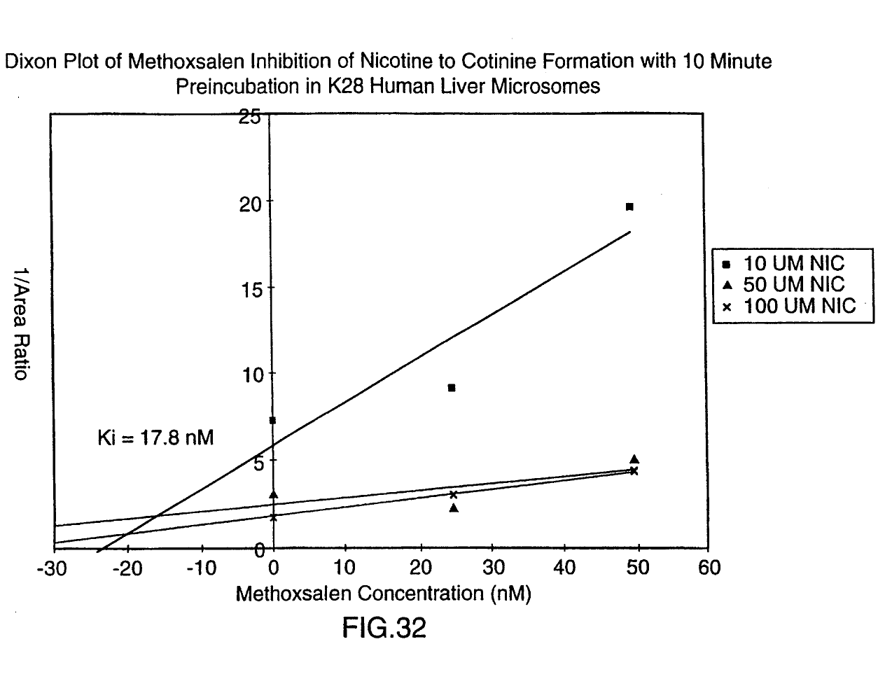

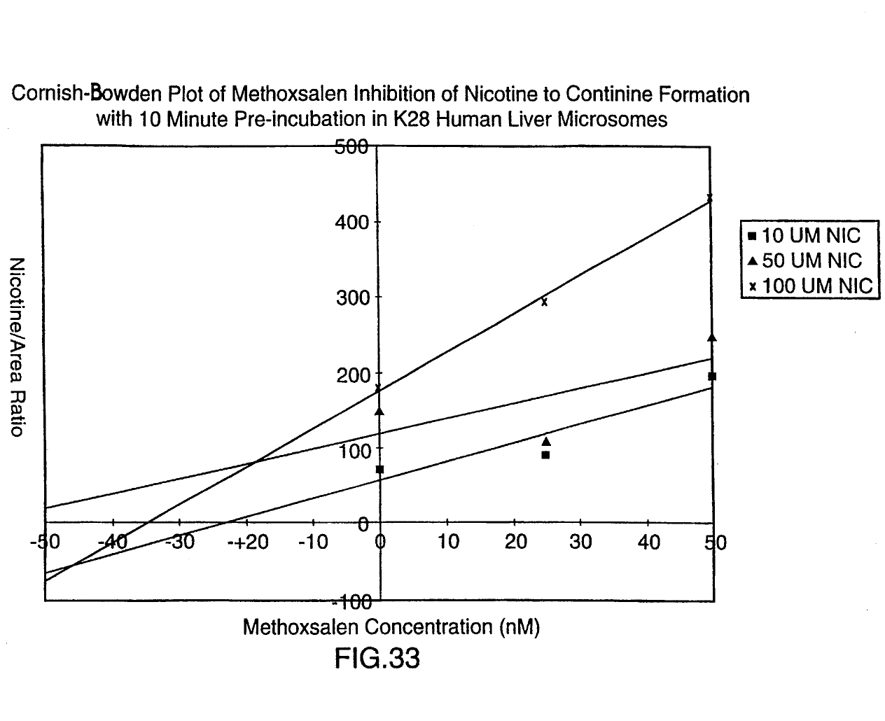

The data as displayed in a Dixon plot shows that methoxsalen is an extremely potent inhibitor of CYP2A6 metabolism of nicotine (Ki=20 nM) (See FIG. 32). From the Cornish Bowden plot in FIG. 33, the mechanism of inhibition appears to be mixed.

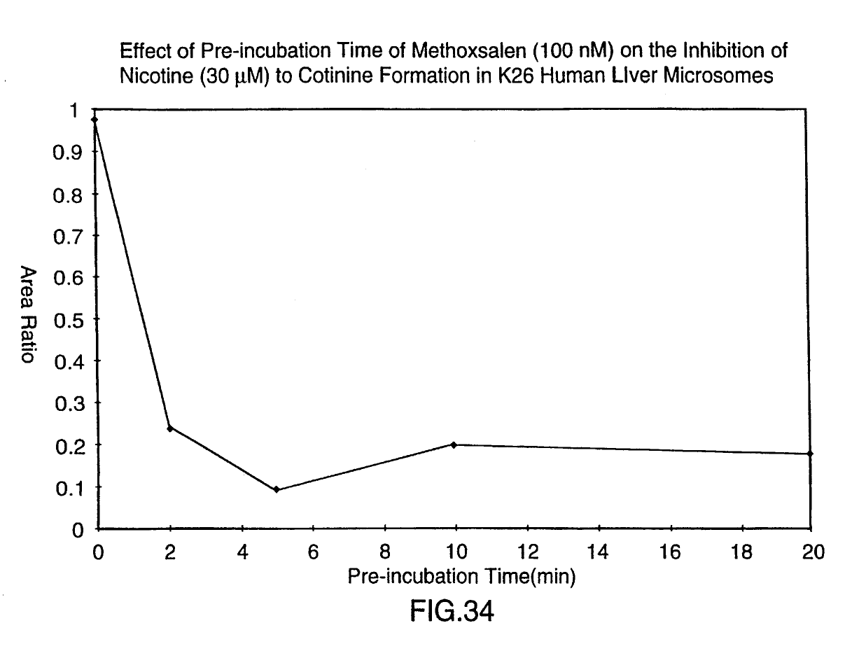

The data also demonstrates that methoxsalen inhibition of CYP2A6 increases with pre-incubation of methoxsalen with human liver microsomes (see FIG. 34). This suggests that the mechanism of inhibition is not purely competitive and may be mechanism-based, or irreversible. Clinically, inhibition of CYP2A6 by methoxsalen might be expected to outlast the presence of the drug in the plasma.

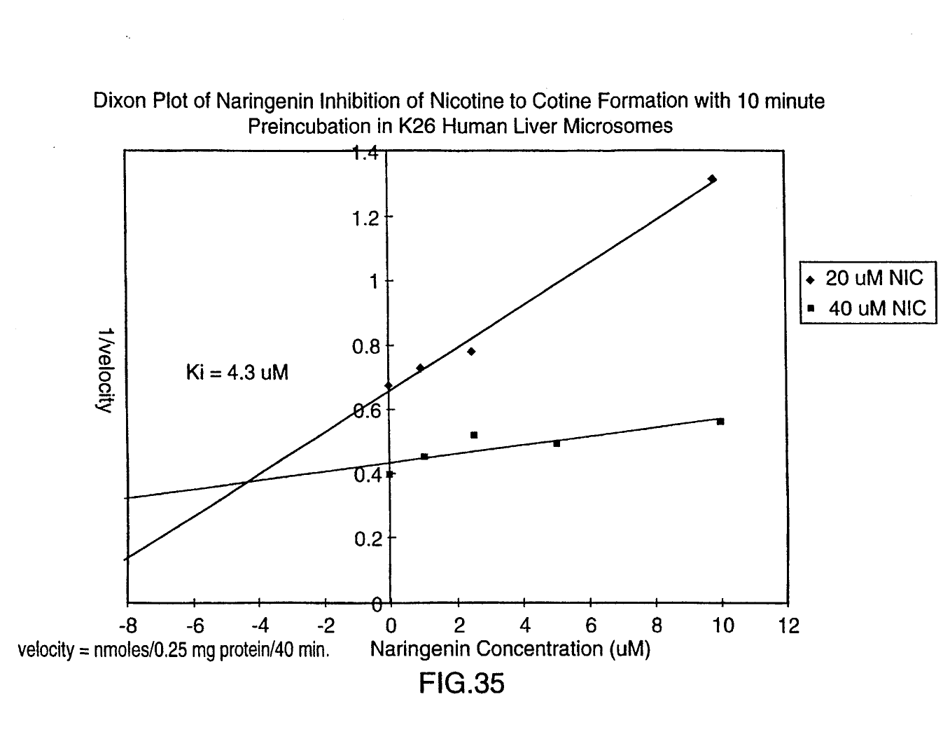

As shown by the Dixon plot in FIG. 35, a naturally occurring flavone found in plants and fruits such as naringenin potently inhibits CYP2A6 metabolism of nicotine (Ki=4.3 μM). The mechanism of this inhibition may be irreversible and the duration of CYP2A6 inhibition longer than the presence of the substance in the blood.

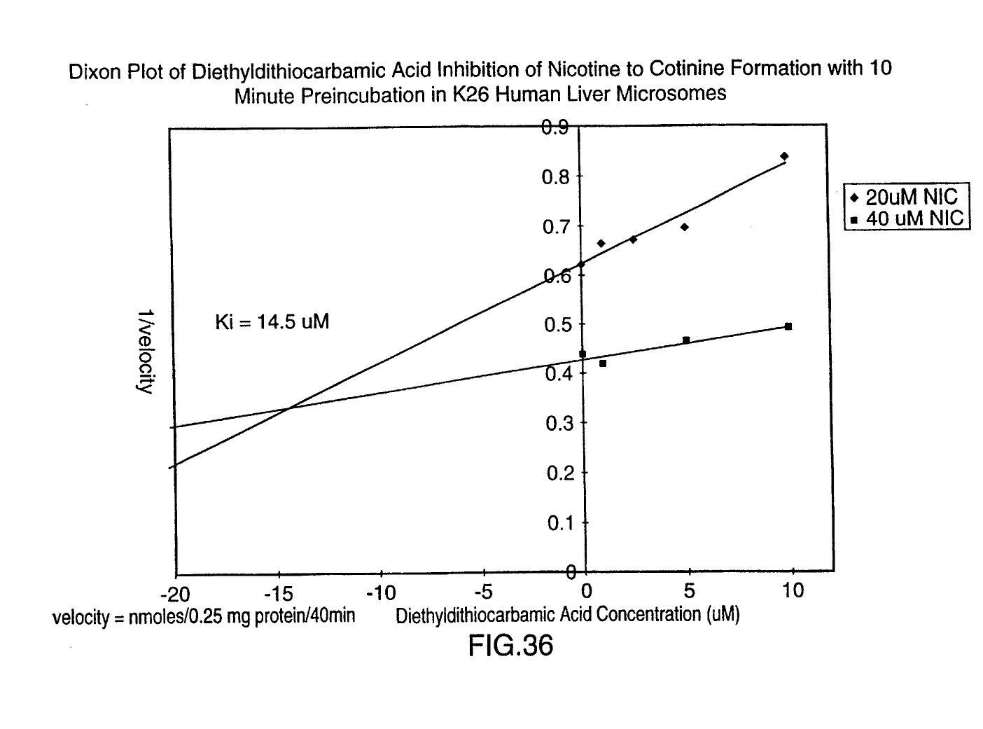

Finally, the data summarized in the Dixon plot in FIG. 36 shows that diethyldithiocarbamic acid inhibits nicotine metabolism by human liver microsomes with high affinity (Kᵢ=14.5 μM). Diethyldithiocarbamic acid is a metabolite of a marketed drug disulfiram. This suggests this drug may be useful in treating tobacco dependence.

### EXAMPLE 4

### Clinical Studies

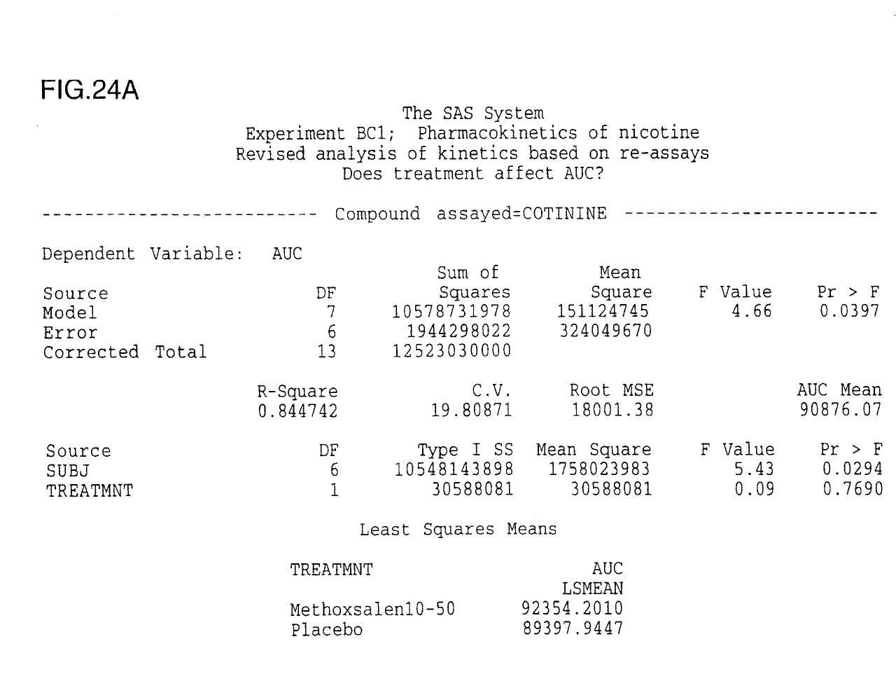

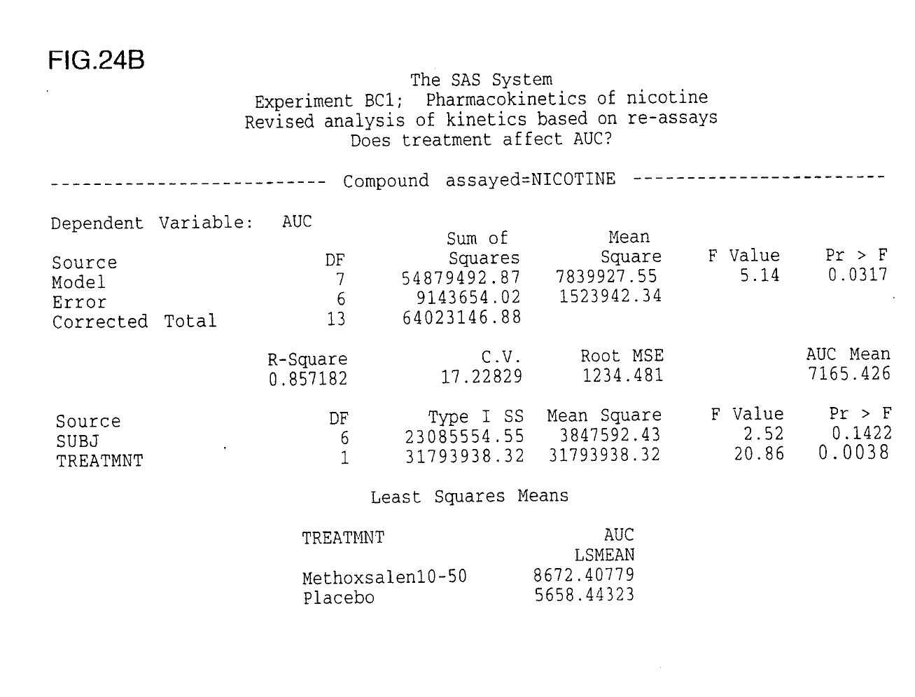

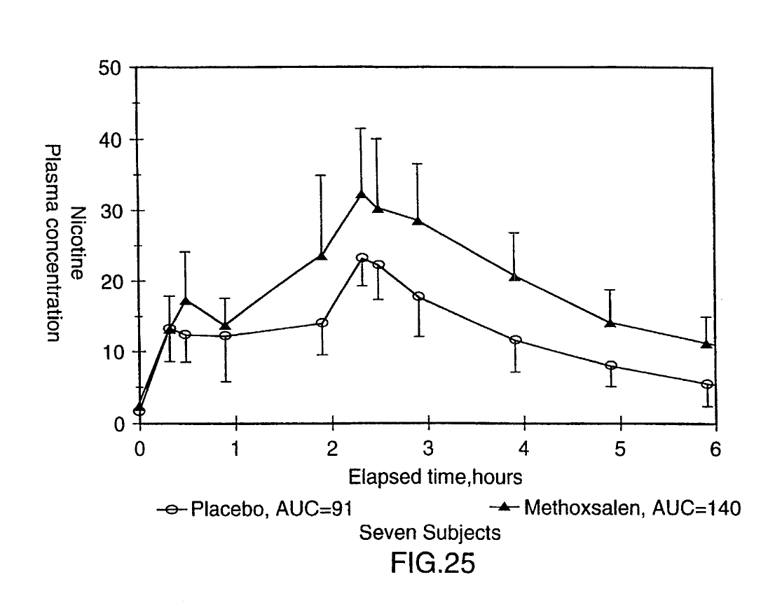

Initial clinical studies have been conducted. These have demonstrated that inhibition of CYP2A6 results in a highly significant and large mean 54% increase in the area under the plasma nicotine concentration curve in seven (7) dependent smokers administered subcutaneous nicotine (31 μg/kg) compared to placebo pre-treatment (see FIGS. 24 and 25).

### Materials and Methods:

### Design Overview:

In this study, dependent smokers were abstinent for 8 hours and then received either placebo or methoxsalen (20 to 40 mg based on weight) orally 30 minutes before the first of three subcutaneous nicotine injections given at 0, +1 and +2 hours). Methoxsalen is a potent inhibitor of CYP2A6 (see FIG. 32) with a half-life of about 1 hour in humans. Frequent blood samples were drawn over 6 hours for measurement of nicotine and cotinine concentrations in plasma and measures of nicotine effects (e.g., heart rate; blood pressure; symptoms and urges and desires to smoke).

### Study Day Schedule:

Each subject abstained from tobacco, food, beverages (other than water), and any inconsistently used drugs from midnight before each study day but continued to take any regularly scheduled drugs allowed by the protocol (e.g., oral contraceptives, daily vitamins). Before baseline measures were taken, a breath CO sample (Ecolyzer) was taken to assess compliance with the smoking abstinence (<10 ppm expected). The subsequent daily schedule is summarized here. All measurements are with respect to a time zero at 8 AM, at which time the first of three hourly nicotine injections were given. Nicotine was injected at 0, +1, and +2 hours.

Baseline physiologic and subjective measures were taken at −30 minutes, +30 minutes, and hourly thereafter until +5.5 hours, approximately coinciding with the expected peak plasma nicotine concentrations after each injection. Blood samples were taken at least hourly, more often around the expected peaks after the first and third injections, as described below.

A standard breakfast (but without caffeine) was served after the first blood sample and injection, and a standard lunch (but without caffeine) was served during the day. Subjects were medically assessed for discharge at the end of the test when all measures were complete. Subjects were not allowed to smoke until after their discharge.

Methoxsalen/placebo capsules were administered 30 minutes prior to the first nicotine injection.

There was a washout period of 1 or 2 days between consecutive test days.

### Drug Treatments:

Placebo and methoxsalen capsules were used. The following table shows the doses of methoxsalen that were administered.

The manufacturers recommended dosage schedule was used in this study.

| DOSE | WEIGHT (kg) |
|---|---|
| 10 | <30 |
| 20 | 31-50 |
| 30 | 51-65 |
| 40 | 66-80 |
| 50 | >80 |

Sterile nicotine bitartrate was obtained from Sigma Chemical. The reported purity is >99.5%. Nicotine bitartrate injections of 31 μg/kg (expressed as the base) in sterile saline were used. Each of these three daily injections delivered 2 mg to a 70 kg subject, a mass of drug comparable to that delivered by one cigarette. The solutions were passed through a viral filter to remove any remote risk of viral or bacterial contamination.

Using an indwelling venous catheter, 8 ml blood samples were drawn every hour from +0 to +6 hours, and additional blood samples were taken at 15, 25, and 40 minutes after the first and third nicotine injections to characterize the kinetics around the expected time of the peak concentration, 25 minutes from injection. Samples that could not be immediately centrifuged for extraction of plasma were stored on ice for at most one hour before centrifugation.

Three separate urine samples were collected—a baseline voiding and two four-hour pooled samples.

### Assays:

Plasma (nicotine, cotinine) and urinary nicotine and cotinine were measured using an HPLC ion exchange column assay, using an electrochemical detector for nicotine and a UV detector for other compounds. The sensitivity of the nicotine assay was <1 ng/ml and that of the cotinine was <5 ng/ml. Conjugates in urine were determined after hydrolysis with beta-glucuronidase.

Creatinine was measured, allowing all drug concentrations to be re-expressed as a ratio of the other substance to creatinine. This provided some control for variability in urine dilution or the duration or efficiency of urine collection.

### Results:

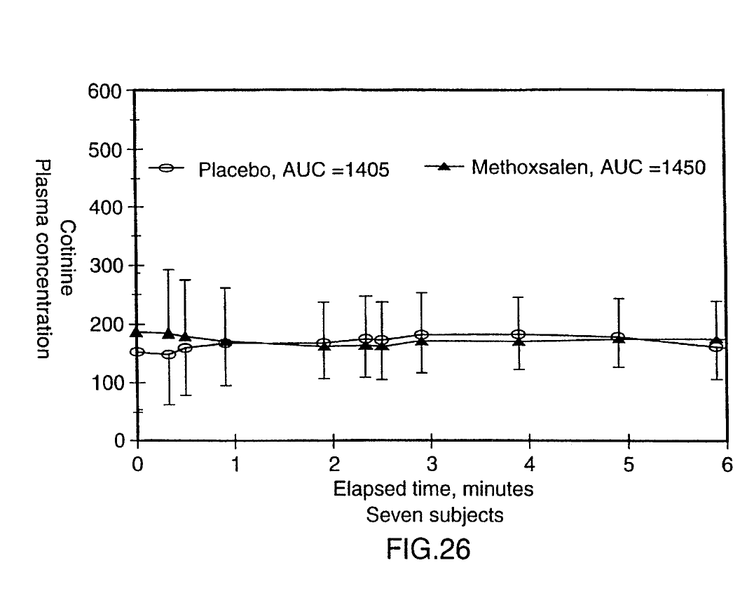

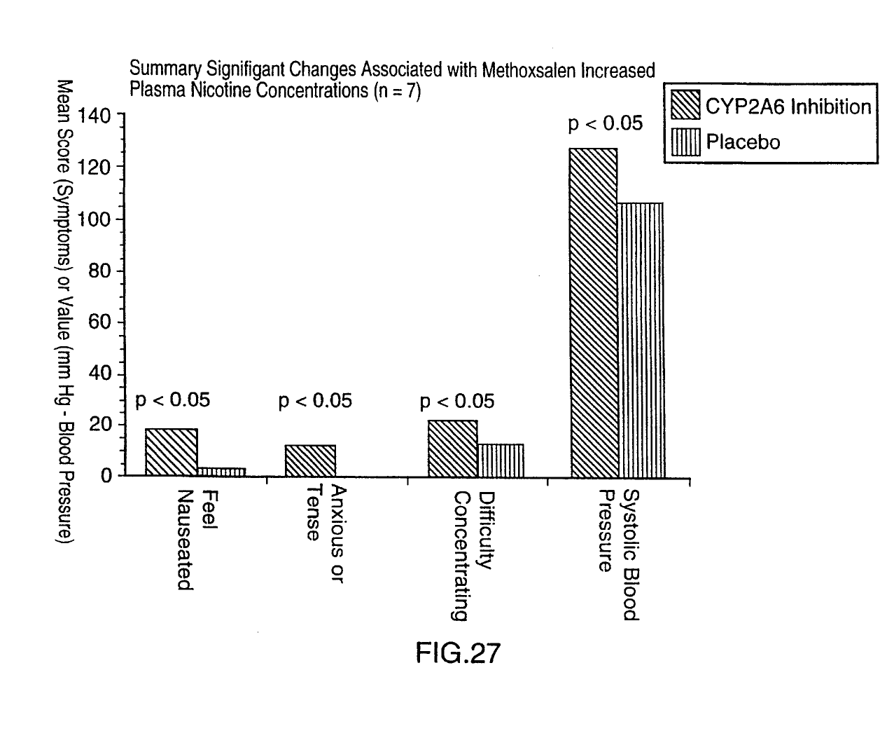

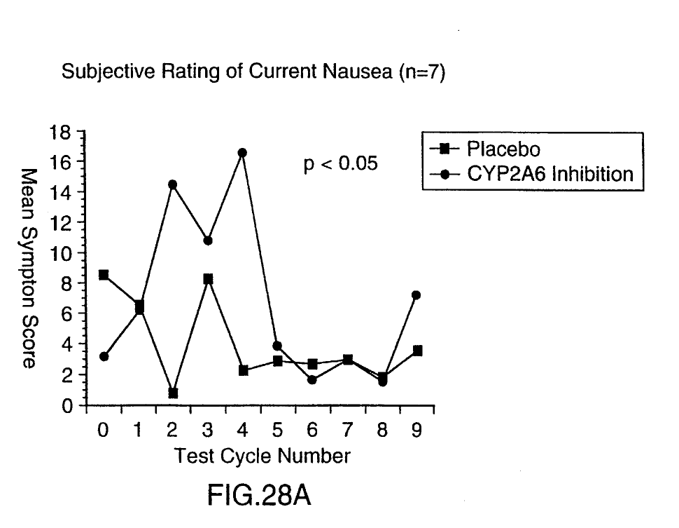

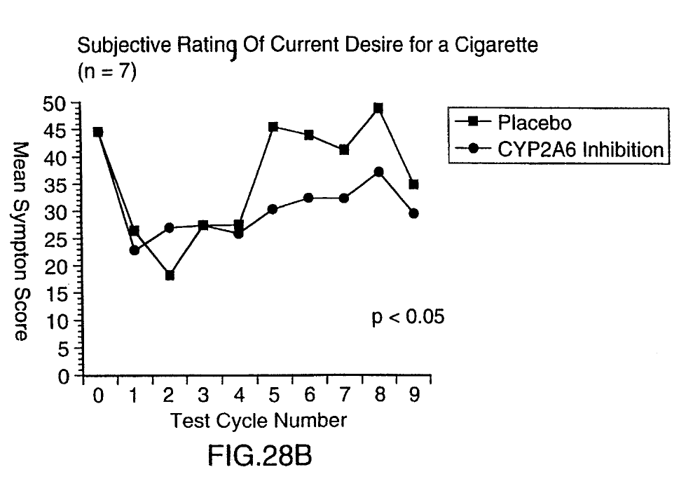

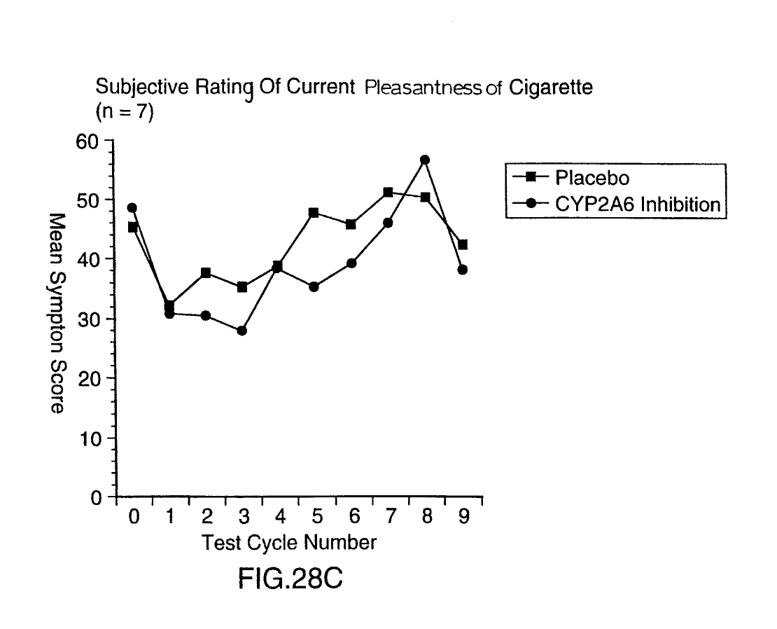

FIG. 25 shows the large changes caused by methoxsalen in nicotine concentrations. FIG. 26 shows the effect over time of methoxsalen on the cotinine plasma concentrations of seven subjects. FIG. 27 and FIGS. 28A to 28C indicate that these increases in plasma nicotine were accompanied by a significant increase in nausea, anxiousness, difficulty concentrating, systolic blood pressure and a significant decreased desire to smoke and urge to smoke and a significant decrease in the expectation that a cigarette would be pleasant. These findings clearly indicate that smokers felt less need to smoke when treated with the CYP2A6 inhibitor.

### EXAMPLE 5

### Demonstration that Antibodies Against CYP2A6 Can Block Nicotine Metabolism

### Immunoinhibition Experiments

Immunoinhibition experiments consisted of incubating 0.5 mg/ml K12 liver microsomes with CYP2A6 (monoclonal), CYP2B1 (polyclonal), CYP2 μl (polyclonal), CYP2D6-peptide (polyclonal), and CYP3A2 (polyclonal) antibodies. BSA, rabbit and goat antisera were used as negative controls. Antibodies and microsomes were preincubated on ice for 30 min followed by the addition of 100 μM nicotine, 1 mM NADPH, and 20 μl rat cytosol in 0.04 M phosphate buffer (pH 7.4). Subsequent incubations were for 45 min at 37° C.

### Results:

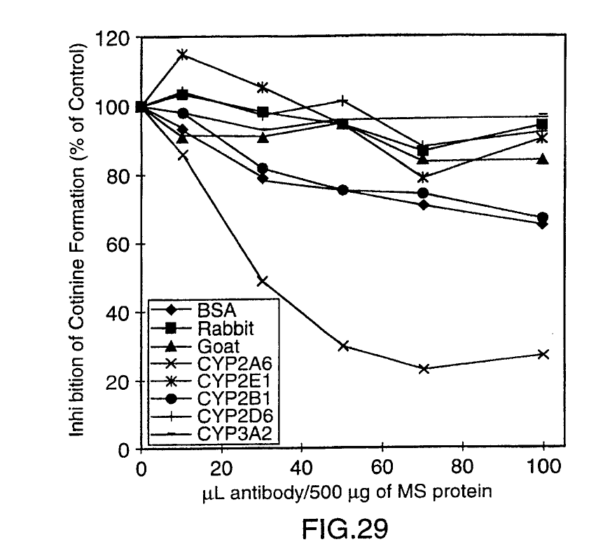

The CYP2A6 antibody selectively inhibits cotinine formation and produces >80% decrease in nicotine metabolism by CYP2A6 (see FIG. 29).

### EXAMPLE 6

### Antisense Study

In order to test the feasiblility of using antisense oligodeoxynucleotides (ASO) for the reduction of the CYP2A6 enzyme we designed 4 ASO to four different parts of the human CYP2A6 gene and mRNA sequence. The ability to decrease CYP2A6 protein was tested in 2 different human cell lines. The first was a human lymphoblast cell line (h2A6) with a plasmid expression system containing the human CYP2A6 cDNA which is commercially available from Gentest Corp (Woburn, Mass.) and the second was a human hepatic cell line which expresses CYP2A6 (HepG2).

The h2A6 P450 cells were grown to 3×10⁶ cells, the media removed and 5 μg ASO and 20 μg/ml lipofectin was added in 1 ml of 5% horse serum supplemented media. The cells were grown for 24 hr when a further 4 mls of complete media (10% horse serum) was added and the cells were grown for an additional 48 hr. Each cell sample was then washed 3 times with phosphate buffered saline, pelleted and frozen. Western blots were carried out on the samples and it was determined that only the ASO #23 (e.g., 3′ prime end of exon 2) was effective at removing the CYP2A6 immunoreactivity. These studies were then repeated using the addition of missense oligodeoxynucleotides (MSO) controls for this sequence. The MSO controls have 2 nucleotides switched at the 5 and 3 prime end. Again, we found that the ASO#23 effectively deceased the amount of CYP2A6, relative to untreated control cells and MSO#23-treated cells.

The human hepatic HepG2 cells, which express CYP2A6, were grown and aliquoted at 1.0×10⁵ cells and grown for 48 hrs in 2 mls complete media (10% fetal calf serum). After 48 hr, 2 μg ASO oligos and 15 μg/ml lipofectin was added in 1 ml 5% FCS media and grown for 24 hr. Cells then trypsinized and washed for Western blots or fixed with 4% paraformaldehyde for Immunocytochemistry. Again we found that only ASO#23 was able to decrease the CYP2A6 protein. FIG. 12 illustrates the profound decreases in CYP2A6 protein after treatment with ASO#23, relative to control untreated and MSO#23 treated cells.

These data suggest that CYP2A6 can be dramatically decreased using molecular techniques, in this case using antisense technology, and further supports the proposed application of these techniques to treating nicotine dependence.

### Antisense Oligonucleotide (ASO) Sequences Used in CYP2A6 Knockdown Experiments

Sequence data for antisense oligodeoxynucleotides

| | | | | | | | | | | | | | | | | | | | | | | | | |
|---|---|---|---|---|---|---|---|---|---|---|---|---|---|---|---|---|---|---|---|---|---|---|---|---|
| NAME* | START** | 1 | 2 | 3 | 4 | 5 | 6 | 7 | 8 | 9 | 10 | 11 | 12 | 13 | 14 | 15 | 16 | 17 | 18 | 19 | 20 | 21 | 22 | END |

| | | | | | | | | | | | | | | | | | | | | | | | | |
|---|---|---|---|---|---|---|---|---|---|---|---|---|---|---|---|---|---|---|---|---|---|---|---|---|
| ASO#15 | −25 | T | A | G | A | G | G | G | A | T | G | A | T | A | G | A | T | G | G | T | G | A | C | −4 |
| ASO#13 | 171 | C | T | T | C | A | T | G | A | G | G | G | A | G | T | T | G | T | A | C | | | | 189 |
| ASO#25 | 190 | G | G | C | C | A | T | A | G | C | G | C | T | C | A | C | T | G | A | T | | | | 208 |
| ASO#23 | 333 | C | C | A | T | A | G | C | C | T | T | T | G | A | A | G | A | C | C | C | A | G | | 353 |
| MSO#23 | 333 | C | C | C | C | A | G | C | C | T | T | T | G | A | A | G | A | C | A | T | A | G | | 353 |

*The sequences are named according to the exon that they are designed for (e.g., ASO#23 is found in exon 2 at the 3 prime end)
**The start and end nucleotide numbering is based on the CYP2A6 mRNA sequence found in the Genbank database (HUMCPIIA3A, accession numbers M33318 and M33316) and in Yomano et al., Biochemistry 29(5); 1322-1329, 1990.

### EXAMPLE 7

### Epidimiology Study

We examined the prevalence of CYP2A6 gene mutations in 126 tobacco dependent Caucasian smokers and 143 Caucasian individuals who had tried smoking, but who had never became tobacco dependent smokers (e.g., exposure controls). The objectives were two fold. The first was to determine the incidence of individuals who were deficient in CYP2A6 activity (e.g., homozygous for null CYP2A6 alleles). The second was to determine if slower CYP2A6 mediated nicotine metabolism, due to having null CYP2A6 alleles, decreased the chances of becoming a tobacco dependent smoker.

### Materials and Methods:

### Primers Used for PCR Genotyping Assays:

!Assay? Name? Sequence (5′-3′)

| | | |
|---|---|---|
| CYP2A6*2 (v,) | F4 | CCTCCCTTGCTGGCTGTGTCCCAAGCTTAGGC |
| and | R4 | CGCCCCTTCCTTTCCGCCATCCTGCCCCCAG |
| CYP2A6*3 (v₂) | E3F | GCGTGGTATTCAGCAACGGG |
| | E3R | TCGTGGGTGTTTTCCTTC |

### CYP2A6 Genotype

DNA is extracted from blood samples and quantified using routine extraction procedures. CYP2A6 genotype was determined using nested PCR and RFLP as described by Fernandez-Salguero et al (1995). The first amplification, which is CYP2A6 genespecific, was used to increase the specificity for the CYP2A6 gene (versus other CYP2A genes). Exon 3 was utilized in the second amplification because both the CYP2A6*2 and CYP2A6*3 mutant alleles contain nucleotide changes leading to amino acid changes in this region of the CYP2A6 gene.

The first amplification was performed using the XL-PCR kit (Parkin-Elmer Co., Norwalk, Conn.). A 100 μl reaction mixture of 0.2 μM of primer F4 and R4, 200 μM dNTPs, 0.8 mM magnesium acetate, and 2 U of rTthl DNA polymerase and 400 to 600 ng of genomic DNA used. The amplification was performed in a MJ DNA Engine (MJ Research, Inc., Watertown, Mass.) at 93° C. for 1 minute, 66° C. for 6 minutes and 30 seconds for 31 cycles.

The second amplification was performed in a reaction mixture containing 0.5 μM of primers E3F and E3R, 200 μM dNTPs, 1.5 mM MgCl₂, 2.5 U of Taq DNA polymerase (Gibco BRL, Life Technologies, Burlington, Ontario), and 2.5 μl of first amplification product, which was the template for the reaction. The reaction conditions were as follows: 94° C. for 3 minutes, followed by 31 cycles of 94° C. for 1 minute, 60° C. for 1 minute and 72° C. for 1 minute.

The second amplification yielded a PCR product 201 bp in length which was digested with Xcm I (New England Biolabs) and Dde I (New England Biolabs and Pharmacia Biotech) to detect the CYP2A6*2 and CYP2A6*3 mutations, respectively (cutting indicates the presence of the mutation). Concentrations of enzymes and PCR product, total volume and digestion time were determined empirically to optimize cutting efficiency with a minimal amount of time and enzyme. Xcm I digestion reactions were carried out at 37° C. for 2 hours in a 30 μl reaction mixture containing 1× NEBuffer 3 (100 mM NaCl, 50 mM Tris-HCl, 10 mM MgCl₂, 1 mM DTT pH 7.9 @ 25° C.), dH₂O, and 2 U of Xcm I. Dde I digestions were carried out at 37° C. for 2 hours in a 30 μl reaction mixture containing One-Phor-All (OPA) buffer (Pharmacia Biotech) and 2 U of Dde I. Digestion products were analysed on ethidium-stained 3% agarose gels.

Blood samples were obtained under consent from all subjects (n=269). In addition, detailed smoking histories and tobacco/nicotine dependence (DSM RIII and IV criteria) were obtained through structured interviews. Less than 1 percent of the smokers were homozygous for CYP2A6 null alleles (deficient metabolizers). This demonstrates that for almost all (>99 percent) smokers, inhibiting the CYP2A6 enzyme will result in altered nicotine kinetics with less than 1 percent being unaffected. It indicates that over 99% of smokers are candidates for this novel tobacco dependence therapy involving CYP2A6 inhibition.

We examined whether there were fewer slow CYP2A6-mediated nicotine metabolizers (e.g., homozygous or heterozygous for null CYP2A6 alleles) in the smokers when compared to a control group who had tried smoking but never become dependent.

### Results:

We found twice as many homozygous null individuals (completely CYP2A6 deficient) individuals in the control group versus the smokers. In addition, there were nearly twice as many slow (heterozygous for CYP2A6 null alleles) CYP2A6 nicotine metabolizers in the control group (19%) relative to the smokers (12%). This suggests that some protection against nicotine dependence is imparted by carrying the null alleles for CYP2A6.

### EXAMPLE 8

### Studies of the Coumarin Metabolic Pattern

A study has been conducted to determine (i) if coumarin metabolic pattern (recovery and ratios of free, 7-hydroxy and conjugated 7-hydroxy coumarin) reflects CYP2A6 genotype, independent of current smoking status, and nicotine (NIC) metabolism determined from smokers' blood and urine samples; (ii) if acute smoking affects coumarin metabolism in smokers; and (iii) if there are differences between male and females in the prediction of NIC metabolism from coumarin metabolism.

### Materials and Methods:

Equal numbers of medication free, healthy males and females of any racial background currently tobacco dependent (n=10 of each sex) and non-smoking (n=10 of each sex) were genotyped (CYP2A6) and had a Coumarin Test on 2 separate days, once between 7:00 and 9:00 a.m. and once between 2:00 and 4:00 p.m. Smokers were required to have the morning test after abstaining from smoking for at least 8 h (for example 8a.m. before the first cigarette) and the afternoon test on a normal smoking day. Urine and plasma samples, which can be analyzed for NIC and cotinine (COT) (free and conjugated), were taken prior to each Coumarin Test, along with breath carbon monoxide to determine smoke exposure. Coumarin and free and total 7-hydroxycoumarin were analyzed by HPLC using U.V. detection with 4-OH coumarin as an internal standard as modified after Rautio et al. (1992).

### HPLC Analysis of 7-Hydroxycoumarin in Urine and Plasma:

### (1) Sample Preparation:

Urine or plasma samples (0.5 ml) are hydrolyzed with 0.25 ml of β-glucuronidase acetate buffer solution (15 mg/ml acetate buffer, 0.2 M, pH 5.0) at 37° C. for 30 min. Extraction is followed with 2 ml ether by vertex for 5 min and centrifuged at 3000 rpm for 10 min. Ether extract (1.2 ml) is transferred to another clean tube and dried under nitrogen gas. The residue is reconstituted in the HPLC mobile phase (see below), and injected onto HPLC.

### (2) HPLC Analysis:

The HPLC system consists of Hewlett Packard 1050 HPLC system (pump, autosampler and UV detector) and HP339611 integrator. The chromatographic separation was performed with an HP Spherisorb-ODS2 column (125×4 mm I.D., 5 μm). Samples were eluted with a mobile phase of acetonitrile:water:acetic acid of 150:850:2 (v/v/v) at a flow rate of 1.0 ml/min, and monitored by a UV detector at a wavelength of 324 nm n. Samples are quantitatively determined by an external standard method.

### Results:

Blank urine or plasma samples showed no interfering peak for 7-hydroxycoumarin. Sensitivity of this method is 1 ng/ml urine or plasma. Intraday and inter-day variations are less than 10%. This analysis is linear from 10 ng to 4000 ng/ml.

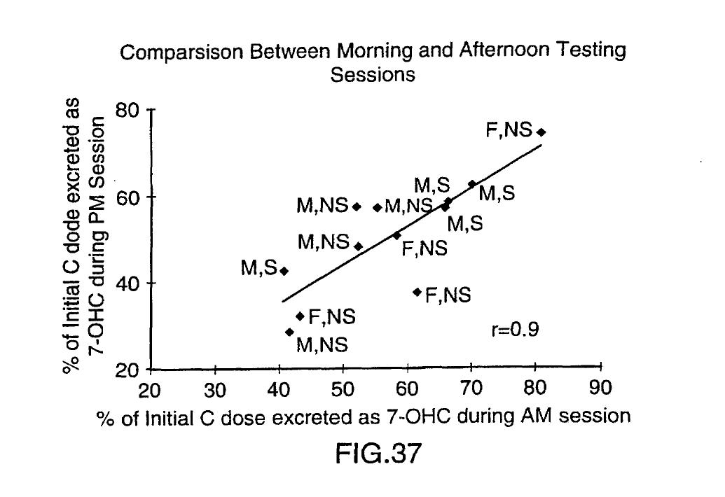

FIG. 37 illustrates a correlation between fasted morning and non-fasted afternoon coumarin (C) testing sessions. The values in FIG. 37 are expressed as the percentage of an initial 5 mg coumarin dose excreted as total 7-hydroxycoumarin (7-OHC) (r=0.9, p<0.001). The labels on FIG. 37 represent male smokers (M,S), male nonsmokers (M, NS), female smokers (F, S), female nonsmokers (F, NS). In the specific experiment that generated the results illustrated in FIG. 37, subjects were given 5 mg of coumarin p.o., and urine was collected for the next 4 hours. Testing occurred during 2 fasted morning sessions and 2 non-fasted afternoon sessions to determine the interday and intraday variation in total 7-hydroxycoumarin excretion. The values in FIG. 37 are expressed as the percent of an initial 5 mg coumarin dose excreted as 7-hydroxycoumarin within 4 hours. Morning and afternoon values were matched per subject such that the first morning value and the first afternoon value were plotted as one point and the second morning and second afternoon value were plotted as a second point per subject.

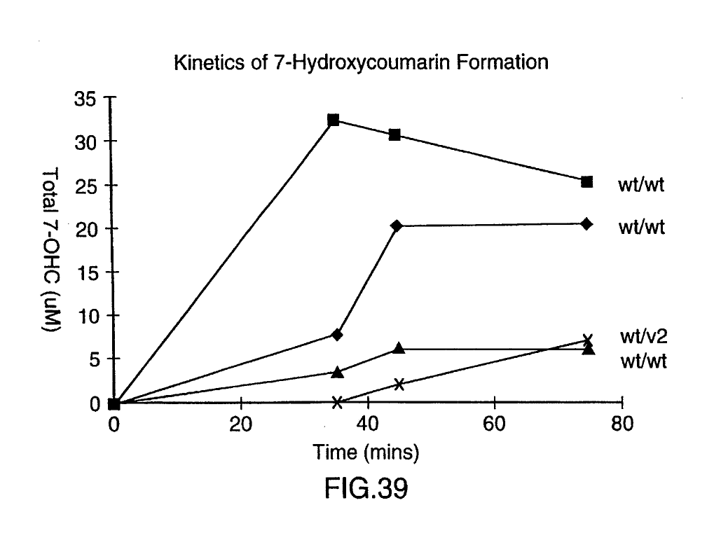

Furthermore, FIG. 39 is a graph showing a time course of total 7-hydroxycoumarin concentration detected in the plasma of subjects given 100 mg of coumarin. FIG. 39 illustrates various time courses based on corresponding genotypes for CYP2A6.

### EXAMPLE 9

### C-2 Acute Effects of CYP2A6 Inhibition on NIC Disposition and Smoking

Benowitz has used a 30-minute deuterium-labelled NIC-d2 infusion (with COT-d4) to study the kinetics and fractional clearance of NIC in smokers and non-smokers. The kinetics of the deuterated NIC are very similar to unlabelled NIC. The advantage of the non-radioactive label is that the NIC-d2 and resulting COT-d2 can be used as quantitative measures of NIC metabolism in smokers while smoking. This approach will be used to obtain a quantitative estimate of NIC to COT conversion by giving a dose of NIC-d2 sufficient to detect in the urine, taking advantage of the fact that NIC and COT are found in much higher concentrations in urine than in plasma. In preliminary studies smokers infused with 2 μg/kg/min×30 minutes were found to have urine concentrations of NIC and COT that were >80 and >600 ng/ml, respectively. Therefore it is estimated that as little as 0.2 to 0.8 mg of NIC-d2 will result in quantifiable NIC-d2/COT-d2 in the urine. This would correspond to a 6 minute infusion of the 0.5 μg and 2.0 μg/kg/30 min infusions of Benowitz in non-smokers and smokers. Ratios of NIC-d2/COT-d2 in urine collected over 8 h, “Nicotine-d2 Test”, will provide a direct estimate of NIC to COT conversion. This procedure will be piloted to establish dose, dose rate and analytic sensitivity. Plasma and urinary NIC, COT and trans-3′-hydroxycotinine and their glucuronides will be measured using a existing GC assay as modified in Dr. Jacob's lab (1988 protocol). Conjugates will be determined after alkaline hydrolysis (NIC and COT) or hydrolysis with β-glucuronidase (3′-hydroxycotinine). The quantisation limit is 1 ng/ml NIC and 10 mg/ml COT, with detection 50% lower. Coefficients of variation range from 1.1 to 7.8% for NIC (1-100 ng/ml). NIC-d2, COT-d2 will be determined by GC-MS (111).

The results of the experiments described in Example 2 indicate that CYP2A6 is the primary contributor to NIC disposition, with CYP2B6 important in a few individuals (=16%). Coumarin is a potent inhibitor of NIC metabolism to COT ex vivo in human liver microsomes (Ki=1.5 μM), and orphenadrine is a potent inhibitor of human CYP2B6 (Ki=3.8 μM), with an estimated half-life of about 14 h. Most smokers' NIC metabolism can be inhibited by coumarin, the remainder by an appropriate combination of coumarin and orphenadrine, and such inhibition will reduce smoking behaviour.

A study has been designed to determine the extent to which combinations of inhibitors of CYP2A6 and CYP2B6 will modify NIC metabolism in vivo and smoking behaviour in a social controlled setting. CYP2A6 genotyped current tobacco dependent (DSM-IV) individuals (n=6 wt/wt, n=6 wt/mut) will have CYP2A6 activity assessed on six occasions by a “Coumarin Test” along with measurement of plasma NIC and COT. After familiarization with the inviting social setting study site, subjects will abstain from smoking until they arrive on each study day, when they will provide a baseline urine (NIC/COT), a plasma sample (NIC/COT), and a breath CO. They will receive, on separate days, all combinations of 3 coumarin conditions (placebo, 50 mg b.i.d. or 100 mg b.i.d.) with 2 orphenadrine conditions (placebo or 200 mg); the two combinations of two active drugs will occur on days 5 and 6, after tolerance to the individual components has been verified on days 1-4 in a random, counterbalanced order. A great deal of work has been done to establish the relationship of number of cigarettes, NIC intake, smoke exposure and NIC and COT plasma (blood) concentrations and urinary excretion. The best correlations are obtained between blood NIC (4:00 p.m., 0.79), CO Hbg (0.67), urinary COT 24 h (0.62), blood COT (4:00 p.m., 0.53). COT, because of its longer half-life is less critically affected by sampling time and can be used to estimate daily NIC intake. 30 minutes after study drug, a tracer dose of Nicotine-d2 Test 1 will be given. Subjects will then collect their urine for 3 h (“Coumarin Test”) and 4 h separately for determination of NIC-d23/COT-d2 ratio (7 h) and coumarin and total and free 7-OH coumarin. Additional blood samples will be collected 0.5 (coumarin/7-OH-coumarin), 3 and 7 h (NIC/COT) after the pulse tracer dose commences. Breath CO will be determined at the same times. During this period subjects will be permitted to smoke their usual brand ad lib, drink caffeinated beverages, play games, watch videos, etc. The number of cigarettes used and residual butt weight recorded.

### EXAMPLE 10

### Smoking Reduction by Inhibition of CYP2A6

Inhibition of metabolism of NIC to COT will allow smokers to maintain plasma NIC with less smoke exposure and reduce the secondary reinforcement of smoking behaviour as a component of eventual smoking cessation. Because NIC is the addictive agent in tobacco dependence and smokers regulate their brain NIC within a fairly narrow individual concentration band, selective inhibition of NIC conversion to COT should result in a decrease in smoke exposure (i.e., “smoking”). Some individuals may require different combinations of CYP2A6 and CYP2B6 inhibitors to achieve sufficient modification of NIC metabolism.

A preliminary study has been designed to confirm the efficacy and safety of CYP2A6 inhibitors and the need for CYP2B6 inhibition in the reduction of smoking exposure and as an aid in smoking cessation.

Male or female DSM-IV dependent tobacco users who want to stop smoking, who do not want NIC substitution treatment, have made at least three serious unsuccessful attempts to stop, have no medical contraindications to participation, and have CYP2A6 wt/wt or wt/mut genotype will be eligible to participate. Prior to the trial, susceptibility to CYP2A6 activity inhibition will be assessed by stable-labelled “Nicotine Test” after coumarin 50 or 100 mg b.i.d. (Human Study C-1), to assess the change in the individual urinary NIC-d2/COT-d2 ratio. Based on the results of the study each subject will be assigned to a CYP2A6 inhibition “responders” or “poor responders” group. Therefore the study will be a comparison of placebo (n=30) vs. coumarin (n=30) in CYP2A6 high inhibition responders and of coumarin (n=7) vs. coumarin+orphenadrine (n=8) in CYP2A6 poor responders for 2 weeks. The coumarin doses may be 100 mg b.i.d. and the orphenadrine 100 mg p.o. daily.

Tobacco smoking behaviour will be monitored by daily smoking diary cards, twice weekly home carbon monoxide breath sample collected in remote CO exposure collection bags (mid-afternoon between 2:00 and 6:00 p.m.), twice weekly salivary COT. Subjects will be given instructions with respect to the purpose of treatment, plus limited supportive counseling, plus structured self-help advice. Patients will be seen weekly between 2:00 and 6:00 p.m. each day at which time plasma NIC/COT, breath CO will be done.

The primary dependent variables are measures of smoke exposure (diaries and CO measurements), averaged over the 2-week study; such a mean is sensitive to both downward and leftward shifts of the consumption-over-time curve. These variables will be analyzed as a function of treatment within high and poor responding groups separately. For two-group comparisons, n=30 per group has approximately 97% power to detect a difference of 1 S.D. between their means.

### EXAMPLE 11

### CYP2A6 Inhibition in Smoking Reduction and Cessation

Based upon studies such as outlined in Example 4, a double-blind trial to confirm the efficacy of CYP2A6 inhibition on smoking reduction and cessation will be carried out. The drug choice and doses will be determined by the studies as outlined in Example 4. A positive treatment control (e.g., NIC patch) will be tested.

Patients identical to those participating in the previous study (Human Study C-3) will enter a double-blind placebo controlled randomized trial of smoking reduction and cessation to be achieved and maintained over 12 weeks comparing coumarin and placebo (n=60) per group. The assessment and procedures will be similar to the study as outlined in Example 4. However, those actually receiving the CYP2A6 inhibitor will receive active drug for a 2-week period followed by a 2-week placebo period. This 4 week on/off cycle will be repeated 3 times with a goal of cessation at the end of 12 weeks. CYP2A6 inhibition should decrease smoke exposure by decreasing the number of cigarettes or by altering smoking behaviour. Subjects will, at the end of each “active” drug/placebo 2-week phase, be told to maintain their lower smoking behaviour for 2 weeks. This 2-week period is one of behavioural change. The inhibitor-placebo cycle will be repeated. Subjects will be seen weekly at which time their self-report smoking logs will be reviewed along with their progress (minimal adjunctive care). After the trial, subjects will be contacted at 3, 6 and 12 months and provide at least a salivary COT to determine maintenance of quit rates. Breath CO, salivary, plasma NIC/COT will be determined. Established criteria for cessation will be used.

### EXAMPLE 12

### Chronic Coumarin Effects on Smoking Behaviour

Hypothesis: That chronic coumarin administration will decrease smoking behaviour in a three day out patient paradigm.

Rationale: The effects of coumarin may not be apparent in a short term setting, particularly if smokers needs to learn to adjust their consumption when internal cues (e.g., nicotine levels) and external cues (e.g., time from last cigarette) conflict. This project will test the effect of chronic coumarin administration on smoking behaviour and nicotine levels over a three day period. The subjects will tested once with placebo and once with coumarin, in a randomized blinded design. The impact on number of cigarettes smoked, nicotine levels and resultant behavioural indices (somnolence, alertness, nausea) will be determined in normal living conditions (e.g., normal 3 days of activities).

Design: Smokers with varying degrees of cigarette consumption (>30, <30) will receive a dosage of coumarin (using a dose of coumarin which effectively alters nicotine levels: derived from Project 1) or placebo for three consecutive days (both conditions tested in all individuals). Smokers from both genotypes will be included, allowing us to assess safety (e.g., nausea) and effectiveness (only in wild type or works for both groups?). Cigarette consumption and behavioural indices as well as nicotine, cotinine and CO measures will be determined daily. Effectiveness of coumarin in decreasing cigarette consumption, increasing nicotine, decreasing CO (cigarette exposure) will be determined. In addition the relative effect on heavy or light smokers, and first, second and third days, will be determined.

### EXAMPLE 13

### Acute Effects of Coumarin on Smoking Behaviour in a Social Setting

Hypothesis: That acute administration of coumarin (inhibiting nicotine metabolism), resulting in increased nicotine, will decrease smoking behaviour in an acute social setting.

Rationale: This product may be more or less effective in an acute versus chronic dosing regime. It would also be beneficial to have a product which could be tailored to acute high risk occasions (bar nights, parties, occasions with other smokers, locations with strong smoking associations).

Design: Smokers with varying degrees of normal cigarette consumption will be dosed acutely (1 day) with coumarin or placebo, prior to an acute high risk “party” and alcohol event. Smoking behaviour, nicotine, cotinine, coumarin and CO levels, as well as behavioural indices will be determined. These studies will be done in individuals of both genetic groups (see Example 7).

### EXAMPLE 14

### In Vivo Testing of Chemical Inhibitors

Chemicals found to be inhibitors in in vitro screening can be efficiently and effectively screened for their therapeutic potential in human subjects.

### (i) Nicotine Test of Inhibited Metabolism —HPLC Analytical Technique

Nicotine bitartrate 31 μg/kg (expressed as the base) is administered subcutaneously in the absence and the presence of pretreatment with a single dose or multiple doses of the inhibitor to be tested after at least an 8 hour smoking abstinent period and blood samples are collected at 0, 20, 30 and 60 minutes after the injection of nicotine. The concentration of nicotine and cotinine in the plasma is determined by a highly sensitive HPLC method.

Determination of Nicotine Cotinine in Plasma by HPLC:
(a) Extraction procedure: Pipet into each tube (12 ml) 1 ml of sample, 50 μl of the internal standard (N-ethylnornicotine), and 1 ml of trichloroacetic acid (10%). Cap the tubes tightly, vortex-mix for a few seconds and centrifuge them at 30,000 g for 5 min. Decant the clear supernatant into a second set of clean tubes. To this protein-free plasma extract, add 0.5 ml of a 5M potassium hydroxide solution and 6 ml of methylene chloride. Cap the tubes, agitate for 30 min in a horizontal shaker and centrifuge to separate the phases. Aspirate the aqueous (top) layer and add 3.00 ml of 0.5 N hydrochloric acid solution to the organic phase and vortex-mix for 30s. Separate the phases by centrifugation and remove and discard the organic (lower) layer. To the acidic aqueous solution remaining in the tube, add 0.5 ml of 5M potassium hydroxide solution and 5 ml of methylene chloride and vortex-mix for 30s. Separate the phases by centrifugation, aspirate the aqueous (top) layer, add 200 μl methanolic hydrochloric acid (10 mmol HCl in methanol) to the remaining solution, mix gently, and evaporate the organic solvent under nitrogen in a water bath at 40° C. Wash the sides of the tube with 200 μl of methanolic hydrochloric acid and evaporate the solution. Reconstitute the residue in 100 μl of 30% methanol and inject 90 μl of it into the HPLC column.
(b) HPLC analysis: The chromatographic separation was performed with a column (Supelco 5-8347 LC-8-DB, 150×4.6 mm, 5 μm). Sample eluted with mobile phase of 0.34 M citric acid buffer: acetonitrile, 800: 45 (v/v) containing 0.34 M KH₂PO₄, 1-Heptane sulphonate (671 mg), and triethylamine (5 ml) with flow rate of 1.3 ml/min, and monitored by a UV detector at λ=260 nm.

Following determination of the concentration of nicotine and cotinine in the plasma by the HPLC method described above, the concentrations at 20, 30 and 60 minutes in the absence and presence of the pre-administration of the inhibitor are compared after subtracting any base line nicotine that is present. The average of these values or the Area Under the Concentration Curve can also be used. The degree of inhibition at each point (or average or area under the concentration curve) is expressed as percent inhibition=(concentration in the presence of the inhibitor−concentration nicotine in the absence of inhibitor)/(concentration in the absence of inhibitor)×100. The method is adaptable to accommodate inhibitors with varying kinetic properties in that longer of shorter sampling periods can be used. For example, some inhibitors may have a very long half-life and it might be desirable to obtain data over a longer period of time. The shorter screen test will nevertheless be sufficient for demonstrating the clinical inhibition and the therapeutic potential of the inhibitor. In some circumstance the ratio of nicotine/cotinine in plasma at these time points might be useful but for the purposes of screen for therapeutic efficacy we do not believe that cotinine concentrations are pertinent. The method described here can applied to non-smokers if the nicotine doses are decreased to 20 μg/kg s.c.

### Results:

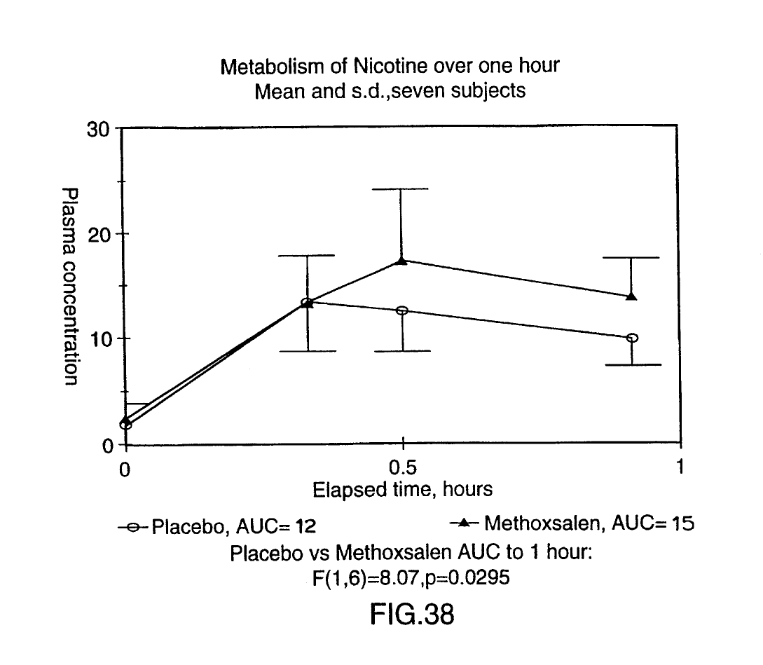

FIG. 38 is a graph showing the metabolism of nicotine over one hour in seven subjects. More specifically, the graph shows the changes in the plasma nicotine concentration level over time in the presence of methoxsalen, a CYP2A6 inhibitor, versus a placebo.

### (2) Nicotine Test of Inhibited Metabolism Stable Labelled Nicotine and Gas Chromatography Mass Spectrometry Methods

Under normal circumstances the above test will be widely usable and most appropriate. However when specialized analytical equipment is available another approach is also possible. The technique is described previously in this application. Simply, nicotine-d2 in a tracer dose is administered intravenously in the absence and presence of the inhibitor and plasma concentrations of nicotine and cotinine are measured by GC-MS in the plasma at time points similar to indicated in the previous section and the data treated in similar fashion. This method has advantages for research studies and for where the testing of inhibition must be done when the patients continue to smoke when inhibition is being tested.

### EXAMPLE 15

### Coumarin Phenotyping Test and CYP2A6 Genotyping Test 25

### A. Coumarin Test

Coumarin is a selective and specific substrate for human CYP2A6 and can be used to: (1) identify individuals who are potential therapeutic exclusions for use of CYP2A6 inhibitors; (2) for dosage refinement based on the initial level of activity of CYP2A6; and (3) for risk factor assessment in identifying individuals who will not benefit from the treatment or who may be at risk to toxicity from agents which are inhibitors and substrates themselves of CYP2A6. The Coumarin Test exists in two forms:

### (1) Coumarin Test When Only Urine is Available

Coumarin 5 mg formulated in a capsule or other dose form is administered orally to fasted individuals after voiding of residual bladder urine. Urine is collected for the first 2 hours and for the subsequent 6 hours. The amount of urinary excretion of the coumarin metabolite 7 hydroxy-coumarin (free and conjugated) is determined by determining the concentration of these metabolites on the urine using an HPLC assay as described in an earlier example. The relative activity of CYP2A6 is reflected in the total amounts of 7 hydroxy-coumarin excreted in the sampling periods separately and combined and the activity can be expressed as the ratio of the percent coumarin excretion (amount excreted in the first 2 hours/amount excreted in 8 hours)×100. This percent excretion ranges from values less then 20% in individuals without CYP2A6 activity to >80% in individuals with high activity. This test can be equally effectively and reliably be applied to smokers and non-smokers and may be used at any time of day with out apparent effect of the smoking condition or time of day on the results. The test demonstrates high within subject reproducibility with a linear r of >0.9. See FIG. 37 for results of a study in which smokers and nonsmokers were given coumarin in the morning and afternoon on each of 2 separate days. High within subject reproducibility and reliability is demonstrated.

(2) Coumarin Test when Plasma Samples can be Taken

In some clinical situations blood samples can be easily taken or are necessary as part of other clinical tests. In this situation, a plasma-based test of CYP2A6 activity has been developed and applied to individuals of known genotype. Individuals ingest coumarin 5.0 mg orally and 45 minutes later a blood sample is drawn in a heparinized (or other anticoagulant containing tube). The sample is spun and the plasma separated. The plasma is analyzed by HPLC to quantitate 7 hydroxycoumarin (total after deconjugation with beta glucuronidase incubation). High analytical sensitivity is required in order to use 5.0 mg of coumarin. When such sensitivity is not available, the dose of coumarin may be increased up to 50 mg.

### HPLC Analysis of 7-Hydroxycoumarin in Urine and Plasma:

### (1) Sample Preparation:

Urine or plasma samples (0.5 ml) are hydrolyzed with 0.2 ml of β-glucuronidase acetate buffer solution (15 mg/ml acetate buffer, 0.2 M, pH 5.0) at 37° C. for 30 min. Extraction is followed with 2 ml ether by vortex for 5 min and centrifuged at 3000 rpm for 10 min. Ether extract (1.2 ml) is transferred to another clean tube and dried down under nitrogen gas. The residue is reconstituted in the HPLC mobile phase (see below), and injected onto HPLC.

### (2) HPLC Analysis:

The HPLC system consists of Hewlett Packard 1050 HPLC system (pump, autosampler and UV detector) and HP3396II integrator. The chromatographic separation was performed with an HP Spherisorb-ODS2 column (125×4 mm I.D., 5 μm). Samples were eluted with a mobile phase of acetonitrile:water:acetic acid of 150:850:2 (v/v/v) at a flow rate of 1.0 ml/min, and monitored by a UV detector at a wavelength of 324 nm for 7-hydroxycoumarin and 280 nm for coumarin. Samples are quantitatively determined by an external standard method.

The CYP2A6 activity is expressed as the concentration of 7 hydroxy-coumarin in the plasma at various points in time (e.g. 20, 30, 45 and 75 minutes) or as the ratio of coumarin/7 hydroxy-coumarin in the plasma at that time.

The preferred mode of use is a simple plasma sample at 20 or 30 minutes after the oral administration of coumarin in which both coumarin and 7-hydroxycoumarin are quantified and in which the coumarin to 7-hydroxycoumarin ration is used as the index of CYP2A6 activity.

### Results:

Blank urine or plasma samples showed no interfering peak for 7-hydroxycoumarin or coumarin. Sensitivity of this method is 1 ng/ml urine or plasma. Intraday and inter-day variations are less than 10%. This analysis is linear from 1 ng to 4000 ng/ml.

FIG. 39 is a graph showing a time course of total 7-hydroxycoumarin concentration detected in the plasma of subjects given coumarin. FIG. 39 illustrates various time courses based on corresponding genotypes for CYP2A6.

### B. CYP2A6 Genotyping Test

As for the CYP2A6 genotyping test, mutant alleles which decrease CYP2A6 activity in an individual can be screened in a DNA sample using the materials and screening method described in Example 7.

Having illustrated and described the principles of the invention in a preferred embodiment, it should be appreciated to those skilled in the art that the invention can be modified in arrangement and detail without departure from such principles. We claim all modifications coming within the scope of the following claims.

All publications, patents and patent applications referred to herein are incorporated by reference in their entirety to the same extent as if each individual publication, patent or patent application was specifically and individually indicated to be incorporated by reference in its entirety.

Below full citations are set out for the references referred to in the specification.

1. A method of inhibiting the metabolism of nicotine to cotinine comprising administering to an individual an effective amount of at least one substance which selectively inhibits CYP2A6, wherein said individual has a condition selected from dependent tobacco use and non-dependent tobacco use.

2. The method of claim 1, wherein the individual maintains elevated plasma concentrations of nicotine compared to an individual who has not been administered a CYP2A6 inhibitor.

3. The method of claim 1, wherein liver enzyme function is inhibited by greater than 80% following administration of the CYP2A6 inhibitor.

4. The method of claim 1, wherein the condition is dependent tobacco use.

5. The method of claim 1, comprising optionally administering to an individual a mixture comprising two or more of said substances which selectively inhibits CYP2A6.

6. The method of claim 1, wherein the substance which selectively inhibits CYP2A6 is selected from antibodies specific for P4502A6, coumarin, 7-methoxycoumarin, 7-methylcoumarin, 7-ethoxycoumarin, furanocoumarin, methoxsalen, imperatorin, psoralen, α-naphthoflavone, isopimpinellin, p-naphthoflavone, bergapten, sphondin, coumatetralyl, (+)-cis-3,5-dimethyl-2-(3-pyridyl)-thiazolidim-4-one, naringenin, diethyldithiocarbamate, nitropyrene, menadione, imidazole antimycotics, hexamethylphosphoramide, 4-methylnitrosamine-3-pyridyl-1-butanol, aflatoxin B, indole, dihydrocoumarin, chomone, 3-isochromanone, 4,4′-methylene bis[2-chloroaniline], 6-aminochrysene, dicumarol, 4-chromanone, 4-chromanol, 1-naphthol, 1,3-indandione, 1-indanone, warfarin, sphondin, amgelicin, pimpinellin, a compound having the structure: 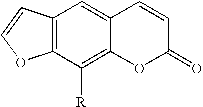
wherein R is —OCH₂CH₃, —OCH₂CH₂CH₃, —CH₂CH₂CH₃, —CH₂CH₂CH₂CH₃, —OH, —NH₂, —NO₂ or —C₆H₅;
a compound having the structure: 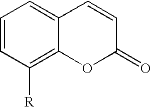
wherein R is —OCH₃, —OCH₂CH₃, —OCH₂CH₂CH₃, —OCH₂CH₂CH₂CH₃, —CH₃, —CH₂CH₃, —CH₂CH₂CH₃, CH₂CH₂CH₂CH₃, —OH, —NH₂, —NO₂ or —C₆H₅;
a compound having the structure: 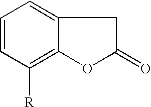
wherein R is —H, —OCH₃, —OCH₂CH₃, —OCH₂CH₂CH₃, —OCH₂CH₂CH₂CH₃, —CH₃, —CH₂CH₃, CH₂CH₂CH₃, CH₂CH₂CH₂CH₃, —OH, —NH₂, —NO₂ or —C₆H₅;
a compound having the structure: 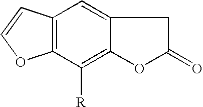
wherein R is —OCH₃, —OCH₂CH₃, —OCH₂CH₂CH₃, —OCH₂CH₂CH₂CH₃, —CH₃, —CH₂CH₃, CH₂CH₂CH₃, CH₂CH₂CH₂CH₃, —OH, —NH₂, —NO₂ or —C₆H₅;
a compound having the structure: 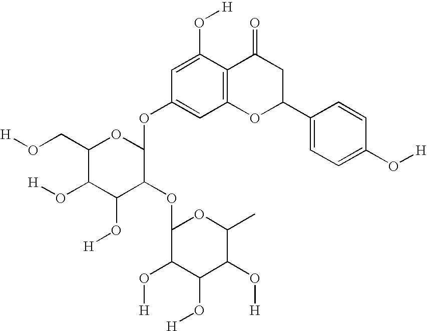
or a compound having the structure:

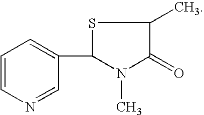

7. The method of claim 1, wherein the substance that selectively inhibits CYP2A6 is selected from coumarin, furanocoumarin, methoxsalen, imperatorin, psoralen, α-naphthoflavone, isopimpinellin, β-naphthoflavone, bergapten, sphondin, coumatetralyl (+)-cis-3,5-dimethyl-2-(3-pyridyl)-thiazolidim-4-one, naringenin, diethyldithiocarbamate, nitropyrene, menadione, imidazole antimycotics, pilocarpine, hexamethylphosphoramide, 4-methylnitrosamine-3-pyridyl-1-butanol, aflatoxin B, and mixtures thereof.

8. The method according to claim 7, wherein the imidazole antimycotic is selected from miconazole and clotrimazole

9. The method of claim 1, wherein said substance is formulated for slow release.
